(19) United States
(12) Patent Application Publication
(10) Pub. No.: US 2025/0320181 A1
(43) Pub. Date: Oct. 16, 2025

(54) SMALL MOLECULE INHIBITORS OF BACTERIAL TOXINS

(71) Applicant: Artizan Biosciences, Inc., New Haven, CT (US)

(72) Inventors: William HOEKSTRA, New Haven, CT (US); Hyunji RYU, New Haven, CT (US); Priyanka CHINTHA, New Haven, CT (US); Sammy R. SHAVER, New Haven, CT (US)

(21) Appl. No.: 18/694,627

(22) PCT Filed: Sep. 22, 2022
(86) PCT No.: PCT/US2022/076836

§ 371 Date: Mar. 22, 2024

Related U.S. Application Data
(60) Provisional application No. 63/248,094, filed on Sep. 24, 2021.

(51) Int. Cl. C07C 333/04; A61K 31/167; A61K 31/265; A61K 31/325; A61K 31/415; A61P 1/00; A61P 31/04; C07C 323/41; C07C 327/30; C07C 327/32; C07C 333/08; C07D 231/40

(52) U.S. Cl. CPC C07C 333/04; A61K 31/167; A61K 31/265; A61K 31/325; A61K 31/415; A61P 1/00; A61P 31/04; C07C 323/41; C07C 327/30; C07C 327/32; C07C 333/08; C07D 231/40

(57) ABSTRACT

Described herein are compounds and compositions for use in treatment or prevention of an inflammatory bowel disease, gastrointestinal cancer, or a systemic bacterial infection in a subject in need thereof. They subject may be colonized by one or more pathogenic bacterial strains such as B. fragilis, E. faecalis, or C. perfringens. In certain aspects, the disclosure provides a method of diminishing the pathogenic effects of these bacterial stmins by administering a compound that binds to and/or inhibits one or more toxins produced thereby.

### CROSS-REFERENCE TO RELATED APPLICATIONS

This application is a U.S. National Stage Application filed under 35 U.S.C. § 371 of International Application No. PCT/US2022/076836, filed Sep. 22, 2022, which claims the benefit of priority to U.S. Provisional Application No. 63/248,094, filed Sep. 24, 2021, which are hereby incorporated by reference in their entirety.

### FIELD OF THE DISCLOSURE

The present disclosure relates to compounds, compositions and methods for treating gastrointestinal diseases such as inflammatory bowel disease and gastrointestinal cancer. The present disclosure also relates to small molecule compounds, and compositions comprising the same, which bind to and/or inhibit toxins produced by various pathogenic bacterial strains.

### REFERENCE TO AN ELECTRONIC SEQUENCE LISTING

This application is being filed electronically via Patent Center and includes an electronically submitted sequence listing in .txt format. The contents of the electronic sequence listing (ARTI_008_02US_SubSeqList_ST26.xml; Size: 19,781 bytes; and Date of Creation: Apr. 7, 2025) are herein incorporated by reference in their entirety.

### BACKGROUND

Inflammatory bowel disease (IBD) is a group of inflammatory diseases of the colon and small intestine, including Crohn's disease and colitis. The most common forms of IBD are Crohn's disease and ulcerative colitis. Ulcerative colitis affects the large intestine (colon) and rectum and involves the inner lining (e.g., the mucosal and sub-mucosal layer) of the intestinal wall. Crohn's disease may affect any section of the gastrointestinal tract (e.g., mouth, esophagus, stomach, small intestine, large intestine, rectum, anus, etc.) and may involve all layers of the intestinal wall. The clinical symptoms of IBD include rectal and/or intestinal bleeding, abdominal pain and cramping, diarrhea, and weight loss. In addition, IBD is a risk factor for colon cancer, and this risk for colon cancer increases significantly after eight to ten years of IBD.

Although the etiology of IBD is unclear, experiments in animal models and humans have suggested that commensal bacteria play an important role in the pathogenesis of IBD. However, the exact nature of host-microbe interactions that contribute to IBD development is still unknown. Bacteria may contribute to IBD, for example, as causative agents, or may simply contribute to the perpetuation of the disease. Understanding bacterial functions in IBD can identify potential therapeutic approaches.

There is no cure for IBD, and currently available treatments do not work for all patients. Accordingly, there is a need in the art for improved compositions and methods for treating IBD.

### SUMMARY

The present disclosure is directed to compounds and compositions thereof that inhibit the activity of one or more pathogenic bacterial toxins, such as B. fragilis toxin (BFT), collagenase A (ColA) and gelatinase E (GelE). The disclosed compounds and compositions are useful in treating various diseases and disorders including inflammatory bowel disease, gastrointestinal cancer, and systemic bacterial infections in subjects in need thereof.

In some embodiments, the present disclosure provides a compound of Formula I:

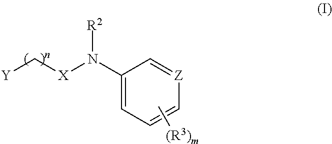

or a stereoisomer or a pharmaceutically acceptable salt thereof, wherein:
X is —C(O)— or —S(O)₂—;
Y is —SC(O)R¹ or —SH;
Z is CH or N;
R¹ is alkyl, haloalkyl, or —NR⁴R⁵;
R² is H, alkyl, or cycloalkyl;
R³ is —OH, alkoxy, —C(O)R⁶, or —C(O)N(H)-alkylene-C(O)N⁷R⁸, wherein the alkylene is optionally substituted;
R⁴, R⁵, R⁷, and R⁸ are each independently H, alkyl, cycloalkyl, —CH₂cycloalkyl, aryl, or —CH₂aryl;
R⁶ is alkyl, alkoxy, or —N(H)alkyl;
m is 0, 1, 2, or 3; and
n is 1 or 2.

In some embodiments of Formula I, X is —C(O)—.

In some embodiments of Formula I, R¹ is C₁₋₅alkyl or —NR⁴R⁵. In some embodiments, the C₁₋₅alkyl is methyl, ethyl, or isopropyl. In some embodiments, the C₁₋₅alkyl is methyl. In some embodiments, R⁴ and R⁵ are each H. In some embodiments, R⁴ is H and R⁵ is methyl.

In some embodiments of Formula I, R² is H or alkyl. In some embodiments, R² is H.

In some embodiments of Formula I, R³ is alkoxy, —C(O)R⁶, or —C(O)N(H)-alkylene-C(O)N⁷R⁸. In some embodiments, the alkylene is a methylene, optionally substituted with fluoro, alkyl, or —CH₂aryl. In some embodiments, R³ is alkoxy or —C(O)R⁶. In some embodiments, the alkoxy is —OMe. In some embodiments, R⁶ is Me or —OMe. In some embodiments, R⁷ and R⁸ are each independently H, Me, or —CH₂Ph. In some embodiments, R⁷ is H and R⁸ is Me.

In some embodiments of Formula I, Z is CH.

In some embodiments of Formula I, m is 1 or 2. In some embodiments, m is 1.

In some embodiments of Formula I, n is 1. In some embodiments, n is 2.

In some embodiments, the present disclosure provides a compound of Formula II or Formula IIA:

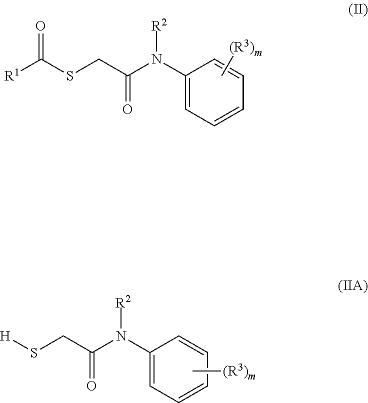

or a stereoisomer or a pharmaceutically acceptable salt thereof, wherein:
R¹ is alkyl, haloalkyl, or —NR⁴R⁵;
R² is H, alkyl, or cycloalkyl;
R³ is —OH, alkoxy, —C(O)R⁶, or —C(O)N(H)-alkylene-C(O)N⁷R⁸, wherein the alkylene is optionally substituted;
R⁴, R⁵, R⁷, and R⁸ are each independently H, alkyl, cycloalkyl, —CH₂cycloalkyl, aryl, or —CH₂aryl;
R⁶ is alkyl, alkoxy, or —N(H)alkyl; and
m is 0, 1, 2, or 3.

In some embodiments of Formula II, R¹ is C₁₋₅alkyl or —NR⁴R⁵. In some embodiments, the C₁₋₅alkyl is methyl, ethyl, or isopropyl. In some embodiments, the C₁₋₅alkyl is methyl. In some embodiments, R⁴ and R⁵ are each H. In some embodiments, R⁴ is H and R⁵ is methyl.

In some embodiments of Formula II, R² is H or alkyl. In some embodiments, R² is H.

In some embodiments of Formula II, R³ is alkoxy, —C(O)R⁶, or —C(O)N(H)-alkylene-C(O)N⁷R⁸. In some embodiments, the alkylene is a methylene, optionally substituted with fluoro, alkyl, or —CH₂aryl. In some embodiments, R³ is alkoxy or —C(O)R⁶. In some embodiments, the alkoxy is —OMe. In some embodiments, R⁶ is Me or —OMe. In some embodiments, R⁷ and R⁸ are each independently H, Me, or —CH₂Ph. In some embodiments, R⁷ is H and R⁸ is Me.

In some embodiments of Formula II, m is 1 or 2. In some embodiments, m is 1.

In some embodiments, the present disclosure provides a compound of Formula V:

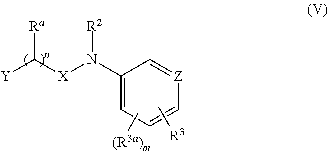

or a stereoisomer or a pharmaceutically acceptable salt thereof, wherein:
X is —C(O)— or —S(O)₂—;
Y is —SC(O)R¹ or —SH;
Z is CH or N;
R^{a} is each independently H, alkyl, haloalkyl, aryl, alkylene-cycloalkyl, alkylene-heterocycloalkyl, heteroaryl, or alkylene-aryl, alkylene-heteroaryl;
R¹ is alkyl, haloalkyl, or —NR⁴R⁵;
R² is H, alkyl, or cycloalkyl;
R³ is —OH, alkoxy, —O-alkylene-NR⁷R⁸, —C(O)R⁶, or —C(O)N(H)-alkylene-C(O)NR⁷R⁸, wherein the alkylene is optionally substituted;
R^{3a} is alkyl, halogen, alkoxy, haloalkyl or haloalkoxy;
R⁴, R⁵, R⁷, and R⁸ are each independently H, alkyl, cycloalkyl, —CH₂cycloalkyl, aryl, or —CH₂aryl;
R⁶ is alkyl, haloalkyl, alkoxy, or —N(H)alkyl;
m is 0, 1, 2, or 3; and
n is 1 or 2,
wherein when n is 1, R^{a} is not H.

In some embodiments of Formula V, R¹ is C₁₋₅alkyl or —NR⁴R⁵. In some embodiments, the C₁₋₅alkyl is methyl, ethyl, or isopropyl. In some embodiments, the C₁₋₅alkyl is methyl. In some embodiments, R⁴ and R⁵ are each H. In some embodiments, R⁴ is H and R⁵ is methyl. In some embodiments, —NR⁴R⁵ is —NH₂, —NH(Me), —NH(i-Pr), —NH(t-Bu), —N(CH₃)₂, or H,

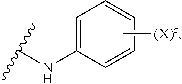

wherein X is Cl or F and z is 0, 1, 2, or 3. In some embodiments, z is 1 or 2. In some embodiments, z is 1. In some embodiments, z is 2.

In some embodiments of Formula V, R² is H or alkyl. In some embodiments, R² is H.

In some embodiments of Formula V, R³ is alkoxy, haloalkoxy, —C(O)R⁶, or —C(O)N(H)-alkylene-C(O)N⁷R⁸. In some embodiments of Formula V, R³ is alkoxy, —C(O)R⁶, or —C(O)N(H)-alkylene-C(O)N⁷R⁸. In some embodiments of Formula V, R³ is alkoxy, —C(O)R⁶, or —O-alkylene-NR⁷R⁸. In some embodiments, the alkylene is a C₁₋₃alkylene optionally substituted with oxo, alkyl, alkylene-cycloalkyl, alkylene-heterocycloalkyl, alkylene-aryl, or alkylene-heteroaryl. In some embodiments, the —O-alkylene-NR⁷R⁸ is

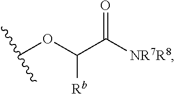

wherein R^{b} is H, alkyl, alkylene-cycloalkyl, alkylene-heterocycloalkyl, alkylene-aryl, or alkylene-heteroaryl. In some embodiments, the —C(O)N(H)-alkylene-C(O)NR⁷R⁸ is

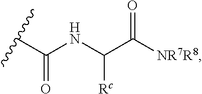

wherein R^{c} is H, alkyl, alkylene-cycloalkyl, alkylene-heterocycloalkyl, alkylene-aryl, or alkylene-heteroaryl. In some embodiments, R⁷ and R⁸ are each independently H, Me, or —CH₂Ph. In some embodiments, R⁷ is H and R⁸ is Me. In some embodiments, R³ is alkoxy or —C(O)R⁶. In some embodiments, the alkoxy is —OMe. In some embodiments, R⁶ is Me or —OMe.

In some embodiments of Formula V, m is 1 or 2. In some embodiments, m is 1.

In some embodiments of Formula V, n is 1.

In some embodiments of Formula V, when n is 2, one R^{a} is H.

In some embodiments, the present disclosure provides a compound of Formula VA:

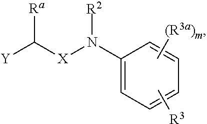

or a stereoisomer or a pharmaceutically acceptable salt thereof, wherein:
X is —C(O)— or —S(O)₂—;
Y is —SC(O)R¹ or —SH;
R^{a} is alkyl, haloalkyl, aryl, alkylene-cycloalkyl, alkylene-heterocycloalkyl, heteroaryl, alkylene-aryl, or alkylene-heteroaryl;
R¹ is alkyl, haloalkyl, or —NR⁴R⁵;
R² is H, alkyl, or cycloalkyl;
R³ is —OH, alkoxy, —O-alkylene-NR⁷R⁸, —C(O)R⁶, or —C(O)N(H)-alkylene-C(O)NR⁷R⁸, wherein the alkylene is optionally substituted;
R³ is alkyl, halogen, alkoxy, haloalkyl or haloalkoxy;
R⁴, R⁵, R⁷, and R⁸ are each independently H, alkyl, cycloalkyl, —CH₂cycloalkyl, aryl, or —CH₂aryl;
R⁶ is alkyl, haloalkyl, alkoxy, or —N(H)alkyl; and
m is 0, 1, 2, or 3.

In some embodiments, the present disclosure provides a compound of Formula VB:

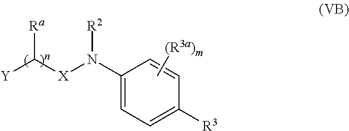

or a stereoisomer or a pharmaceutically acceptable salt thereof, wherein:
X is —C(O)— or —S(O)₂—;
Y is —SC(O)R¹, —SH;
R^{a} is H, alkyl, haloalkyl, aryl, alkylene-cycloalkyl, alkylene-heterocycloalkyl, heteroaryl, alkylene-aryl, or alkylene-heteroaryl;
R¹ is alkyl, haloalkyl, or —NR⁴R⁵;
R² is H, alkyl, or cycloalkyl;
R³ is —OH, alkoxy, —O-alkylene-NR⁷R⁸, —C(O)R⁶, or —C(O)N(H)-alkylene-C(O)NR⁷R⁸, wherein the alkylene is optionally substituted;
R³ is alkyl, halogen, alkoxy, haloalkyl or haloalkoxy;
R⁴, R⁵, R⁷, and R⁸ are each independently H, alkyl, cycloalkyl, —CH₂cycloalkyl, aryl, or —CH₂aryl;
R⁶ is alkyl, haloalkyl, alkoxy, or —N(H)alkyl;
m is 0, 1, 2, or 3; and
n is 1 or 2,
wherein when n is 1, R^{a} is not H.

In some embodiments, the compound of Formula VB has the structure:

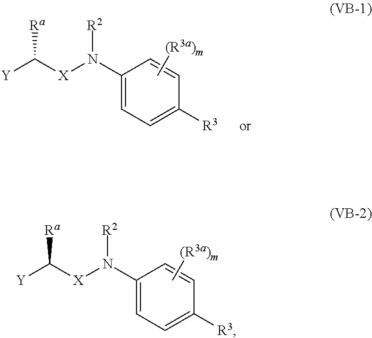

or a pharmaceutically acceptable salt thereof.

In some embodiments, the present disclosure provides a pharmaceutical composition comprising a compound of Formula I, Formula II, Formula III, Formula IV, Formula V, Formula VA, Formula VB, Formula VC, or Formula VD and a pharmaceutically acceptable carrier or excipient.

Provided herein are methods of treating inflammatory bowel disease in a subject in need thereof, the method comprising, administering to the subject a therapeutically effective amount of a compound of the present disclosure (e.g., a compound of Formula I, Formula II, Formula III, Formula IV, Formula V, Formula VA, Formula VB, Formula VC, or Formula VD). In some embodiments, the inflammatory bowel disease is Crohn's disease or ulcerative colitis.

Also provided herein are methods of treating gastrointestinal cancer in a subject in need thereof, the methods comprising, administering to the subject a therapeutically effective amount of a compound of the present disclosure (e.g., a compound of Formula I, Formula II, Formula IIA, Formula III, Formula IV, Formula V, Formula VA, Formula VB, Formula VC, or Formula VD). In some embodiments, the GI cancer is esophageal cancer, gallbladder cancer, liver cancer, pancreatic cancer, stomach cancer, cancer of the small intestine, colorectal cancer, or anal cancer.

Also provided herein are methods of treating a systemic bacterial infection in a subject in need thereof, the methods comprising, administering to the subject a therapeutically effective amount of a compound of the present disclosure (e.g., a compound of Formula I, Formula II, Formula III, Formula IV, Formula V, Formula VA, Formula VB, Formula VC, or Formula VD). In some embodiments, the systemic bacterial infection in endocarditis or a urinary tract infection.

In some embodiments, the subject is colonized by one or more pathogenic bacterial strains. In some embodiments, the pathogenic bacterial strain is B. fragilis, E. faecalis, and/or C. perfringens. In some embodiments, the pathogenic bacterial strain is a strain of B. fragilis expressing the BFT toxin. In some embodiments, the pathogenic bacterial strain is a strain of E. faecalis expressing the gelatinase GelE. In some embodiments, the pathologic bacterial strain is a strain of C. perfringens expressing the collagenase ColA.

In some embodiments, administering a compound of the present disclosure reduces and/or eliminates the activity of at least one of BFT, ColA and/or GelE in the subject. In some embodiments, administering the compound reduces and/or eliminates the activity of BFT in the subject. In some embodiments, administering the compound results in a reduction in the number of B. fragilis, E. faecalis, and/or C. perfringens in the subject.

In some embodiments, a compound of the present disclosure binds to and/or inhibits one or more of B. fragilis toxin (BFT), collagenase A (ColA), and gelatinase E (GelE). In some embodiments, the compound binds to BFT, ColA, and/or GelE with an inhibition constant (Kᵢ) in the range of about 10⁻⁵ M to about 10⁻¹³ M. In some embodiments, the BFT comprises the amino acid sequence of any one of SEQ ID NO: 2-4. In some embodiments, the BFT comprises an amino acid sequence that is at least 90%, at least 95%, or at least 98% identical to any one of SEQ ID NO: 2-4. In some embodiments, the GelE comprises the amino acid sequence of SEQ ID NO: 6. In some embodiments, the GelE comprises an amino acid sequence that is at least 90%, at least 95%, or at least 98% identical to SEQ ID NO: 6. In some embodiments, the ColA comprises the amino acid sequence of SEQ ID NO: 8. In some embodiments, the ColA comprises an amino acid sequence that is at least 90%, at least 95%, or at least 98% identical to SEQ ID NO: 8.

In some embodiments, the compound is administered intravenously to the subject. In some embodiments, the compound is administered orally to the subject. In some embodiments, the compound is administered in a tablet or a capsule, wherein the tablet or capsule optionally comprises a pharmaceutically acceptable carrier or excipient. In some embodiments, the compound is administered as a liquid formulation, wherein the liquid formulation optionally comprises a pharmaceutically acceptable carrier or excipient.

In some embodiments, the compound is administered once per day, once per week, or multiple times per day or week. In some embodiments, the dose of the compound administered to the subject is from about 0.001 to about 1000 mg/kg of body weight per day.

These and other aspects are described in greater detail below.

### BRIEF DESCRIPTION OF THE DRAWINGS

FIG. 1 shows a crystal structure of BFT, a zinc-dependent metalloprotease. The inset shows the zinc-binding domain. BFT is produced by the cell as an inactive protease comprising an inhibitory pro-domain which inserts itself into the active site of the enzyme to inhibit toxin activity. The pro-domain is cleaved by a protease (e.g., fragipain or other host proteases such as trypsin) to produce an active toxin. FIG. 1 is adapted from Goulas, et al., PNAS (2010).

FIG. 2 shows a schematic of the cell-based BFT toxicity assay for screening test compounds. Recombinant BFT is pre-incubated with one or more test compounds. The BFT-inhibitor mixture is applied to a cell monolayer. After 18 hours of incubation at 37° C., cellular supernatants are collected. The activity of BFT may be quantified by measuring E-cadherin or IL-8 levels in the supernatant, for example using a standard ELISA.

FIG. 3 is a schematic of the ETBF-mediated disease model for screening test compounds in vivo. Germ-free (GF) mice were mono-colonized with ETBF on day 0. On days 1, 2, and 3 following colonization, mice were orally administered 50 mg/kg of the test compound two times per day (BID). Markers of inflammation were analyzed on day 4.

FIG. 4 shows percent inhibition of ColH inhibition (a surrogate for ColA inhibition) following treatment with 512 pM to 200 μM of compound 1.

FIG. 5 is a Clostridium perfringens dose-response curve for collagenase inhibition by Compound 24.

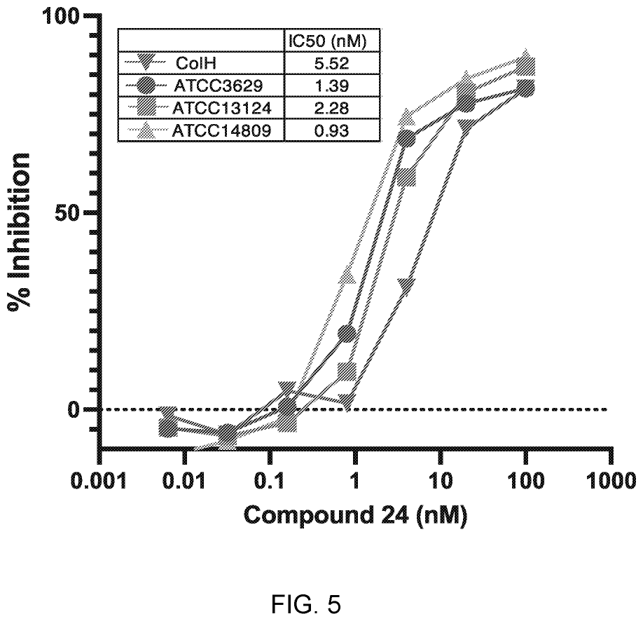

### DETAILED DESCRIPTION

Provided herein are compounds, e.g., small molecule inhibitors of BFT, GelE, and/or ColA, that are useful in treating a disease or disorder in subject in need thereof. In some embodiments, the disease or disorder is an inflammatory bowel disease, gastrointestinal cancer, or a systemic bacterial infection and the subject is colonized by one or more pathogenic bacterial strains, e.g., B. fragilis, E. faecalis, and/or C. perfringens.

As described herein, B. fragilis, E. faecalis and C. perfringens have been identified as causative agents that contribute to the development and progression of inflammatory bowel diseases (IBD) such as ulcerative colitis and Crohn's disease, and may therefore be targeted in the prevention and/or treatment thereof. Strains of each of these three bacterial species produce toxins (BFT from B. fragilis, GelE from E. faecalis, and ColA from C. perfringens) that are believed to contribute to the pathogenesis of IBD, and are therefore therapeutic targets. Compounds of the present disclosure (e.g., a compound of Formula I, Formula II, Formula IIA, Formula III, or Formula IV) bind and/or inhibit the activity of these toxins in vitro and/or in vivo, and may therefore be used to treat or prevent IBD and other gastrointestinal diseases in subjects in need thereof.

### Definitions

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “treating” as used herein with regard to a patient, refers to improving at least one symptom of the patient's disorder. Treating can be improving, or at least partially ameliorating a disorder. For purposes of the present disclosure, treating includes, but is not limited to improving, or at least partially ameliorating the effects of IBD, gastrointestinal cancer, a systemic bacterial infection and related conditions.

The terms “administer,” “administering” or “administration” as used herein refer to either directly administering a compound or pharmaceutically acceptable salt or ester of the compound or a composition comprising the compound or pharmaceutically acceptable salt or ester of the compound to a patient.

A disease or disorder is “alleviated,” “ameliorated” or “improved” if the severity of a sign or symptom of the disease or disorder, the frequency with which such a sign or symptom is experienced by a patient, or both, is reduced.

An “effective amount” or “therapeutically effective amount” of a compound is that amount of a compound which is sufficient to provide a beneficial effect to the subject to which the compound is administered.

The terms “patient,” “subject,” “individual,” and the like are used interchangeably herein, and refer to any animal, or cells thereof whether in vitro or in vivo, amenable to the methods described herein. In certain non-limiting embodiments, the patient, subject or individual is, by way of non-limiting examples, a human, a dog, a cat, a horse, or other domestic mammal.

As used herein, a “pharmaceutical composition” is meant to encompass a composition suitable for administration to a subject, such as a mammal, especially a human. In general a “pharmaceutical composition” is sterile, and is usually free of contaminants that are capable of eliciting an undesirable response within the subject (e.g., the compound(s) in the pharmaceutical composition is(are) pharmaceutical grade). Pharmaceutical compositions can be designed for administration to subjects or patients in need thereof via a number of different routes of administration including oral, buccal, rectal, parenteral, intraperitoneal, intradermal, intratracheal and the like.

The phrase “pharmaceutically acceptable excipient” means an excipient that is useful in preparing a pharmaceutical composition that is generally safe, non-toxic and neither biologically nor otherwise undesirable, and includes an excipient, that is acceptable for veterinary use as well as human pharmaceutical use. “A pharmaceutically acceptable excipient” as used in the specification and claims includes both one and more than one such excipient.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine. The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

The term “small molecule” generally refers to a compound having a molecular weight less than or equal to 700 daltons. In some embodiments, a “small molecule” has a molecular weight less than or equal to 600 daltons, 500 daltons, or 400 daltons, or 300 daltons. In some embodiments, a “small molecule” has a molecular weight less than or equal to about 400 daltons. In some embodiments, a “small molecule” has a molecular weight less than or equal to about 300 daltons. In the present disclosure, the term “small molecule” may be used interchangeably with “compound” or “compound of the present disclosure” or any other term that refers to a compound of the present disclosure without out altering meaning.

The term “amino acid” includes, but is not limited to, the group comprising of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The terms “peptide”, “polypeptide”, and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds.

The term “alkyl” as used herein refers to a branched or straight chain alkyl, wherein alkyl chain length is indicated by a range of numbers. In some embodiments, “straight chain alkyl” refers to an alkyl chain as defined above containing 1, 2, 3, 4, 5, or 6 carbons (i.e., C1-C6 alkyl). Examples of a straight chain alkyl group include, but are not limited to, methyl, ethyl, propyl, butyl, pentyl, and hexyl. In some embodiments, “branched alkyl” refers to an alkyl chain as defined above containing from 3, 4, 5, 6, 7, or 8 carbons (i.e., branched C3-C8 alkyl). Examples of a branched alkyl group include, but are not limited to, isopropyl, isobutyl, secondary-butyl, tertiary-butyl, isoamyl, and isopentyl. Unless stated otherwise specifically in the specification, an alkyl group can be optionally substituted.

The term “alkoxy” as used herein refers to —O-(alkyl), wherein “alkyl” is as defined above as a branched or straight chain alkyl. Unless stated otherwise specifically in the specification, an alkoxy group can be optionally substituted.

The term “alkylene” as used herein refers to a divalent alkyl moiety interposed between two other atoms. In exemplary embodiments, “alkylene” refers to an alkyl moiety as defined above containing 1, 2, or 3 carbons. Examples of an alkylene group include, but are not limited to —CH₂—, —CH₂CH₂—, and —CH₂CH₂CH₂— In exemplary embodiments, alkylene groups are branched. Unless stated otherwise specifically in the specification, an alkylene group can be optionally substituted.

The term “aryl” as used herein refers to a cyclic hydrocarbon, where the ring is characterized by delocalized π electrons (aromaticity) shared among the ring members, and wherein the number of ring atoms is indicated by a range of numbers. In exemplary embodiments, “aryl” refers to a cyclic hydrocarbon as described above containing 6, 7, 8, 9, or 10 ring atoms (i.e., C6-C10 aryl). Examples of an aryl group include, but are not limited to, benzene, naphthalene, tetralin, indene, and indane. Unless stated otherwise specifically in the specification, an aryl group can be optionally substituted.

The term “aralkyl” as used herein means an aryl group, as defined herein, appended to the parent molecular moiety through an alkyl group, as defined herein. Representative examples of aralkyl include, but are not limited to, benzyl, 2-phenylethyl, 3-phenylpropyl, and 2-naphth-2-ylethyl. Unless stated otherwise specifically in the specification, an aralkyl group can be optionally substituted.

The term “haloalkyl” means an alkyl group, as defined herein, wherein at least one hydrogen is replaced with a halogen, as defined herein. Representative examples of haloalkyl include, but are not limited to, chloromethyl, 2-fluoroethyl, trifluoromethyl, difluoromethyl, fluoromethyl, pentafluoroethyl, 2,2,2-trifluoroethyl, and 2-chloro-3-fluoropentyl. In some embodiments, the haloalkyl is a C₁₋₂ fluoralkyl having from 1-5 fluorides. Non-limiting examples include CF3, CF₂H, CFH₂, CH₂CF₃, and CF₂CF₃. Unless stated otherwise specifically in the specification, a haloalkyl group can be optionally substituted.

The term “halogen” as used herein refers to fluorine, chlorine, bromine, and iodine.

The term “heteroaryl” as used herein refers to a cyclic ring system, wherein at least one of the ring atoms is an O, N, or S, at least one ring is aromatic, and wherein the number of ring atoms can be indicated by a range of numbers (e.g., 5- to 12-membered heteroaryl, 5- to 7-membered heteroaryl, 5-membered heteroaryl, or 6-membered heteroaryl). Heteroaryl moieties as defined herein can be bound by a single bond to other moieties via one or more C or N atoms in the ring. For example, in some embodiments, a ring N atom from the heteroaryl is the bonding atom to —C(O) to form an amide, carbamate, or urea. In exemplary embodiments, “heteroaryl” refers to a cyclic hydrocarbon as described above containing 5 or 6 ring atoms. In some embodiments, the heteroaryl is a monocyclic heteroaryl. Examples of a monocyclic heteroaryl group include, but are not limited to, pyrrole, furan, thiene, oxazole, thiazole, isoxazole, isothiazole, imidazole, pyrazole, oxadiazole, thiadiazole, triazole, tetrazole, pyridine, pyrimidine, pyrazine, pyridazine, and triazine. In some embodiments, the heteroaryl is a bicyclic heteroaryl. Examples of a bicyclic heteroaryl group include, but are not limited to, quinoline, isoquinoline, quinazoline, cinnoline, phthalazine, quinazoline, quinoxaline, indolyl, benzoxazole, benzthiazole, and benzimidazole. Unless stated otherwise specifically in the specification, a heteroaryl group can be optionally substituted.

The term “heteroaralkyl” as used herein means a heteroaryl, as defined herein, appended to the parent molecular moiety through an alkyl group, as defined herein. Representative examples of heteroaralkyl include, but are not limited to, pyridin-3-ylmethyl and 2-(thien-2-yl)ethyl. Unless stated otherwise specifically in the specification, a heteroaralkyl group can be optionally substituted.

As used herein, “pyridyl” refers to a group derived from pyridine by removal of a hydrogen atom from a ring carbon atom. In some embodiments, the pyridyl is a 3-pyridyl, 4-pyridyl, or 5-pyridyl. Unless stated otherwise specifically in the specification, a pyridyl group can be optionally substituted.

The term “heterocyclyl” as used herein refers to a saturated or partially unsaturated cyclic ring system wherein at least one of the ring atoms is an O, N, or S and wherein the number of ring atoms can be indicated by a range of numbers (e.g., 4- to 12-membered heterocyclyl, 4- to 7-membered heterocyclyl, 5-membered heterocyclyl, or 6-membered heterocyclyl). Heterocyclyl moieties as defined herein can be bound by a single bond to other moieties via one or more C or N atoms in the ring. For example, in some embodiments, a ring N atom from the heterocyclyl is the bonding atom to —C(O) to form an amide, carbamate, or urea. In some embodiments, the heterocyclyl ring is a monocyclic or bicyclic heterocyclyl ring. In some embodiments, the heterocyclyl ring is a monocyclic heterocyclyl ring. Non-limiting examples of heterocyclyl rings include, but are not limited to, azetidinyl, pyrrolidinyl, piperidinyl, morpholinyl, thiomorpholinyl, thiolanyl, and tetrahydrofuranyl. Unless stated otherwise specifically in the specification, a heterocyclyl group can be optionally substituted.

The term “substituted” as used herein means any of the groups described herein (e.g., alkyl, alkenyl, alkynyl, alkoxy, aryl, aralkyl, carbocyclyl, cycloalkyl, cycloalkenyl, cycloalkynyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O) NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds of the Disclosure

Provided herein are compounds that are useful in treating various diseases and disorders, including diseases and disorders of the gastrointestinal tract. In some embodiments, the compounds of the present disclosure are capable of inhibiting one or more toxins produced by pathogenic bacterial strains. In some embodiments, the pathogenic bacterial strain is B. fragilis, E. faecalis, and/or C. perfringens.

B. fragilis and B. fragilis Toxin (BFT)

In some embodiments, the pathogenic bacterial strain is B. fragilis. B. fragilis is a gram-negative, rod-shaped bacterium, and may be identified by its 16S RNA sequence (see Table 1, below). For example, in some embodiments, a strain of B. fragilis has a sequence that is at least 90%, at least 95%, at least 96%, at least 97%, at least 98%, or at least 99% identical to the sequence of SEQ ID NO: 1. In some embodiments, a strain of B. fragilis has a 16S RNA sequence that is at least 97% identical to the sequence of SEQ ID NO: 1.

TABLE 1
Sequence encoding B. fragilis 16S RNA

| | | SEQ ID |
|---|---|---|
| Species | Sequence | NO: |
| B. fragilis | GCGCGATTACTAGCGAATCCAGCTTCACGAAGTCGGG | 1 |
| | TTGCAGACTTCGATCCGAACTGAGAGAGGATTTTGGGA | |
| | TTAGCATACGGTCACCCGCTAGCTGCCTTCTGTACCCC | |
| | CCATTGTAACACGTGTGTAGCCCCGGACGTAAGGGCC | |
| | GTGCTGATTTGACGTCATCCCCACCTTCCTCACATCTT | |
| | ACGACGGCAGTCTCTCCAGAGTCCTCAGCATGACCTG | |
| | TTAGTAACTGAAGATAAGGGTTGCGCTCGTTATGGCAC | |
| | TTAAGCCGACACCTCACGGCACGAGCTGACGACAACC | |
| | ATGCAGCACCTTCACAGCGGTGATTGCTCACTGACATG | |
| | TTTCCACATCATTCCACTGCAATTTAAGCCCGGGTAAG | |
| | GTTCCTCGCGTATCATCGAATTAAACCACATGTTCCTC | |
| | CGCTTGTGCGGGCCCCCGTCAATTCCTTTGAGTTTCAC | |
| | CGTTGCCGGCGTACTCCCCAGGTGGAATACTTAATGC | |
| | TTTCGCTTGGCCGCTTACTGTATATCGCAAACAGCGAG | |
| | TATTCATCGTTTACTGTGTGGACTACCAGGGTATCTAAT | |
| | CCTGTTTGATACCCACACTTTCGAGCATCAGTGTCAGT | |
| | TGCAGTCCAGTGAGCTGCCTTCGCAATCGGAGTTCTTC | |
| | GTGATATCTAAGCATTTCACCGCTACACCACGAATTCC | |
| | GCCCACCTCTACTGTACTCAAGACTGACAGTATCAACT | |
| | GCAATTTTACGGTTGAGCCGCARACTTTCACAACTGAC | |
| | TTACCAGTCCACCTACGCTCCCTTTAAACCCAATAAAT | |
| | CCGGATAACGCTCGGATCCTCCGTATTACCGCGGCTG | |
| | CTGGCACGGAGTTAGCCGATCCTTATTCATATAATACA | |
| | TACAAAACAGTATACATACTGCACTTTATTCTTATATAA | |
| | AAGAAGTTTACGACCCATAGAGCCTTCATCCTTCACGC | |
| | TACTTGGCTGGTTCAGGCTAGCGCCCATTGACCAATAT | |
| | TCCTCACTGCTGCCTCCCGTAGGAGTTTGGACCGTGT | |
| | CTCAGTTCCAATGTGGGGGACCTTCCTCTCAGAACCC | |
| | CTATCCATCGAAGGCTTGGTGAGCCGTTACCTCACCAA | |
| | CAACCTAATGGAACGCATCCCCATCCTTTACCGGAATC | |
| | CTTTAATAATGAAACCATGCGGAATCATTATGCTATCG | |
| | GGTATTAATCTTTCTTTCGAAAGGCTATCCCCGAGTAA | |
| | AGGGCAGGTTGGATACGTGTTACTCACCCGTGCGCCG | |
| | GTCGCCGGCAAAGAAAGCAAGCTTTCTT | |

B. fragilis (Bacteroides fragilis) is a common commensal anaerobe (about 0.5% of the human colonic flora) that shapes the host health, including the immune system. Some pathogenic strains of B. fragilis, including enterotoxigenic B. fragilis (ETBF) strains, harbor a gene encoding a pro-inflammatory enterotoxin called B. fragilis toxin (BFT) or fragilysin.

BFT, a ˜20 kDa zinc-dependent metalloprotease toxin, is secreted from ETBF strains. BFT reversibly stimulates chloride secretion and alters tight junctional function in polarized intestinal epithelial cells. Experimental studies originally suggested that the cellular target for BFT was E-cadherin, but more recent studies have suggested that BFT binds to a different, unidentified host receptor. BFT's enzymatic activity is required for ETBF's pathogenicity.

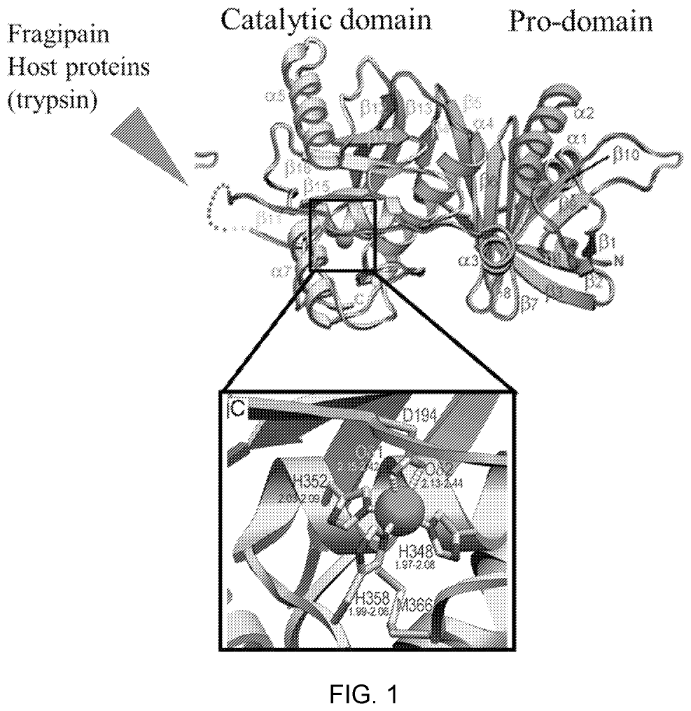

Enterotoxigenic strains of B. fragilis (i.e., ETBF strains) have genes encoding a pro-inflammatory enterotoxin called BFT (FIG. 1). These strains may be differentiated from non-toxigenic strains (i.e., NTBF strains) using several methods known to those of skill in the art, such as by using PCR to detect BFT genes in a B. fragilis sample. Exemplary ETBF strains include 86-5443-2-2, 2-078382-3, BOB25, 20656-2-1, 20793-3, 2078382-3, 20793-3, 20656-2-1, 86-5443-2-2. In some embodiments, an ETBF strain is isolated from a human fecal sample. In some embodiments, an ETBF strain is an engineered strain, such as a non-toxigenic B. fragilis strain engineered to express or overexpress BFT.

There are three known isotypes of BFT, encoded by distinct bft loci contained within a 6 kb chromosomal region found in ETBF strains termed the B. fragilis pathogenicity island (BfPAI). Various BFT isotypes are listed in Table 2, below. In some embodiments, an ETBF strain expresses at least one of BFT1, BFT2, and/or BFT3.

TABLE 2
B. fragilis toxin (BFT) isotypes

| Name | Sequence | SEQ ID No: |
|---|---|---|
| BFT1 | MFILNFNKMKNVKLLLMLGTAALLAACSNEADSLTTSIDAPVTAS | 2 |
| | IDLQSVSYTDLATQLNDVSDFGKMIILKDNGFNRQVHVSMDKRT | |
| | KIQLDNENVRLFNGRDKDSTSFILGDEFAVLRFYRNGESISYIAY | |
| | KEAQMMNEIAEFYAAPFKKTRAINEKEAFECIYDSRTRSAGKDI | |
| | VSVKINIDKAKKILNLPECDYINDYIKTPQVPHGITESQTRAVPSE | |
| | PKTVYVICLRENGSTIYPNEVSAQMQDAANSVYAVHGLKRYVN | |
| | FHFVLYTTEYSCPSGDAKEGLEGFTASLKSNPKAEGYDDQIYFL | |
| | IRWGTWDNKILGMSWFNSYNVNTASDFEASGMSTTQLMYPGV | |
| | MAHELGHILGAEHTDNSKDLMYATFTGYLSHLSEKNMDIIAKNL | |
| | GWEAADGD | |
| BFT2 | MKNVKLLLMLGTAALLAACSNEADSLTTSIDTPVTASIDLQSVSY | 3 |
| | TDLATQLNDVSDFGKMIILKDNGFNRQVHVSMDKRTKIQLDNE | |
| | NVRLFNGRDKDSTSFILGDEFAVLRFYRNGESISYIAYKEAQMM | |
| | NEIAEFYAAPFKKTRAINEKEAFECIYDSRTRSAGKDLVSVKINID | |
| | KAKKILNLPECDYINDYIKTPQVPHGITESQTRAVPSEPKTVYVIC | |
| | LRESGSTVYPNEVSAQMQDAANSVYAVHGLKRFVNLHFVLYTT | |
| | EYSCPSGNADEGLDGFTASLKANPKAEGYDDQIYFLIRWGTWD | |
| | NNILGISWLDSYNVNTASDFKASGMSTTQLMYPGVMAHELGHI | |
| | LGARHADDPKDLMYSKYTGYLFHLSEENMYRIAKNLGWEIADG | |
| | D | |
| BFT3 | MKNVKLLLMLGTAALLAACSNEADSLTTSIDAPVTASIDLQSVSY | 4 |
| | TDLATQLNDVSDFGKMIILKDNGFNRQVHVSMDKRTKIQLDNE | |
| | NVRLFNGRDKDSTNFILGDEFAVLRFYRNGESISYIAYKEAQMM | |
| | NEIAEFYAAPFKKTRAINEKEAFECIYDSRTRSAGKYPVSVKINV | |
| | DKAKKILNLPECDYINDYIKTPQVPHGITESQTRAVPSEPKTVYVI | |
| | CLRENGSTVYPNEVSAQMQDAANSVYAVHGLKRYVNLHFVLY | |
| | TTEYACPSGNADEGLDGFTASLKANPKAEGYDDQIYFLIRWGT | |
| | WDNNILGISWLNSYNVNTASDFKASGMSTTQLMYPGVMAHEL | |
| | GHILGANHADDPKDLMYSKYTGYLFHLSEKNMDIIAKNLGWEIA | |
| | DGD | |

E. faecalis and Gelatinase E (GelE)

In some embodiments, the pathogenic bacterial strain is E. faecalis. E. faecalis is a gram-positive commensal bacterium, and may be identified by its 16S RNA sequence (see Table 3, below). For example, in some embodiments, an E. faecalis strain has a 16S RNA sequence that is at least 90%, at least 95%, at least 96%, at least 97%, at least 98%, or at least 99% identical to SEQ ID NO: 5. In some embodiments, a strain of E. faecalis has a 16S RNA sequence that is at least 97% identical to the sequence of SEQ ID NO: 5. In some embodiments, an E. faecalis strain is isolated from a human fecal sample. In some embodiments, an E. faecalis strain is an engineered strain, such as a non-toxigenic E. faecalis strain engineered to express or overexpress GelE.

TABLE 3
Sequence encoding E. faecalis 16S RNA

| | | SEQ ID |
|---|---|---|
| Species | Sequence | NO: |
| E. faecalis | GACGAACGCTGGCGGCGTGCCTAATACATGCAAGTCG | 5 |
| | AACGCTTCTTTCCTCCCGAGTGCTTGCACTCAATTGGA | |
| | AAGAGGAGTGGCGGACGGGTGAGTAACACGTGGGTA | |
| | ACCTACCCATCAGAGGGGGATAACACTTGGAAACAGG | |
| | TGCTAATACCGCATAACAGTTTATGCCGCATGGCATAA | |
| | GAGTGAAAGGCGCTTTCGGGTGTCGCTGATGGATGGA | |
| | CCCGCGGTGCATTAGCTAGTTGGTGAGGTAACGGCTC | |
| | ACCAAGGCCACGATGCATAGCCGACCTGAGAGGGTGA | |
| | TCGGCCACACTGGGACTGAGACACGGCCCAGACTCCT | |
| | ACGGGAGGCAGCAGTAGGGAATCTTCGGCAATGGACG | |
| | AAAGTCTGACCGAGCAACGCCGCGTGAGTGAAGAAGG | |
| | TTTTCGGATCGTAAAACTCTGTTGTTAGAGAAGAACAA | |
| | GGACGTTAGTAACTGAACGTCNCCTGACGGTATCTAAC | |
| | CAGAAAGCCACGGCTAACTACGTGCCAGCAGCCGCGG | |
| | TAATACGTAGGTGGCAAGCGTTGTCCGGATTTATTGGG | |
| | CGTAAAGCGAGCGCAGGCGGTTTCTTAAGTCTGATGT | |
| | GAAAGCCCCCGGCTCAACCGGGGAGGGTCATTGGAAA | |
| | CTGGGAGACTTGAGTGCAGAAGAGGAGAGTGGAATTC | |
| | CATGTGTAGCGGTGAAATGCGTAGATATATGGAGGAA | |
| | CACCAGTGGCGAAGGCGGCTCTCTGGTCTGTAACTGA | |
| | CGCTGAGGCTCGAAAGCGTGGGGAGCAAACAGGATTA | |
| | GATACCCTGGTAGTCCACGCCGTAAACGATGAGTGCT | |
| | AAGTGTTGGAGGGTTTCCGCCCTTCAGTGCTGCAGCA | |
| | AACGCATTAAGCACTCCGCCTGGGGAGTACGACCGCA | |
| | AGGTTGAAACTCAAAGGAATTGACGGGGGCCCGCACA | |
| | AGCGGTGGAGCATGTGGTTTAATTCGAAGCAACGCGA | |
| | AGAACCTTACCAGGTCTTGACATCCTTTGACCACTCTA | |
| | GAGATAGAGCTTTCCCTTCGGGGACAAAGTGACAGGT | |
| | GGTGCATGGTTGTCGTCAGCTCGTGTCGTGAGATGTT | |
| | GGGTTAAGTCCCGCAACGAGCGCAACCCTTATTGTTA | |
| | GTTGCCATCATTTAGTTGGGCACTCTAGCGAGACTGCC | |
| | GGTGACAAACCGGAGGAAGGTGGGGATGACGTCAAAT | |
| | CATCATGCCCCTTATGACCTGGGCTACACACGTGCTAC | |
| | AATGGGAAGTACAACGAGTCGCTAGACCGCGAGGTCA | |
| | TGCAAATCTCTTAAAGCTTCTCTCAGTTCGGATTGCAG | |
| | GCTGCAACTCGCCTGCATGAAGCCGGAATCGCTAGTA | |
| | ATCGCGGATCAGCACGCCGCGGTGAATACGTTCCCGG | |
| | GCCTTGTACACACCGCCCGTCACACCACGAGAGTTTG | |
| | TAACACCCGAAGTCGGTGAGGTAACCTTTTTGGAGCCA | |
| | GCCGCCTAAGGTGGGATAGATGATTGG | |

E. faecalis strains frequently harbor a gene encoding the enzyme Gelatinase E or GelE. GelE is a virulence factor of E. faecalis. It may contribute to the survival of bacteria in various host tissues, and has been shown enhance biofilm formation in vitro.

GelE is a 30-kDa metalloprotease secreted from E. faecalis strains and is capable of hydrolyzing gelatin, collagen, casein, hemoglobin, and other peptides. An illustrative sequence of GelE is shown in Table 4, below. As will be understood by those of skill in the art, many different variants of GelE are known, for example as shown in Uniprot Accession No. Q833V7.

TABLE 4
GelE amino acid sequence

| Name | Sequence | SEQ ID No: |
|---|---|---|
| GelE | MMKGNKILYILGTGIFVGSSCLFSSLFVAAEEQVYSESEVSTVLS | 6 |
| | KLEKEAISEAAAEQYTVVDRKEDAWGMKHLKLEKQTEGVTVDS | |
| | DNVIIHLDRNGAVTSVTGNPVDQVVKIQSVDAIGEEGVKKIIASD | |
| | NPETKDLVFLAIDKRVNNEGQLFYKVRVTSSPTGDPVSLVYKVN | |
| | ATDGTIMEKQDLTEHVGSEVTLKNSFQVAFNVPVEKSNTGIALH | |
| | GTDNTGVYHAVVDGKNNYSIIQAPSLVALNQNAVDAYTHGKFV | |
| | KTYYEDHFQRHSIDDRGMPILSVVDEQHPDAYDNAFWDGKAM | |
| | RYGETSTPTGKTYASSLDVVGHEMTHGVTEHTAGLEYLGQSG | |
| | ALNESYSDLMGYIISGASNPEIGADTQSVDRKTGIRNLQTPSKH | |
| | GQPETMAQYDDRARYKGTPYYDQGGVHYNSGIINRIGYTIIQNL | |
| | GIEKAQTIFYSSLVNYLTPKAQFSDARDAMLAAAKVQYGDEAAS | |
| | WVSAAFNSAGIGAKEDIQVNQPSESVLVNE | |

C. perfringens and Collagenase A (ColA)

In some embodiments, the pathogenic bacterial strain is C. perfringens. C. perfringens is a spore-forming gram-positive bacterium that is found in many environmental sources as well as in the intestines of humans and animals. C. perfringens may be identified by its 16S RNA sequence (see Table 5, below). For example, in some embodiments, a strain of C. perfringens has a sequence that is at least 90%, at least 95%, at least 96%, at least 97%, at least 98%, or at least 99% identical to the sequence of SEQ ID NO: 7. In some embodiments, a strain of C. perfringens has a 16S RNA sequence that is at least 97% identical to the sequence of SEQ ID NO: 7. In some embodiments, a C. perfringens strain is isolated from a human fecal sample. In some embodiments, a C. perfringens strain is an engineered strain, such as a non-toxigenic C. perfringens strain engineered to express or overexpress ColA.

TABLE 5
Sequence encoding C. perfringens 16S RNA

| | | SEQ ID |
|---|---|---|
| Species | Sequence | NO: |
| C. | TAAATTGAGAGTTTGATCCTGGCTCAGGATGAACGCTG | 7 |
| perfringens | GCGGCGTGCTTAACACATGCAAGTCGAGCGATGAAGT | |
| | TTCCTTCGGGAAACGGATTAGCGGCGGACGGGTGAGT | |
| | AACACGTGGGTAACCTGCCTCATAGAGTGGAATAGCC | |
| | TTCCGAAAGGAAGATTAATACCGCATAACGTTGAAAGA | |
| | TGGCATCATCATTCAACCAAAGGAGCAATCCGCTATGA | |
| | GATGGACCCGCGGCGCATTAGCTAGTTGGTGGGGTAA | |
| | CGGCCTACCAAGGCGACGATGCGTAGCCGACCTGAGA | |
| | GGGTGATCGGCCACATTGGGACTGAGACACGGCCCA | |
| | GACTCCTACGGGAGGCAGCAGTGGGGAATATTGCACA | |
| | ATGGGGGAAACCCTGATGCAGCAACGCCGCGTGAGTG | |
| | ATGAAGGTTTTCGGATCGTAAAGCTCTGTCTTTGGGGA | |
| | AGATAATGACGGTACCCAAGGAGGAAGCCACGGCTAA | |
| | CTACGTGCCAGCAGCCGCGGTAATACGTAGGTGGCGA | |
| | GCGTTATCCGGATTTACTGGGCGTAAAGGGAGCGTAG | |
| | GCGGATGATTAAGTGGGATGTGAAATACCCGGGCTCA | |
| | ACTTGGGTGCTGCATTCCAAACTGGTTATCTAGAGTGC | |
| | AGGAGAGGAGAGTGGAATTCCTAGTGTAGCGGTGAAA | |
| | TGCGTAGAGATTAGGAAGAACACCAGTGGCGAAGGCG | |
| | ACTCTCTGGACTGTAACTGACGCTGAGGCTCGAAAGC | |
| | GTGGGGAGCAAACAGGATTAGATACCCTGGTAGTCCA | |
| | CGCCGTAAACGATGAATACTAGGTGTGGGGGTTTCAA | |
| | CACCTCCGTGCCGCCGCTAACGCATTAAGTATTCCGC | |
| | CTGGGGAGTACGGTCGCAAGATTAAAACTCAAAGGAA | |
| | TTGACGGGGACCCGCACAAGTAGCGGAGCATGTGGTT | |
| | TAATTCGAAGCAACGCGAAGAACCTTACCTACACTTGA | |
| | CATCCCTTGCATTACTCTTAATCGAGGAAATCCCTTCG | |
| | GGGACAAGGTGACAGGTGGTGCATGGTTGTCGTCAGC | |
| | TCGTGTCGTGAGATGTTGGGTTAAGTCCCGCAACGAG | |
| | CGCAACCCTTGTCGTTAGTTACTACCATTAAGTTGAGG | |
| | ACTCTAGCGAGACTGCCTGGGTTAACCAGGAGGAAGG | |
| | TGGGGATGACGTCAAATCATCATGCCCCTTATGTGTAG | |
| | GGCTACACACGTGCTACAATGGCTGGTACAGAGAGAT | |
| | GCAATACCGCGAGGTGGAGCCAAACTTAAAAACCAGT | |
| | CTCAGTTCGGATTGTAGGCTGAAACTCGCCTACATGAA | |
| | GCTGGAGTTACTAGTAATCGCGAATCAGAATGTCGCG | |
| | GTGAATACGTTCCCGGGTCTTGTACACACCGCCCGTC | |
| | ACACCATGAGAGTTGGCAATACCCGAAGTCCGTGAGC | |
| | TAACCGCAAGGAGGCAGCGGCCGAAGGTAGGGTCAG | |
| | CGATTGGGGTGAAGTCGTAACAAGGTAGCCGTAGGAG | |
| | AACCTGCGGCTGGATCACCTCCTTT | |

C. perfringens strains typically harbor a gene encoding the enzyme Collagenase A or COLA. COLA is a toxin that degrades collagen. COLA plays a role in the virulence of C. perfringens, by spreading toxins in cells to host tissue. ColA secretion can also be triggered by pro-inflammatory cytokines during a normal immune response, which can lead to tissue damage. COLA is closely related to, and has similar activity to Collagenase H, an enzyme produced by C. histolyticum. Specifically, COLA and ColH bath digest collagen, have a high degree of homology in the catalytic domain, and have structural similarity (based on 3D in silico modeling).

An illustrative sequence of ColA and ColH is shown in Table 6, below. As will be understood by those of skill in the art, many different variants of these enzymes are known, for example as shown in Uniprot Accession Nos. Q46173 and Q46085.

TABLE 6
ColA and ColH amino acid sequence

| Name | Sequence | SEQ ID No: |
|---|---|---|
| ColA | MKKNLKRGELTKLKLVERWSATFTLAAFILFNSSFKVLAADKKV | 8 |
| | ENSNNGQITREINADQISKTELNNEVATDNNRPLGPSIAPSRAR | |
| | NNKIYTFDELNRMNYSDLVELIKTISYENVPDLFNFNDGSYTFFS | |
| | NRDRVQAIIYGLEDSGRTYTADDDKGIPTLVEFLRAGYYLGFYN | |
| | KQLSYLNTPQLKNECLPAMKAIQYNSNFRLGTKAQDGVVEALG | |
| | RLIGNASADPEVINNCIYVLSDFKDNIDKYGSNYSKGNAVFNLM | |
| | KGIDYYTNSVIYNTKGYDAKNTEFYNRIDPYMERLESLCTIGDKL | |
| | NNDNAWLVNNALYYTGRMGKFREDPSISQRALERAMKEYPYL | |
| | SYQYIEAANDLDLNFGGKNSSGNDIDFNKIKADAREKYLPKTYT | |
| | FDDGKFVVKAGDKVTEEKIKRLYWASKEVKAQFMRVVQNDKA | |
| | LEEGNPDDILTVVIYNSPEEYKLNRIINGFSTDNGGIYIENIGTFFT | |
| | YERTPEESIYTLEELFRHEFTHYLQGRYVVPGMWGQGEFYQE | |
| | GVLTWYEEGTAEFFAGSTRTDGIKPRKSVTQGLAYDRNNRMS | |
| | LYGVLHAKYGSWDFYNYGFALSNYMYNNNMGMFNKMTNYIKN | |
| | NDVSGYKDYIASMSSDYGLNDKYQDYMDSLLNNIDNLDVPLVS | |
| | DEYVNGHEAKDINEITNDIKEVSNIKDLSSNVEKSQFFTTYDMR | |
| | GTYVGGRSQGEENDWKDMNSKLNDILKELSKKSWNGYKTVTA | |
| | YFVNHKVDGNGNYVYDVVFHGMNTDTNTDVHVNKEPKAVIKS | |
| | DSSVIVEEEINFDGTESKDEDGEIKAYEWDFGDGEKSNEAKAT | |
| | HKYNKTGEYEVKLTVTDNNGGINTESKKIKVVEDKPVEVINESE | |
| | PNNDFEKANQIAKSNMLVKGTLSEEDYSDKYYFDVAKKGNVKI | |
| | TLNNLNSVGITWTLYKEGDLNNYVLYATGNDGTVLKGEKTLEP | |
| | GRYYLSVYTYDNQSGTYTVNVKGNLKNEVKETAKDAIKEVENN | |
| | NDFDKAMKVDSNSKIVGTLSNDDLKDIYSIDIQNPSDLNIVVENL | |
| | DNIKMNWLLYSADDLSNYVDYANADGNKLSNTCKLNPGKYYLC | |
| | VYQFENSGTGNYIVNLQNK | |
| ColH | MKRKCLSKRLMLAITMATIFTVNSTLPIYAAVDKNNATAAVQNE | 9 |
| | SKRYTVSYLKTLNYYDLVDLLVKTEIENLPDLFQYSSDAKEFYG | |
| | NKTRMSFIMDEIGRRAPQYTEIDHKGIPTLVEVVRAGFYLGFHN | |
| | KELNEINKRSFKERVIPSILAIQKNPNFKLGTEVQDKIVSATGLLA | |
| | GNETAPPEVVNNFTPILQDCIKNIDRYALDDLKSKALFNVLAAPT | |
| | YDITEYLRATKEKPENTPWYGKIDGFINELKKLALYGKINDNNS | |
| | WIIDNGIYHIAPLGKLHSNNKIGIETLTEVMKVYPYLSMQHLQSA | |
| | DQIKRHYDSKDAEGNKIPLDKFKKEGKEKYCPKTYTFDDGKVII | |
| | KAGARVEEEKVKRLYWASKEVNSQFFRVYGIDKPLEEGNPDDI | |
| | LTMVIYNSPEEYKLNSVLYGYDTNNGGMYIEPEGTFFTYEREA | |
| | QESTYTLEELFRHEYTHYLQGRYAVPGQWGRTKLYDNDRLTW | |
| | YEEGGAELFAGSTRTSGILPRKSIVSNIHNTTRNNRYKLSDTVH | |
| | SKYGASFEFYNYACMFMDYMYNKDMGILNKLNDLAKNNDVDG | |
| | YDNYIRDLSSNYALNDKYQDHMQERIDNYENLTVPFVADDYLV | |
| | RHAYKNPNEIYSEISEVAKLKDAKSEVKKSQYFSTFTLRGSYTG | |
| | GASKGKLEDQKAMNKFIDDSLKKLDTYSWSGYKTLTAYFTNYK | |
| | VDSSNRVTYDVVFHGYLPNEGDSKNSLPYGKINGTYKGTEKEK | |
| | IKFSSEGSFDPDGKIVSYEWDFGDGNKSNEENPEHSYDKVGTY | |
| | TVKLKVTDDKGESSVSTTTAEIKDLSENKLPVIYMHVPKSGALN | |
| | QKVVFYGKGTYDPDGSIAGYQWDFGDGSDFSSEQNPSHVYTK | |
| | KGEYTVTLRVMDSSGQMSEKTMKIKITDPVYPIGTEKEPNNSKE | |
| | TASGPIVPGIPVSGTIENTSDQDYFYFDVITPGEVKIDINKLGYG | |
| | GATWVVYDENNNAVSYATDDGQNLSGKFKADKPGRYYIHLYM | |
| | FNGSYMPYRINIEGSVGR | |

Provided herein are compounds that may bind to and/or inhibit BFT, COLA, and/or GelE. In some embodiments, the compounds of the disclosure bind to BFT, COLA, and/or GelE with an inhibition constant in the range of about 10⁻⁵ to about 10⁻¹³ M, e.g., about 10⁻⁵M, about 10⁻⁶M, about 10⁻⁷ M, about 10⁻⁸ M, about 10⁻⁹ M, about 10⁻¹⁰ M, about 10⁻¹¹ M, about 10⁻¹² M, or about 10⁻¹³ M, including all ranges and values therebetween. In some embodiments, the BFT comprises the amino acid sequence of any one of SEQ ID NO: 2-4. In some embodiments, the BFT comprises an amino acid sequence that is at least 95%, at least 96%, at least 97%, at least 98%, or at least 99% identical to the sequence of any one of SEQ ID NO: 2-4. In some embodiments, the small molecules bind to and/or inhibit at least one of BFT1, BFT2, and BFT3. In some embodiments, the small molecules bind to and/or inhibit BFT1 and BFT2. In some embodiments, the small molecules bind to and/or inhibit BFT1 and BFT3. In some embodiments, the small molecules bind to and/or inhibit BFT2 and BFT3. In some embodiments, the small molecules bind to and/or inhibit BFT1, BFT2, and BFT3.

In some embodiments, the GelE comprises the amino acid sequence of SEQ ID NO: 6. In some embodiments, the GelE comprises an amino acid sequence that is at least 95%, at least 96%, at least 97%, at least 98%, or at least 99% identical to the sequence of SEQ ID NO: 6. In some embodiments, the small molecules bind to and/or inhibit GelE.

In some embodiments, the ColA comprises the amino acid sequence of SEQ ID NO: 8. In some embodiments, the ColA comprises an amino acid sequence that is at least 95%, at least 96%, at least 97%, at least 98%, or at least 99% identical to SEQ ID NO: 8. In some embodiments, the small molecules bind to and/or inhibit ColA. In some embodiments, the small molecules bind to and/or inhibit ColA.

In some embodiments, the small molecules bind and/or inhibit one, two or all three of BFT, GelE, and ColA. For example, in some embodiments, small molecules bind and/or inhibit only BFT. In some embodiments, the small molecules bind and/or inhibit only GelE. In some embodiments, the small molecules bind and/or inhibit only ColA. In some embodiments, the small molecules bind and/or inhibit BFT and GelE. In some embodiments, the small molecules bind and/or inhibit BFT and ColA. In some embodiments, the small molecules bind and/or inhibit ColA and GelE. In some embodiments, the small molecules bind and/or inhibit BFT, GelE, and ColA. In some embodiments, a small molecule binds to each of ColA, GelE, and BFT with similar affinity. In some embodiments, a small molecule binds to each of ColA, GelE, and BFT with different affinity. In some embodiments, a small molecule inhibits the activity of each of ColA, GelE, and BFT to a different extent. In some embodiments, a small molecule inhibits the activity of each of ColA, GelE, and BFT to an approximately equal extent.

The small molecules of the present disclosure may bind to and/or inhibit BFT, ColA and/or GelE in vitro, or in vivo. In some embodiments, the small molecules bind to and/or inhibit BFT, ColA and/or GelE that is bound to a cell membrane. In some embodiments, the small molecules bind to and/or inhibit secreted BFT, ColA and/or GelE. In some embodiments, the small molecules bind to and/or inhibit intracellular BFT, ColA and/or GelE.

In some embodiments, the small molecules of the present disclosure decrease BFT, ColA, and/or GelE activity by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, the small molecule inhibitors decrease BFT, ColA, and/or GelE activity by about 5% to about 25%, about 25% to about 50%, about 50% to about 75%, or about 75% to 100%. In some embodiments, the small molecule inhibitors decrease BFT, ColA, and/or GelE activity by about 95% to 100%, e.g., a decrease in activity of 95%, 96%, 97%, 98%, 99%, or 100%.

In some embodiments, the small molecules of the present disclosure diminish the pathogenic effects of a strain of B. fragilis (ETBF) expressing a BFT toxin, a strain of E. faecalis expressing the gelatinase GelE, or a strain of C. perfringens expressing ColA. In some embodiments, the small molecules of the present disclosure substantially eliminate the pathogenic effects of a strain of B. fragilis (ETBF) expressing a BFT toxin, a strain of E. faecalis expressing the gelatinase GelE, or a strain of C. perfringens. In some embodiments, the small molecules of the present disclosure completely eliminate the pathogenic effects of a strain of B. fragilis (ETBF) expressing a BFT toxin, a strain of E. faecalis expressing the gelatinase GelE, or a strain of C. perfringens.

In some embodiments, the inhibitor binds to and inhibits the activity of a BFT. In some embodiments, the inhibitor reduces the ability of a BFT to release E-cadherin from a cell. For example, an inhibitor may reduce E-cadherin release by at least about 5%, at least about 10%, at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, the inhibitor reduces the ability of a BFT to cause secretion of IL-8 from a cell. For example, an inhibitor may decrease BFT-mediated IL-8 secretion by at least about 5%, at least about 10%, at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, or at least about 95%.

In some embodiments, the compound inhibits BFT, ColA, and/or GelE by competitive inhibition. In some embodiments, the inhibitor inhibits BFT, ColA, and/or GelE by non-competitive inhibition. In some embodiments, the inhibitor inhibits BFT, ColA, and GelE by uncompetitive inhibition. In some embodiments, the inhibitor inhibits BFT, ColA, and/or GelE by mixed inhibition (e.g., allosteric inhibition). The inhibition may be reversible, or may be irreversible.

In some embodiments, the compound of the present disclosure has the structure of Formula I:

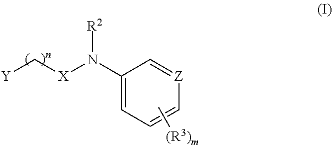

or a stereoisomer or a pharmaceutically acceptable salt thereof, wherein:
X is —C(O)— or —S(O)₂—;
Y is —SC(O)R¹ or —SH;
Z is CR⁹ or N;
R¹ is alkyl, haloalkyl, or —NR⁴R⁵;
R² is H, alkyl, or cycloalkyl;
R³ is —OH, alkoxy, —C(O)R⁶, or —C(O)N(H)-alkylene-C(O)N⁷R⁸, wherein the alkylene is optionally substituted;
R⁴, R⁵, R⁷, and R⁸ are each independently H, alkyl, cycloalkyl, —CH₂cycloalkyl, aryl, —CH₂aryl, or —CH₂heteroaryl;
R⁶ is alkyl, alkoxy, or —N(H)alkyl;
R⁹ is H, halogen, C₁₋₅alkyl, haloalkyl, or alkoxy;
m is 0, 1, 2, or 3; and
n is 1 or 2.

In some embodiments of Formula I, X is —C(O)—. In some embodiments, X is —S(O)₂—.

In some embodiments of Formula I, Y is —SC(O)R¹. In some embodiments, Y is —SH.

In some embodiments of Formula I, Z is CR⁹. In some embodiments, Z is N.

In some embodiments of Formula I, R¹ is C₁₋₅alkyl or —NR⁴R⁵. In some embodiments, R¹ is C₁₋₅alkyl. In some embodiments, R¹ is —NR⁴R⁵. In some embodiments, the C₁₋₅alkyl is methyl, ethyl, propyl, isopropyl, or t-butyl.

In some embodiments of Formula I, R² is H, C₁₋₅alkyl or C₃₋₆cycloalkyl. In some embodiments, R² is H. In some embodiments, R² is C₁₋₅alkyl. In some embodiments, R² is C₃₋₆cycloalkyl. In some embodiments, the C₁₋₅alkyl is methyl, ethyl, propyl, isopropyl, butyl, t-butyl, s-butyl, or isoamyl.

In some embodiments, R³ is —OH, —C₁₋₅alkoxy, —C₁₋₅haloalkoxy, —C(O)C₁₋₅alkyl, —C(O)OC₁₋₅alkyl, or —C(O)N(H)—(C₁₋₃alkylene)-C(O)N⁷R⁸, wherein the alkylene is optionally substituted with an alkyl, cycloalkyl, cycloalkylalkyl, aralkyl, or heteroaralkyl. In some embodiments, R³ is —OH, —C₁₋₅alkoxy, —C₁₋₅haloalkoxy, —C(O)C₁₋₅alkyl, or —C(O)OC₁₋₅alkyl. In some embodiments, R³ is —OH, C₁₋₅alkoxy, —C(O)C₁₋₅alkyl, —C(O)OC₁₋₅alkyl, or —C(O)N(H)—(C₁₋₃alkylene)-C(O)N⁷R⁸, wherein the alkylene is optionally substituted with an alkyl, cycloalkyl, cycloalkylalkyl, aralkyl, or heteroaralkyl. In some embodiments of Formula I, R³ is alkoxy or —C(O)R⁶. In some embodiments, R³—OH, —OMe, —C(O)CH₃, —C(O)OCH₃, or —C(O)N(H)—CH(CH₂Ph)-C(O)NHMe. In some embodiments, R³ is —C(O)N(H)—(C₁₋₃alkylene)-C(O)N⁷R⁸ wherein the alkylene is optionally substituted with an alkyl, cycloalkyl, cycloalkylalkyl, aralkyl, or heteroaralkyl. In some embodiments, the C₁₋₅alkyl is methyl, ethyl, propyl, isopropyl, butyl, t-butyl, s-butyl, or isoamyl. In some embodiments, the C₁₋₅alkyl is methyl. In some embodiments, the C₁₋₅alkoxy is —OMe, —OEt, —OPr, OiPr, —OBu, or —OtBu. In some embodiments, the cycloalkyl is a C₃₋₆cycloalkyl. In some embodiments, the cycloalkyl is a cyclopropyl.

In some embodiments of Formula I, R⁴ and R⁵ are each independently H, C₁₋₅alkyl, C₃₋₆cycloalkyl, —CH₂—(C₃₋₆cycloalkyl), Ph, —CH₂Ph, or —CH₂heteroaryl. In some embodiments of Formula I, R⁴ and R⁵ are each independently H or C₁₋₅alkyl. In some embodiments, the C₁₋₅alkyl is methyl, ethyl, propyl, isopropyl, butyl, t-butyl, s-butyl, or isoamyl. In some embodiments, the heteroaryl is a 5- or 6-membered heteroaryl having 1, 2, or 3 heteroatoms selected from the group consisting of N, O, and S.

In some embodiments of Formula I, R⁶ is C₁₋₅alkyl, C₁₋₅alkoxy, or —N(H)C₁₋₅alkyl. In some embodiments, the C₁₋₅alkyl is methyl, ethyl, propyl, isopropyl, butyl, t-butyl, s-butyl, or isoamyl. In some embodiments, the C₁₋₅alkyl is methyl. In some embodiments, the C₁₋₅alkoxy is —OMe, —OEt, —OPr, —OiPr, —OBu, or —OtBu.

In some embodiments of Formula I, R⁷ and R⁸ are each independently H, C₁₋₅alkyl, —CH₂aryl, or —CH₂heteroaryl. In some embodiments, R⁷ and R⁸ are each independently H or C₁₋₅alkyl. In some embodiments, the C₁₋₅alkyl is methyl, ethyl, propyl, isopropyl, butyl, t-butyl, s-butyl, or isoamyl. In some embodiments, the C₁₋₅alkyl is methyl. In some embodiments, the C₁₋₅alkoxy is —OMe, —OEt, —OPr, —OiPr, —OBu, or —OtBu. In some embodiments, the heteroaryl is a 5- or 6-membered heteroaryl having 1, 2, or 3 heteroatoms selected from the group consisting of N, O, and S. In some embodiments, the aryl is a phenyl.

In some embodiments of Formula I, R⁹ is H, halogen, C₁₋₅alkyl, C₁₋₂ haloalkyl, or C₁₋₅alkoxy. In some embodiments, the C₁₋₅alkyl is Me, Et, Pr, or Pr. In some embodiments, the C₁₋₅alkoxy is —OMe, —OEt, or —OiPr. In some embodiments, the C₁₋₂ haloalkyl is CF₃, CHF₂, or CH₂F.

In some embodiments of Formula I, m is 1, 2, or 3. In some embodiments, m is 1 or 2. In some embodiments, m is 1. In some embodiments, m is 2.

In some embodiments of Formula I, n is 1. In some embodiments of Formula I, n is 2.

In some embodiments, the present disclosure provides a compound of Formula II or Formula IIA:

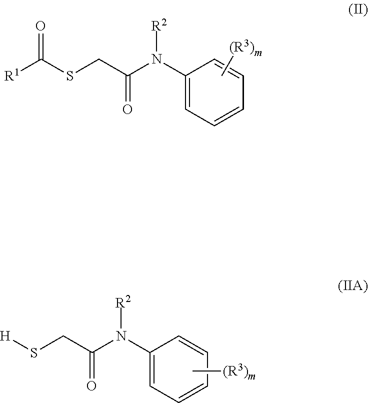

or a stereoisomer or a pharmaceutically acceptable salt thereof, wherein R¹, R², R³, and m are as defined above in Formula I.

In some embodiments, the present disclosure provides a compound of Formula III:

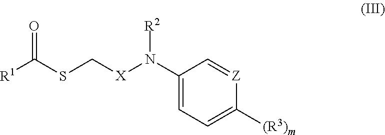

or a stereoisomer or a pharmaceutically acceptable salt thereof, wherein X, Z, R¹, R², R³, and m are as defined above in Formula I.

In some embodiments, the present disclosure provides a compound of Formula IV:

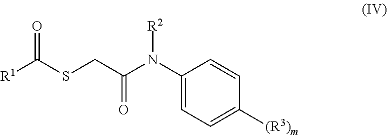

or a stereoisomer or a pharmaceutically acceptable salt thereof, wherein R¹, R², R³, and m are as defined above in Formula I.

In some embodiments, the present disclosure provides a compound of Formula V:

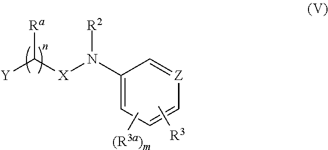

or a stereoisomer or a pharmaceutically acceptable salt thereof, wherein:
X is —C(O)— or —S(O)₂—;
Y is —SC(O)R¹ or —SH;
Z is CR⁹ or N;
R^{a} is H, alkyl, haloalkyl, aryl, alkylene-cycloalkyl, alkylene-heterocycloalkyl, heteroaryl, alkylene-aryl, or alkylene-heteroaryl;
R¹ is alkyl, haloalkyl, or —NR⁴R⁵;
R² is H, alkyl, or cycloalkyl;
R³ is —OH, alkoxy, —O-alkylene-NR⁷R⁸, —C(O)R⁸, or —C(O)N(H)-alkylene-C(O)NR⁷R⁸, wherein the alkylene is optionally substituted;
R³ is alkyl, halogen, alkoxy, haloalkyl, or haloalkoxy;
R⁴, R⁵, R⁷, and R⁸ are each independently H, alkyl, cycloalkyl, —CH₂cycloalkyl, aryl, or —CH₂aryl;
R⁸ is alkyl, haloalkyl, alkoxy, or —N(H)alkyl;
m is 0, 1, 2, or 3; and
n is 1 or 2.

In some embodiments of Formula V, X is —C(O)—. In some embodiments, X is —S(O)₂—.

In some embodiments of Formula V, Y is —SC(O)R¹. In some embodiments, Y is —SH.

In some embodiments of Formula V, Z is CR⁹. In some embodiments, Z is N.

In some embodiments of Formula V, R^{a} is H, halogen, alkyl, haloalkyl, alkoxy, aryl, alkylene-cycloalkyl, alkylene-heterocycloalkyl, heteroaryl, alkylene-aryl, or alkylene-heteroaryl. In some embodiments, R^{a} is alkyl, haloalkyl, aryl, alkylene-cycloalkyl, alkylene-heterocycloalkyl, heteroaryl, alkylene-aryl, or alkylene-heteroaryl. In some embodiments, R^{a} is H, alkyl, haloalkyl, or alkylene-cycloalkyl. In some embodiments, R^{a} is alkyl, haloalkyl, or alkylene-cycloalkyl. In some embodiments, R^{a} is H, halogen, alkyl, alkylene-cycloalkyl, alkylene-heterocycloalkyl, alkylene-aryl, or alkylene-heteroaryl. In some embodiments, R^{a} is halogen, alkyl, alkylene-cycloalkyl, alkylene-heterocycloalkyl, alkylene-aryl, or alkylene-heteroaryl. In some embodiments, Rₐ is H, alkyl, or haloalkyl. In some embodiments, R^{a} is alkyl or haloalkyl. In some embodiments, R^{a} is H or alkyl. In some embodiments, R^{a} is alkyl. In some embodiments, the alkyl is a C₁₋₅alkyl. In some embodiments, the C₁₋₅alkyl is methyl, ethyl, n-propyl, isopropyl, n-butyl, isobutyl, sec-butyl, isoamyl, or neopentyl. In some embodiments, the C₁₋₅alkyl is methyl, ethyl, n-propyl, n-butyl, isobutyl, sec-butyl, isoamyl, or neopentyl. In some embodiments, the C₁₋₅alkyl is methyl, ethyl, n-propyl, or isopropyl. In some embodiments, the C₁₋₅alkyl is methyl, ethyl, or n-propyl. In some embodiments, the C₁₋₅alkyl is ethyl. In some embodiments, the C₁₋₅alkyl is optionally substituted with one or more halogen, —OH, alkoxy, thioalkyl, or aminoalkyl groups. In some embodiments, R^{a} is a C₂₋₅alkyl. In some embodiments, the C₂₋₅alkyl is ethyl, n-propyl, isopropyl, n-butyl, isobutyl, sec-butyl, isoamyl, or neopentyl. In some embodiments, C₂₋₅alkyl is ethyl, n-propyl, n-butyl, isobutyl, sec-butyl, isoamyl, or neopentyl. In some embodiments, the C₂₋₅alkyl is ethyl, n-propyl, or isopropyl. In some embodiments, the C₂₋₅alkyl is ethyl, or n-propyl. In some embodiments, the C₂₋₅alkyl is ethyl. In some embodiments, the C₂₋₅alkyl is optionally substituted with one or more halogen, —OH, alkoxy, thioalkyl, or aminoalkyl groups. In some embodiments, the halogen is F. In some embodiments, the haloalkyl is selected from CF₃, CH₂CF₃, CF₂CH₃, CHF₂, or CH₂F. In some embodiments, the cycloalkyl is a C₃₋₆cycloalkyl. In some embodiments, the aryl is a phenyl. In some embodiments, the heteroaryl is a 5- or 6-membered heteroaryl having 1, 2, or 3 heteroatoms selected from N, O, and S. In some embodiments, the heterocyclyl is a 4- to 7-member heterocyclyl with 1 or 2 heteroatoms selected from N, O, and S. In some embodiments, the alkylene is a C₁₋₅alkylene. In some embodiments, the alkylene is a C₁₋₃alkylene. In some embodiments, the alkylene is methylene (—CH₂—) or ethylene (—CH₂—CH₂—). In some embodiments, the alkylene is methylene. In some embodiments, R^{a} is —CH₂CH₃, —CH₂CH₃, or —CH₂CF₃.

In some embodiments of Formula V, R¹ is C₁₋₅alkyl or —NR⁴R⁵. In some embodiments, R¹ is C₁₋₅alkyl. In some embodiments, R¹ is C₁₋₃alkyl. In some embodiments, R¹ is —NR⁴R⁵. In some embodiments, the C₁₋₅alkyl is methyl, ethyl, propyl, isopropyl, or t-butyl. In some embodiments, —NR⁴R⁵ is —NH₂, —NH(Me), —NH(i-Pr), —NH(t-Bu), —N(CH₃)₂, or

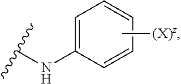

wherein each X is independently halogen or haloalkyl and z is 0, 1, 2, or 3. In some embodiments, each X is independently Cl or F and z is 0, 1, 2, or 3. In some embodiments, the

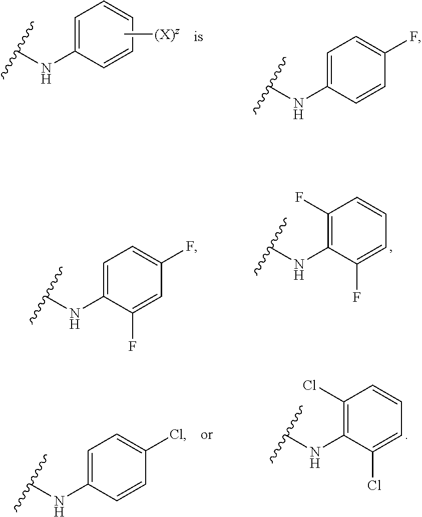

In some embodiments of Formula V, R² is H, C₁₋₅alkyl or C₃₋₆cycloalkyl. In some embodiments, R² is H. In some embodiments, R² is C₁₋₅alkyl. In some embodiments, R² is C₃₋₆cycloalkyl. In some embodiments, the C₁₋₅alkyl is methyl, ethyl, propyl, isopropyl, butyl, t-butyl, s-butyl, or isoamyl.

In some embodiments, R^{a} is alkyl and R² is H. In some embodiments, R^{a} is C₁₋₅alkyl and R² is H. In some embodiments, R^{a} is C₂₋₅alkyl and R² is H. In some embodiments, R⁶ is Et and R² is H. In some embodiments, n is 1.

In some embodiments of Formula V, R³ is —OH, alkoxy, —O-alkylene-OR⁷, —O-alkylene-SR⁷, —O-alkylene-NR⁷R⁸, —C(O)R⁸, or —C(O)N(H)-alkylene-C(O)NR⁷R⁸. In some embodiments, R³ is alkoxy, —O-alkylene-NR⁷R⁸, —C(O)R⁸, or —C(O)N(H)-alkylene-C(O)NR⁷R⁸. In some embodiments, R³ is alkoxy or —C(O)R⁸. In some embodiments, R³ is —OH or alkoxy. In some embodiments, R³ is alkoxy, —O-alkylene-OR⁷, —O-alkylene-SR⁷, —O-alkylene-NR⁷R⁸, —C(O)R⁸, or —C(O)N(H)-alkylene-C(O)NR⁷R⁸. In some embodiments, R³ is alkoxy, —O-alkylene-NR⁷R⁸, —C(O)R⁸, or —C(O)N(H)-alkylene-C(O)NR⁷R⁸. In some embodiments, —C(O)R⁸ or —O-alkylene-NR⁷R⁸. In some embodiments, R³ is —C(O)R⁶. In some embodiments, the alkoxy is an —O(C₁₋₅alkyl). In some embodiments, the —O(C₁₋₅alkyl) is —OMe, —OEt, —OPr, OiPr, —OBu, or —OtBu. In some embodiments, the —O(C₁₋₅alkyl) is —OMe. In some embodiments, the alkoxy is a haloalkoxy. In some embodiments, the alkoxy is a fluoroalkoxy. In some embodiments, the haloalkoxy is —CF₃, —CH₂CF₃, —CHF₂, or —CH₂F. In some embodiments, the alkylene is a C₁₋₃alkylene. In some embodiments, the alkylene is methylene (—CH₂—) or ethylene (—CH₂—CH₂—). In some embodiments, the alkylene is ethylene. In some embodiments, the alkylene is a C₁₋₃alkylene (e.g., ethylene) optionally substituted with oxo, alkyl, alkylene-cycloalkyl, alkylene-heterocycloalkyl, alkylene-aryl, or alkylene-heteroaryl.

In some embodiments, R³ is —OH, —C₁₋₅alkoxy, —C(O)C₁₋₅alkyl, —C(O)OC₁₋₅alkyl, or —C(O)N(H)(C₁₋₃alkylene)-C(O)N⁷R⁸, wherein the alkylene is optionally substituted with an alkyl, cycloalkyl, cycloalkylalkyl, aralkyl, or heteroaralkyl. In some embodiments, R³—OH, —OMe, —C(O)CH₃, —C(O)OCH3, or —C(O)N(H)—CH(CH₂Ph)-C(O)NHMe. In some embodiments, R³ is —C(O)N(H)—(Ci-3alkylene)-C(O)N⁷R⁸ wherein the alkylene is optionally substituted with an alkyl, cycloalkyl, cycloalkylalkyl, aralkyl, or heteroaralkyl. In some embodiments, the alkyl is a C₁₋₅alkyl. In some embodiments, the C₁₋₅alkyl is methyl, ethyl, propyl, isopropyl, butyl, t-butyl, s-butyl, or isoamyl. In some embodiments, the C₁₋₅alkyl is methyl. In some embodiments, the alkoxy is an —O(C₁₋₅alkyl). In some embodiments, the —C₁₋₅alkoxy is —OMe, —OEt, —OPr, —OiPr, —OBu, or —OtBu. In some embodiments, the —C₁₋₅alkoxy is an —C₁₋₅haloalkoxy. In some embodiments, the —C₁₋₅alkoxy is a —C₁₋₅fluoroalkoxy In some embodiments, the —(C₁₋₅alkoxy) is —OCF₃, —OCF₂H, —OCH₂F, —OCH₂CF₃, or —OCF₂CF₃. In some embodiments, the —C₁₋₅alkoxy is —OMe. In some embodiments, the cycloalkyl is a C₃₋₆cycloalkyl. In some embodiments, the cycloalkyl is a cyclopropyl.

In some embodiments, R³ is —C₁₋₅alkoxy. In some embodiments, the C₁₋₅alkoxy is —OMe, —OEt, —OPr, —OiPr, —OBu, or —OtBu. In some embodiments, the —C₁₋₅alkoxy is a —C₁₋₅haloalkoxy. In some embodiments, the —C₁₋₅alkoxy is a —C₁₋₅fluoroalkoxy In some embodiments, the —C₁₋₅alkoxy is —OCF₃, —OCF₂H, —OCH₂F, —OCH₂CF₃, or —OCF₂CF₃. In some embodiments, the C₁₋₅alkoxy is —OMe, —OEt, —OPr, —OiPr, —OBu, —OtBu, —OCF₃, —OCF₂H, —OCH₂F, —OCH₂CF₃, or —OCF₂CF₃.

In some embodiments, R³ is —C(O)R⁶. In some embodiments, R⁶ is alkyl. In some embodiments, the alkyl is Me, Et, nPr, —CH₂O(C₁₋₅alkyl), —CH₂S(C₁₋₅alkyl), or —CH₂N(H)(C₁₋₅alkyl). In some embodiments, the alkyl is Me. In some embodiments, R⁶ is C₁₋₅alkyl, C₁₋₅alkoxy, or —N(H)C₁₋₅alkyl. In some embodiments, R⁶ is a C₁₋₅alkyl. In some embodiments, the C₁₋₅alkyl is methyl, ethyl, propyl, isopropyl, butyl, t-butyl, s-butyl, or isoamyl. In some embodiments, the C₁₋₅alkyl is methyl. In some embodiments, the C₁₋₅alkoxy is —OMe, —OEt, —OPr, OiPr, —OBu, or —OtBu. In some embodiments, R⁶ is Me, CH₂CF₃, or —OMe. In some embodiments, R⁶ is a C₂₋₅alkyl.

In some embodiments, R³ is —O-alkylene-NR⁷R⁸. In some embodiments, the —O-alkylene-NR⁷R⁸ is

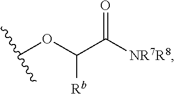

wherein R^{b} is H, alkyl, alkylene-cycloalkyl, alkylene-heterocycloalkyl, alkylene-aryl, or alkylene-heteroaryl. In some embodiments, the alkylene is a C₁₋₃ alkylene. In some embodiments, the alkylene is methylene (—CH₂—) or ethylene (—CH₂—CH₂—). In some embodiments, the alkylene is methylene. In some embodiments, R^{b} is H, —C₁₋₅alkyl, or —CH₂aryl. In some embodiments, the C₁₋₅alkyl is methyl, ethyl, propyl or isopropyl. In some embodiments, the C₁₋₅alkyl is methyl or ethyl. In some embodiments, the C₁₋₅alkyl is methyl. In some embodiments, the —CH₂aryl is —CH₂phenyl.

In some embodiments, R³ is —C(O)N(H)-alkylene-C(O)NR⁷R⁸. In some embodiments, the —C(O)N(H)-alkylene-C(O)NR⁷R⁸ is

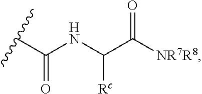

wherein R^{c} is H, alkyl, alkylene-cycloalkyl, alkylene-heterocycloalkyl, alkylene-aryl, or alkylene-heteroaryl. In some embodiments, the alkylene is a C₁₋₃alkylene. In some embodiments, the alkylene is methylene (—CH₂—) or ethylene (—CH₂—CH₂—). In some embodiments, the alkylene is ethylene. In some embodiments, R^{c} is —C₁₋₅alkyl, or —CH₂aryl. In some embodiments, the C₁₋₅alkyl is methyl, ethyl, propyl or isopropyl. In some embodiments, the C₁₋₅alkyl is methyl or ethyl. In some embodiments, the C₁₋₅alkyl is methyl In some embodiments, the —CH₂aryl is —CH₂phenyl.

In some embodiments, R^{a} is alkyl, R² is H, and R³ is

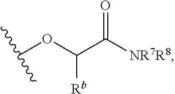

wherein R^{b}, R⁷, and R⁸ are as defined in Formula V. In some embodiments, R^{a} is C₁₋₅alkyl, R² is H, and R³ is

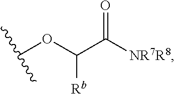

wherein R^{b}, R⁷, and R⁸ are as defined in Formula V.

In some embodiments, R^{a} is Et, R² is H, and R³ is

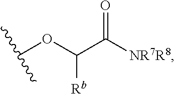

wherein R^{b}, R⁷, and R⁸ are as defined in Formula V. In some embodiments, n is 1.

In some embodiments, R^{a} is alkyl, R² is H, and R³ is —C(O)alkyl. In some embodiments, R^{a} is C₁₋₅alkyl, R² is H, and R³ is —C(O)alkyl. In some embodiments, R^{a} is C₂₋₅alkyl, R² is H, and R³ is —C(O)alkyl. In some embodiments, R^{a} is Et, R² is H, and R³ is —C(O)alkyl. In some embodiments, R³ is —C(O)C₁₋₅alkyl. In some embodiments, R³ is —C(O)C₁₋₃alkyl. In some embodiments, R³ is —C(O)CH₃. In some embodiments, n is 1.

In some embodiments, R^{a} is alkyl, R² is H, and R³ is —C₁₋₅alkoxy, as defined herein. In some embodiments, R^{a} is C₁₋₅alkyl, R² is H, and R³ is —C₁₋₅alkoxy, as defined herein. In some embodiments, R^{a} is Et, R² is H, and R³ is —C₁₋₅alkoxy, as defined herein. In some embodiments, n is 1.

In some embodiments, R^{a} is alkyl, Y is —SC(O)R¹, wherein R¹ is NR⁴R⁵, R² is H, R³ is —C(O)alkyl. In some embodiments, R^{a} is C₁₋₅alkyl, R² is H, and R³ is —C(O)alkyl. In some embodiments, R^{a} is C₂₋₅alkyl, R² is H, and R³ is —C(O)alkyl. In some embodiments, R^{a} is Et, R² is H, and R³ is —C(O)alkyl. In some embodiments, R³ is —C(O)C₁₋₅alkyl. In some embodiments, R³ is —C(O)C₁₋₃alkyl. In some embodiments, R³ is —C(O)CH₃. In some embodiments, n is 1.

In some embodiments, R^{a} is alkyl, R² is H, Y is —SC(O)R¹, wherein R¹ is NR⁴R⁵, and R³ is —C₁₋₅alkoxy, as defined herein. In some embodiments, R^{a} is C₁₋₅alkyl, R² is H, and R³ is —C₁₋₅alkoxy, as defined herein. In some embodiments, R^{a} is Et, R² is H, and R³ is —C₁₋₅alkoxy, as defined herein. In some embodiments, n is 1.

In some embodiments of Formula V, each R^{3a} is independently alkyl, halogen, alkoxy, or haloalkyl. In some embodiments, each R^{3a} is independently C₁₋₅alkyl, halogen, —O(C₁₋₅alkyl), C₁₋₅haloalkyl or —O(C₁₋₅haloalkyl). In some embodiments, each R^{3a} is independently H, halogen, C₁₋₂haloalkyl, or C₁₋₅alkyl or —O(C₁₋₅alkyl). In some embodiments, the C₁₋₅alkyl is methyl, ethyl, propyl, isopropyl, butyl, or t-butyl. In some embodiments, the halogen is F, Br, Cl or I. In some embodiments, the halogen is F or Cl. In some embodiments, the halogen is F. In some embodiments, the haloalkyl is CF₃, CH₂CF₃ CF₂CH₃, CHF₂, or CH₂F. In some embodiments, the haloalkoxy is —OCF₃, —OCH₂CF₃, —OCF₂CH₃, —OCHF₂, or —OCH₂F. In some embodiments, alkoxy is —OMe, —OEt, —OPr, OiPr, —OBu, or —OtBu. In some embodiments, each R^{3a} is independently H, F, Me, i-Pr, CF₃, CF₂H, —CH₂F, or —OMe. In some embodiments, R^{3a} is H.

In some embodiments of Formula V, R⁴ and R⁵ are each independently H, C₁₋₅alkyl, C₃₋₆ecycloalkyl, —CH₂—(C₃₋₆cycloalkyl), Ph, —CH₂Ph, or —CH₂heteroaryl. In some embodiments of Formula V, R⁴ and R⁵ are each independently H, C₁₋₅alkyl, or aryl. In some embodiments of Formula V, R⁴ and R⁵ are each independently H, C₁₋₅alkyl, or phenyl. In some embodiments, the phenyl is optionally substituted with one or more halogen, alkyl, and alkoxy. In some embodiments, the phenyl is optionally substituted with one or more halogen. In some embodiments, the halogen is Cl or F. In some embodiments of Formula V, R⁴ and R⁵ are each independently H or C₁₋₅alkyl. In some embodiments, R⁴ is H and R⁵ is H, C₁₋₅alkyl, C₃₋₆cycloalkyl, —CH₂—(C₃₋₆cycloalkyl), Ph, —CH₂Ph, or —CH₂heteroaryl. In some embodiments of Formula V, R⁴ is H and R⁵ is H, C₁₋₅alkyl, or aryl. In some embodiments of Formula V, R⁴ is H and R⁵ is H, C₁₋₅alkyl, or phenyl. In some embodiments, the phenyl is optionally substituted with one or more halogen, alkyl, or alkoxy. In some embodiments, the phenyl is optionally substituted with one or more halogen. In some embodiments, the halogen is Cl or F. In some embodiments, the optionally substituted phenyl is

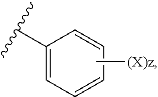

wherein each X is independently F, Br, or Cl; and z is 0, 1, 2, or 3. In some embodiments, the optionally substituted phenyl is

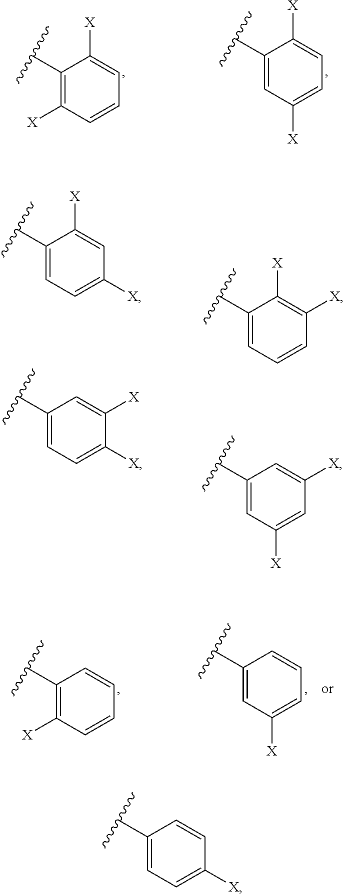

wherein X is each independently F, Br, or Cl; and z is 0, 1, 2, or 3. In some embodiments, each X is each independently Cl or F. In some embodiments, X is Cl. In some embodiments, X is F. In some embodiments, z is 1 or 2. In some embodiments, z is 1, In some embodiments, z is 2. In some embodiments of Formula V, R⁴ is H and R⁵ is H or C₁₋₅alkyl. In some embodiments, the C₁₋₅alkyl is methyl, ethyl, propyl, isopropyl, butyl, t-butyl, s-butyl, or isoamyl. In some embodiments, the heteroaryl is a 5- or 6-membered heteroaryl having 1, 2, or 3 heteroatoms selected from the group consisting of N, O, and S. In some embodiments, an R⁴ and R⁵ taken together with the nitrogen atom to which they are attached form a C₃₋₆cycloalkyl.

In some embodiments of Formula V, R⁶ is alkyl, haloalkyl, alkoxy, —N(H)alkyl, or —N(H)aryl. In some embodiments, R⁶ is alkyl, haloalkyl, or alkoxy. In some embodiments, R⁶ is alkyl. In some embodiments, the alkyl is Me, Et, —CH₂O(C₁₋₅alkyl), —CH₂S(C₁₋₅alkyl), or —CH₂N(H)(C₁₋₅alkyl). In some embodiments, the alkyl is Me. In some embodiments, R⁶ is C₁₋₅alkyl, C₁₋₅alkoxy, or —N(H)C₁₋₅alkyl. In some embodiments, R⁶ is a C₁₋₅alkyl. In some embodiments, the C₁₋₅alkyl is methyl, ethyl, propyl, isopropyl, butyl, t-butyl, s-butyl, or isoamyl. In some embodiments, the C₁₋₅alkyl is methyl. In some embodiments, the C₁₋₅alkoxy is —OMe, —OEt, —OPr, —OiPr, —OBu, or —OtBu. In some embodiments, R⁶ is Me, CH₂CF₃, or —OMe. In some embodiments, R⁶ is a C₂₋₅alkyl.

In some embodiments of Formula V, R⁷ and R⁸ are each independently H, C₁₋₅alkyl, —CH₂aryl, or —CH₂heteroaryl. In some embodiments, R⁷ and R⁸ are each independently H or C₁₋₅alkyl. In some embodiments, the C₁₋₅alkyl is methyl, ethyl, propyl, isopropyl, butyl, t-butyl, s-butyl, or isoamyl. In some embodiments, the C₁₋₅alkyl is methyl. In some embodiments, the heteroaryl is a 5- or 6-membered heteroaryl having 1, 2, or 3 heteroatoms selected from the group consisting of N, O, and S. In some embodiments, the heteroaryl is pyridyl, pyrimidyl, oxazolyl, thiazolyl, imidazolyl, pyrazolyl, triazolyl, thiophenyl, or quinolyl. In some embodiments, the aryl is a phenyl.

In some embodiments of Formula V, R⁹ is H, halogen, C₁₋₅alkyl, C₁₋₂ haloalkyl, or C₁₋₅alkoxy. In some embodiments, the C₁₋₅alkyl is Me, Et, Pr, or iPr. In some embodiments, the C₁₋₅alkoxy is —OMe, —OEt, or —OiPr. In some embodiments, the C₁₋₂ haloalkyl is CF₃, CHF₂, or CH₂F. In some embodiments, the halogen in F or Cl. In some embodiments, R⁹ is methyl, F, CF₃, or —OMe.

In some embodiments of Formula V, m is 1, 2, or 3. In some embodiments, m is 1 or 2. In some embodiments, m is 1. In some embodiments, m is 2.

In some embodiments of Formula V, n is 1. In some embodiments, n is 2.

In some embodiments of Formula V, when n is 1, R^{a} is not H. In some embodiments, when n is 1, R^{a} is alkyl, haloalkyl, aryl, alkylene-cycloalkyl, alkylene-heterocycloalkyl, heteroaryl, alkylene-aryl, or alkylene-heteroaryl. In some embodiments, when n is 1, R^{a} is C₁₋₅alkyl, e.g., Me, Et, Pr, or iPr. In some embodiments of Formula V, when n is 2, one R^{a} is H.

In some embodiments, the compound of the present disclosure is not one or more compounds selected from the group consisting of:

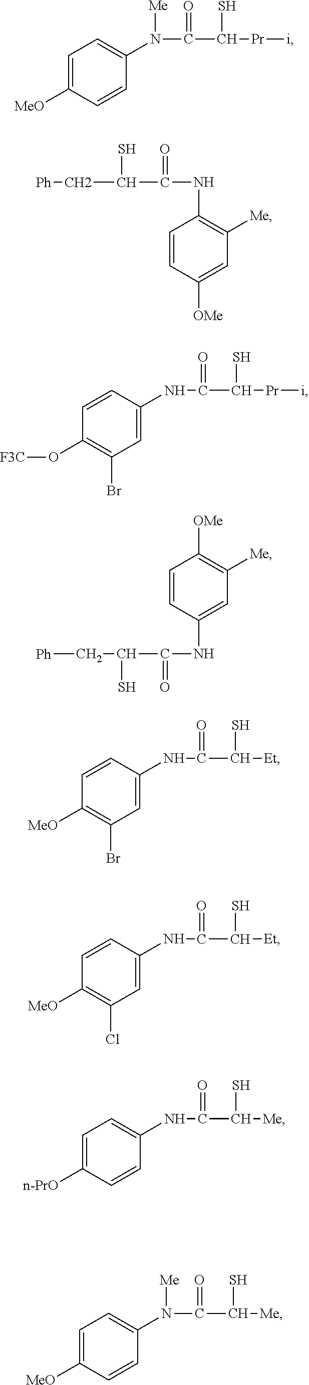

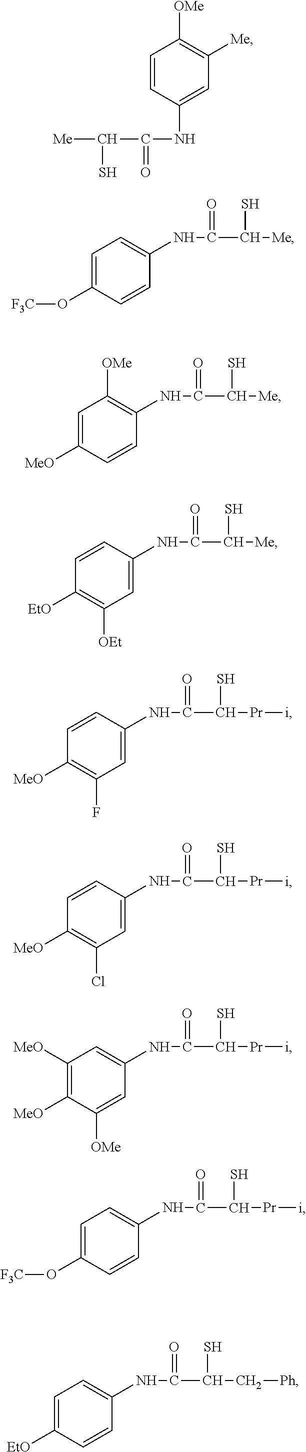

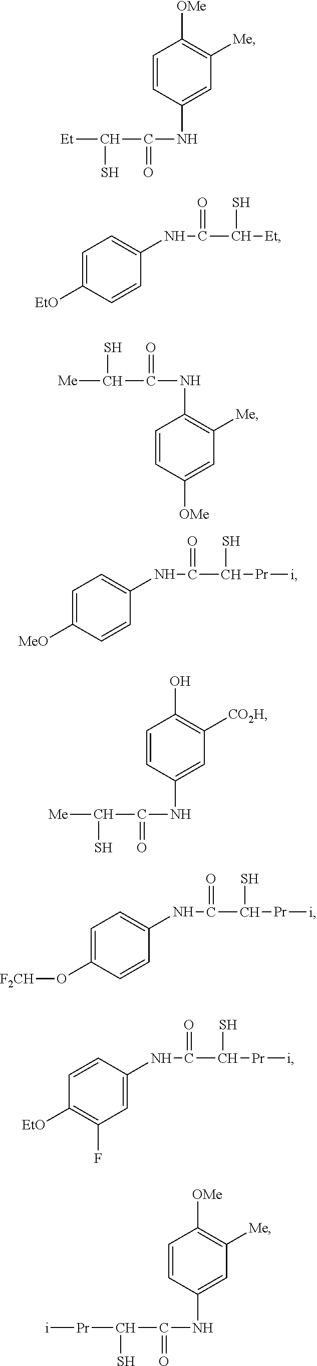

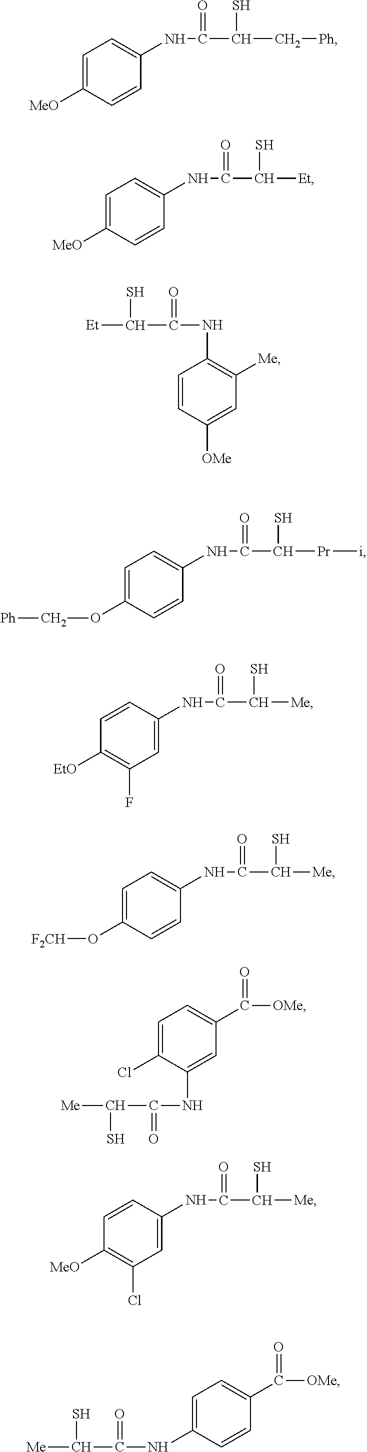

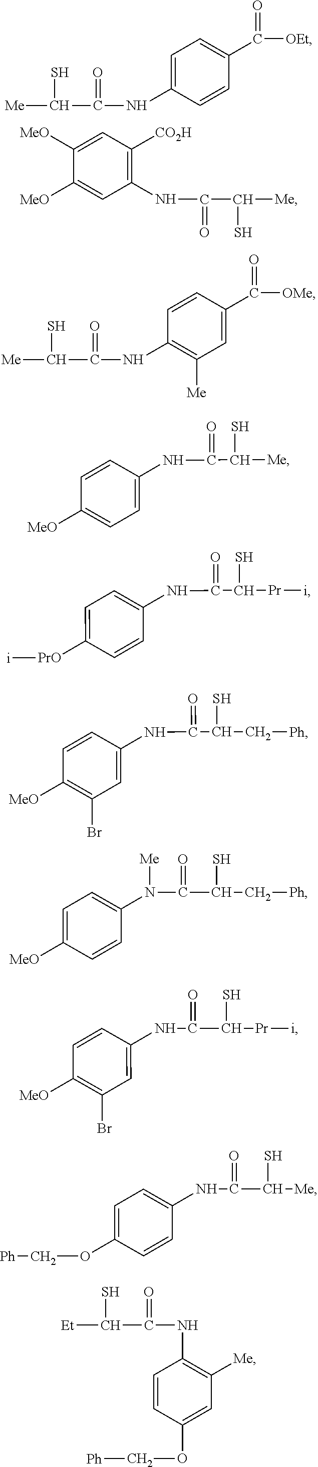

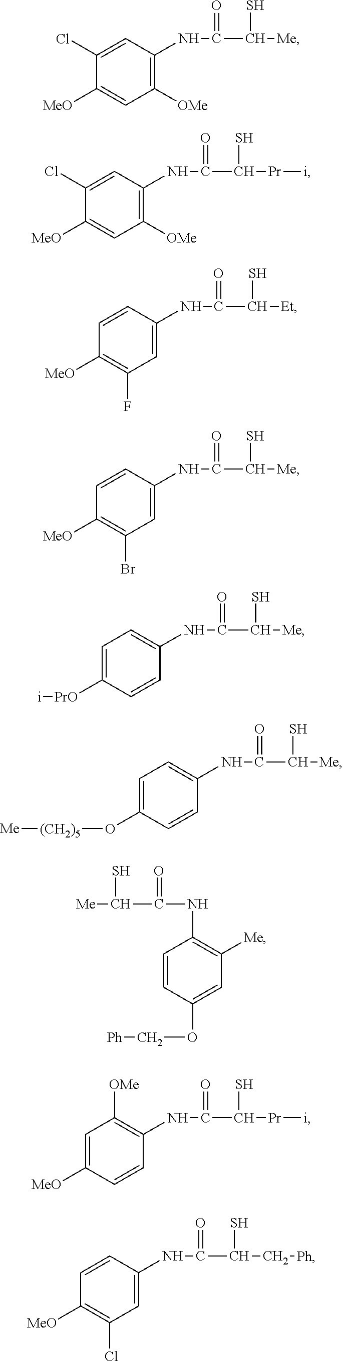

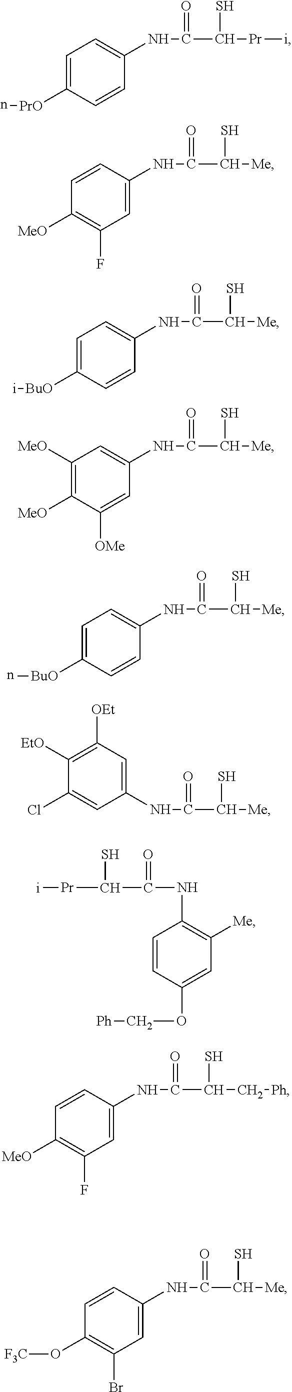

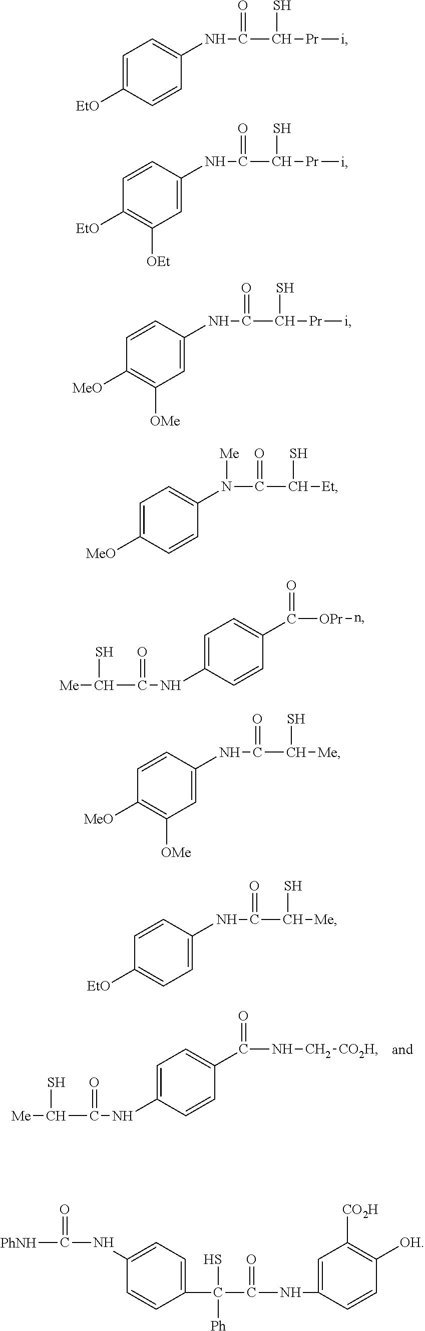

In some embodiments, the compound of the present disclosure is not:

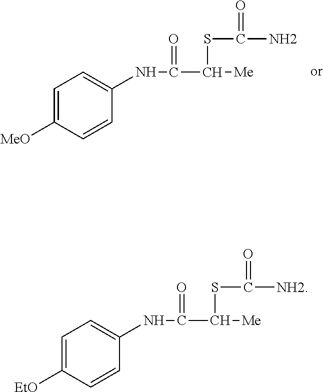 [SMILES: COc1ccc(NC(=O)C(C)SC(N)=O)cc1 ; CCOc1ccc(NC(=O)C(C)SC(N)=O)cc1]

In some embodiments, the compound of the present disclosure is not:

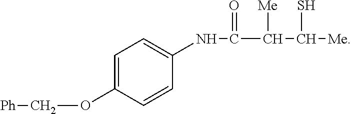 [SMILES: CC(S)C(C)C(=O)Nc1ccc(OCc2ccccc2)cc1]

In some embodiments, the compound of the present disclosure is not:

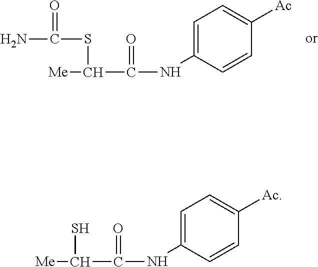 [SMILES: CC(=O)c1ccc(NC(=O)C(C)SC(N)=O)cc1 ; CC(=O)c1ccc(NC(=O)C(C)S)cc1]

In some embodiments, the present disclosure provides a compound of Formula VA:

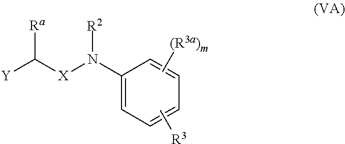

or a stereoisomer or a pharmaceutically acceptable salt thereof, wherein X, Y, R^{a}, R², R³, R^{3a}, and m are as defined herein, e.g., in Formula V.

In some embodiments, the present disclosure provides a compound of Formula VB:

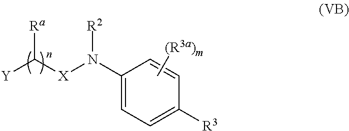

or a stereoisomer or a pharmaceutically acceptable salt thereof, wherein X, Y, R^{a}, R², R³, R^{3a}, m and n are as defined herein, e.g., in Formula V.

In some embodiments, the compound of Formula V is a compound of Formula VB:

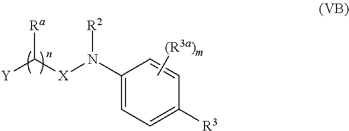

or a stereoisomer or a pharmaceutically acceptable salt thereof, wherein:
X is —C(O)— or —S(O)₂—;
Y is —SC(O)R¹ or —SH;
R^{a} is H, alkyl, haloalkyl, aryl, alkylene-cycloalkyl, alkylene-heterocycloalkyl, heteroaryl, alkylene-aryl, or alkylene-heteroaryl;
R¹ is alkyl or —NR⁴R⁵;
R² is H, alkyl, or cycloalkyl;
R³ is alkoxy, —O-alkylene-NR⁷R⁸, —C(O)R⁶, or —C(O)N(H)-alkylene-C(O)NR⁷R⁸,
wherein the alkylene is optionally substituted;
R^{3a} is alkyl, halogen, alkoxy, haloalkyl or haloalkoxy;
R⁴, R⁵, R⁷, and R⁸ are each independently H, alkyl, cycloalkyl, —CH₂cycloalkyl, aryl, or —CH₂aryl;
R⁶ is C₂₋₅alkyl, haloalkyl, or —N(H)alkyl;
m is 0, 1, 2, or 3; and
n is 1 or 2.

In some embodiments, the compound of Formula VB has the structure:

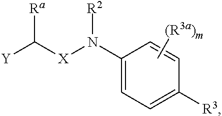

or a pharmaceutically acceptable salt thereof, wherein X, Y, R^{a}, R², R³, R^{3a}, and m are as defined herein, e.g., in Formula V.

In some embodiments, the compound of Formula VB has the structure:

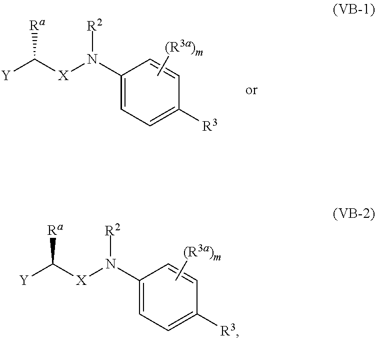

or a pharmaceutically acceptable salt thereof, wherein X, Y, R^{a}, R², R³, R^{3a}, and m are as defined herein, e.g., in Formula V.

In some embodiments, the present disclosure provides a compound of Formula VC:

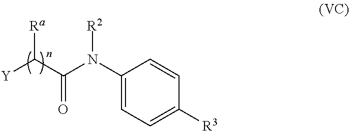

or a stereoisomer or a pharmaceutically acceptable salt thereof, wherein Y, R^{a}, R², R³, and n are as defined herein, e.g., in Formula V.

In some embodiments, the present disclosure provides a compound of Formula VD:

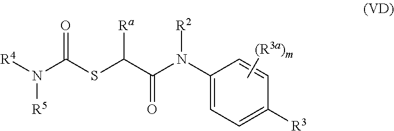

or a stereoisomer or a pharmaceutically acceptable salt thereof, wherein R^{a}, R², R³, R⁴, R⁵, R^{3a} and m are as defined herein, e.g., in Formula V.

In some embodiments, the compound of Formula VD has the structure:

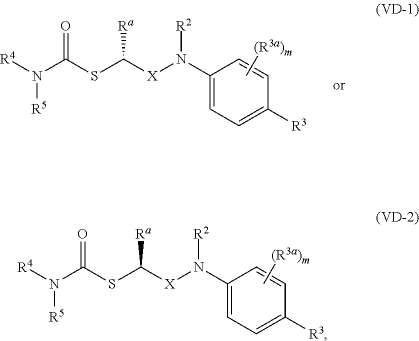

or a pharmaceutically acceptable salt thereof, wherein R^{a}, R², R³, R⁴, R⁵, R^{3a} and m are as defined herein, e.g., in Formula V.

In some embodiments, the compound of the present disclosure is selected from:

| Cmpd. | |
|---|---|
| # | Structure |
| 1 | 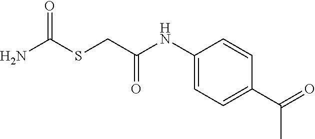 [SMILES: CC(=O)c1ccc(NC(=O)CSC(N)=O)cc1] |
| 2 | 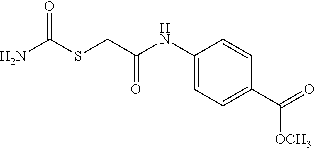 [SMILES: COC(=O)c1ccc(NC(=O)CSC(N)=O)cc1] |
| 3 | 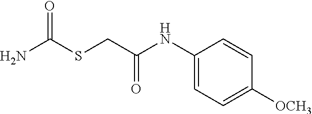 [SMILES: COc1ccc(NC(=O)CSC(N)=O)cc1] |
| 4 | 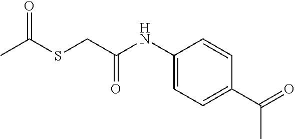 [SMILES: CC(=O)SCC(=O)Nc1ccc(C(C)=O)cc1] |
| 5 | 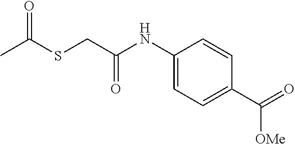 [SMILES: COC(=O)c1ccc(NC(=O)CSC(C)=O)cc1] |
| 6 | 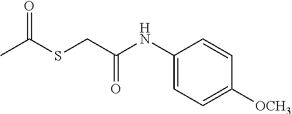 [SMILES: COc1ccc(NC(=O)CSC(C)=O)cc1] |
| 7 | 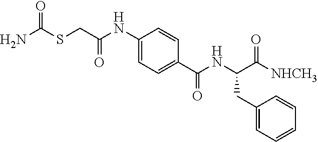 [SMILES: CNC(=O)[C@H](Cc1ccccc1)NC(=O)c1ccc(NC(=O)CSC(N)=O)cc1] |
| 8 | 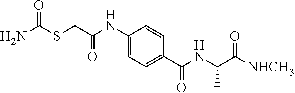 [SMILES: CNC(=O)[C@H](C)NC(=O)c1ccc(NC(=O)CSC(N)=O)cc1] |
| 9 | 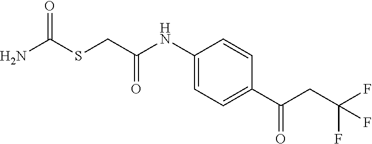 [SMILES: NC(=O)SCC(=O)Nc1ccc(C(=O)CC(F)(F)F)cc1] |
| 10 | 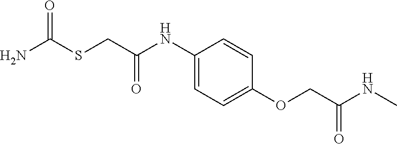 [SMILES: CNC(=O)COc1ccc(NC(=O)CSC(N)=O)cc1] |
| 11 | 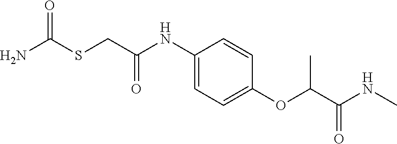 [SMILES: CNC(=O)C(C)Oc1ccc(NC(=O)CSC(N)=O)cc1] |
| 12 | 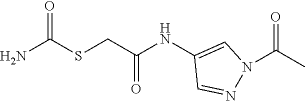 [SMILES: CC(=O)n1cc(NC(=O)CSC(N)=O)cn1] |
| 13 | 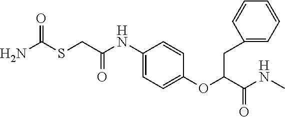 [SMILES: CNC(=O)C(Cc1ccccc1)Oc1ccc(NC(=O)CSC(N)=O)cc1] |
| 14 | 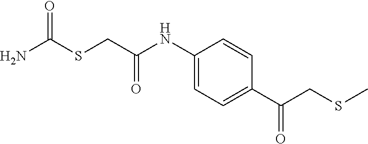 [SMILES: CSCC(=O)c1ccc(NC(=O)CSC(N)=O)cc1] |
| 15 | 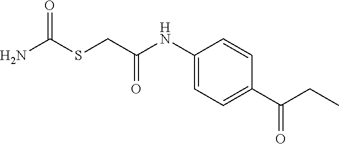 [SMILES: CCC(=O)c1ccc(NC(=O)CSC(N)=O)cc1] |
| 17 | 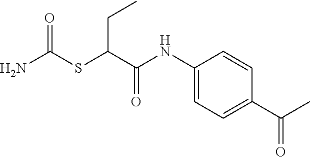 [SMILES: CCC(SC(N)=O)C(=O)Nc1ccc(C(C)=O)cc1] |
| 18 | 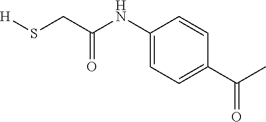 |
| 19 | 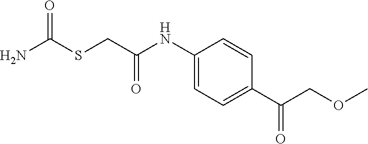 [SMILES: COCC(=O)c1ccc(NC(=O)CSC(N)=O)cc1] |
| 20 | 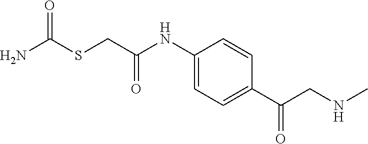 [SMILES: CNCC(=O)c1ccc(NC(=O)CSC(N)=O)cc1] |
| 21 | 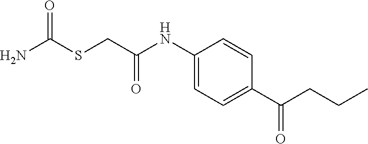 [SMILES: CCCC(=O)c1ccc(NC(=O)CSC(N)=O)cc1] |
| 22 | 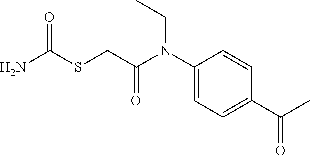 [SMILES: CCN(C(=O)CSC(N)=O)c1ccc(C(C)=O)cc1] |
| 24 | (−)-17 |
| | Sp.Opt. Rot. = −178.12^{b} |
| 25 | (+)-17 |
| | Sp.Opt. Rot. = +189.76^{b} |
| 26 | 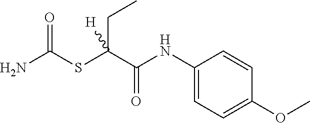 [SMILES: CCC(SC(N)=O)C(=O)Nc1ccc(OC)cc1] |
| 27 | 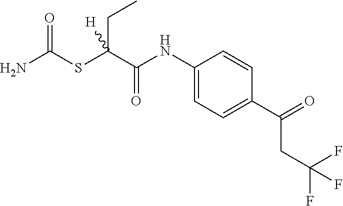 [SMILES: CCC(SC(N)=O)C(=O)Nc1ccc(C(=O)CC(F)(F)F)cc1] |
| 28 | 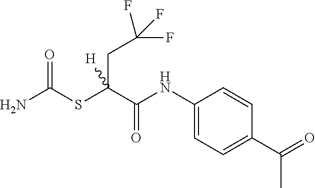 [SMILES: CC(=O)c1ccc(NC(=O)C(CC(F)(F)F)SC(N)=O)cc1] |
| 29 | 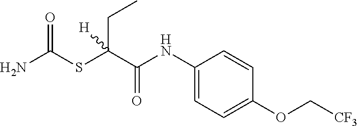 [SMILES: CCC(SC(N)=O)C(=O)Nc1ccc(OCC(F)(F)F)cc1] |
| 30 | 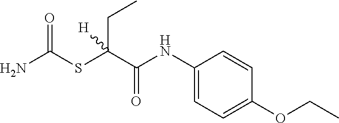 [SMILES: CCOc1ccc(NC(=O)C(CC)SC(N)=O)cc1] |
| 31 | 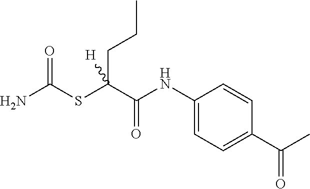 [SMILES: CCCC(SC(N)=O)C(=O)Nc1ccc(C(C)=O)cc1] |
| 34 | 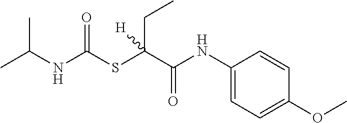 [SMILES: CCC(SC(=O)NC(C)C)C(=O)Nc1ccc(OC)cc1] |
| 35 | 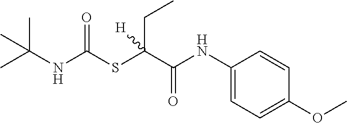 [SMILES: CCC(SC(=O)NC(C)(C)C)C(=O)Nc1ccc(OC)cc1] |
| 36 | 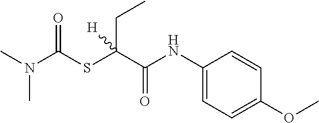 |
| 37 | 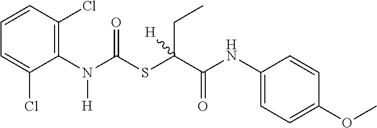 [SMILES: CCC(SC(=O)Nc1c(Cl)cccc1Cl)C(=O)Nc1ccc(OC)cc1] |
| 38 | 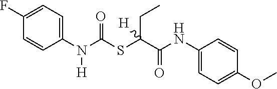 [SMILES: CCC(SC(=O)Nc1ccc(F)cc1)C(=O)Nc1ccc(OC)cc1] |
| 39 | 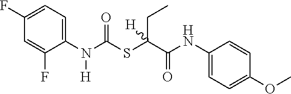 [SMILES: CCC(SC(=O)Nc1ccc(F)cc1F)C(=O)Nc1ccc(OC)cc1] |
| 40 | 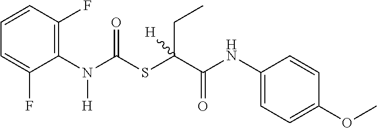 [SMILES: CCC(SC(=O)Nc1c(F)cccc1F)C(=O)Nc1ccc(OC)cc1] |

In some embodiments, the compound of the present disclosure (e.g., a compound of Formula V, VA, VB, VC, or VD) is selected from the group consisting of:

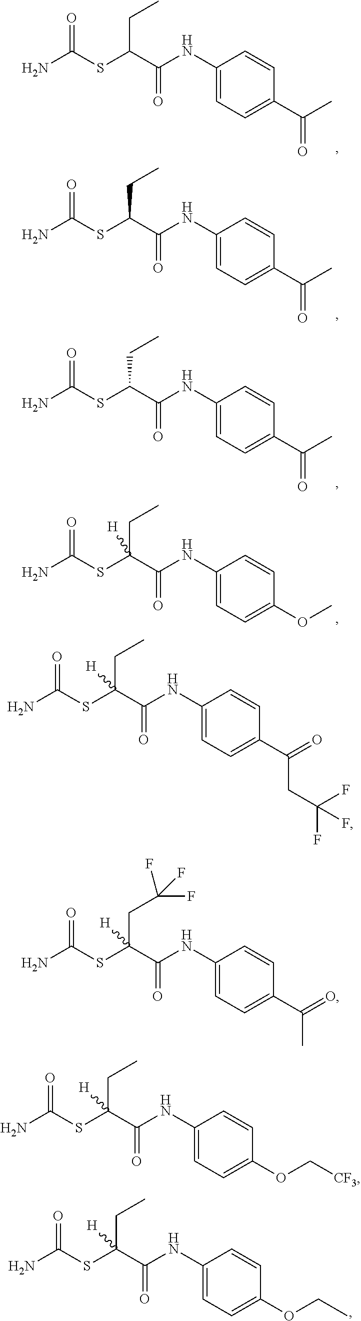

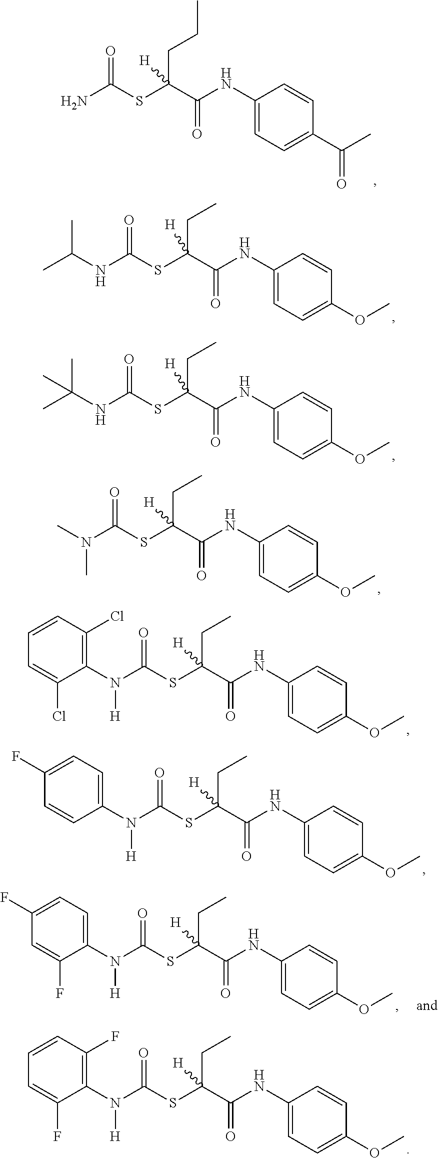

In some embodiments, the compound of the present disclosure is a compound in Table 8. In some embodiments, the compound of the present disclosure is a stereoisomer or a pharmaceutically acceptable salt of any of the compounds listed in Table 8.

Pharmaceutically acceptable derivatives of a compound may include salts, esters, enol ethers, enol esters, acetals, ketals, orthoesters, hemiacetals, hemiketals, acids, bases, solvates, hydrates or prodrugs thereof. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, aralkyl and cycloalkyl. Pharmaceutically acceptable enol esters include, but are not limited to, derivatives of formula C═C(OC(O)R) where R is hydrogen, alkyl, alkenyl, alkynyl, aryl, aralkyl and cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

A compound of the present disclosure (e.g., a compound of Formula I, Formula II, Formula III, Formula IV, Formula V, Formula VA, Formula VB, Formula VC, or Formula VD) may contain a chiral center. Such chiral center may be either of the (R) or (S) configuration, or may be a mixture thereof. The compound may be enantiomerically pure, or may be stereoisomeric or diastereomeric mixtures. In embodiments wherein the compound undergoes epimerization in vivo, administration of a compound in its (R) form is equivalent to administration of the compound in its (S) form.

In some embodiments, the compound of the present disclosure (e.g., a compound of Formula I, Formula II, Formula III, Formula IV, Formula V, Formula VA, Formula VB, Formula VC, or Formula VD) has an inhibition constant (Ki) of less than about 100 mM, less than about 10 mM, less than about 1 mM, less than about 0.1 mM, less than about 0.01 mM, less than about 0.001 mM, less than about 0.0001 mM, or less than about 0.00001 mM. In some embodiments, the compound has an inhibition constant in the range of about 10⁻⁵ to about 10⁻¹³ M, such as about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³ M. The term “inhibition constant” denotes the concentration of inhibitor required to produce half maximum inhibition of an enzyme.

In some embodiments, the compound of the present disclosure has an IC50 of less than about 100 mM, less than about 10 mM, less than about 1 mM, less than about 0.1 mM, less than about 0.01 mM, less than about 0.001 mM, less than about 0.0001 mM, or less than about 0.00001 mM. In some embodiments, a compound has an IC50 in the range of about 1 μM to about 500 μM. In some embodiments, a compound has an IC50 in the range of about 0.1 to about 10 nm, about 10 nm to about 100 inm, about 100 nm to about 500 nm, about 500 nm to about 1 μM, about 1 μM to about 10 μM, about 10 μM to about 100 μM, about 100 μM to about 500 μM, about 500 μM to about 1 mM, or about 1 mM to about 100 mM. As used herein, IC50 is the half maximal inhibitor concentration (i.e., a measure of the potency of a substance in inhibiting a specific biological or biochemical function). IC50 may be determined using standard inhibition assays known in the art. For example in some embodiments, the IC50 of a small molecule inhibitor may be determined by measuring cleavage of a FRET-based peptide substrate. The FRET-based peptide substrate may be, for example, Anaspec AS-27077, which has the sequence Mca-Pro-Leu-Gly-Leu-Dap(Dnp)-Ala-Arg-NH₂ (SEQ ID NO: 10), wherein Mca stands for 7-methoxy-coumarin-4-yl acetic acid-2,4-dinitrophenyl-lysine, and Dap(Dnp) stands for N^{β}-2,4-dinitrophenyl-L-di-aminopropionic acid.

### Pharmaceutical Compositions

Also provided herein are pharmaceutical compositions comprising one or more compounds of the present disclosure. In some embodiments, a pharmaceutical composition comprises one or more compounds disclosed herein (e.g., a compound of Formula I, Formula II, Formula IIA, Formula III, Formula IV, Formula V, Formula VA, Formula VB, Formula VC, or Formula VD) and one or more pharmaceutically acceptable carriers or excipients. A non-limiting list of pharmaceutically acceptable carriers and excipients is disclosed in Adejare, A. (Ed.). (2020) Remington: The Science and Practice of Pharmacy, 23^{rd} Edition. Elsevier, which is hereby incorporated by reference in its entirety for all purposes.

A pharmaceutical composition can be prepared using conventional pharmaceutically acceptable excipients and additives and conventional techniques. Such pharmaceutically acceptable excipients and additives include, but are not limited to, non-toxic compatible fillers, binders, disintegrants, buffers, preservatives, anti-oxidants, lubricants, flavorings, thickeners, coloring agents, emulsifiers and the like.

In some embodiments, the concentration of the inhibitor in the pharmaceutical composition range from about 1 nanomolar to about 1 micromolar, from about 1 micromolar to about 1 millimolar, of from about 1 millimolar to about 1 molar. In some embodiments, the concentration of the inhibitor is about 10 micromolar, about 25 micromolar, about 50 micromolar, about 75 micromolar, about 100 micromolar, about 250 micromolar, or about 500 micromolar.

The pharmaceutical composition can be formulated for administration systemically or locally. In some embodiments, the pharmaceutical composition is formulated for administration orally, parenterally, sublingually, transdermally, rectally, transmucosally, topically, via inhalation, via buccal administration, intrapleurally, intravenously, intraarterially, intragastrically, nasally, intraperitoneally, subcutaneously, intramuscularly, intranasally, intrathecally, and intraarticularly or combinations thereof. In some embodiments, the pharmaceutical composition can be formulated for oral administration. In some embodiments, the pharmaceutical composition can be formulated for intravenous administration.

For oral administration, the pharmaceutical compositions can take the form of, for example, tablets, capsules, or lozenges, prepared by conventional means with pharmaceutically acceptable excipients. In some embodiments, the pharmaceutical composition is formulated as a liquid. Liquid preparations can take the form of, for example, elixirs, solutions, syrups or suspensions, or they can be presented as dry product for constitution with water or other suitable vehicle before use. Oral administration also includes enteric formulations, which may include acid stable agents that maintain activity under gastrointestinal conditions, enteric coatings of pills, and the like, where there is a significant activity of the agent in intestinal tissues.

Injectables can be prepared in conventional forms, either as liquid solutions or suspensions, solid forms suitable for solution or suspension in liquid prior to injection, or as emulsions. The injectables, solutions and emulsions can also contain one or more excipients. Excipients include, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

In some embodiments, the pharmaceutical composition is formulated for intranasal administration. Numerous delivery devices are available for intranasal administration such as instillation catheters, droppers, unit-dose containers, squeeze bottles pump sprays, airless and preservative-free sprays, compressed air nebulizers, metered-dose inhalers, insufflators and pressurized metered dose inhalers. Devices vary in accuracy of delivery, dose reproducibility, cost, and ease of use. Currently, metered-dose systems provide the greatest dose accuracy and reproducibility.

### Methods of Treatment

The present disclosure relates to methods of treating or preventing a disease or disorder in a subject, the method comprising administering a therapeutically effective amount of a compound of the present disclosure (e.g., a compound of Formula I, Formula II, Formula III, Formula IV, Formula V, Formula VA, Formula VB, Formula VC, or Formula VD) or composition thereof to the subject in need thereof.

In some embodiments, the present disclosure provides a method of treating an inflammatory bowel disease or disorder in a subject, the method comprising administering a therapeutically effective amount of a compound of the present disclosure (e.g., a compound of Formula I, Formula II, Formula IIA, Formula III, Formula IV, Formula V, Formula VA, Formula VB, Formula VC, or Formula VD) or composition thereof to the subject in need thereof. In some embodiments, the inflammatory bowel disease or disorder is Crohn's disease or ulcerative colitis. In some embodiments, the methods of the disclosure may be used to treat ulcerative colitis, indeterminate colitis, microscopic colitis and collagenous colitis.

In some embodiments, the present disclosure provides a method of treating cancer in a subject, the method comprising administering a therapeutically effective amount of a compound of the present disclosure (e.g., a compound of Formula I, Formula II, Formula III, Formula IV, Formula V, Formula VA, Formula VB, Formula VC, or Formula VD) or composition thereof to the subject in need thereof. In some embodiments, the cancer is a gastrointestinal (GI) cancer. The GI cancer may be, for example, esophageal cancer, gallbladder cancer, liver cancer, pancreatic cancer, stomach cancer, cancer of the small intestine, colorectal cancer, and anal cancer. In some embodiments, the cancer is colorectal cancer, such as adenocarcinoma, gastrointestinal stromal tumors (GIST), colorectal lymphoma, carcinoids, Turcot Syndrome, Peutz-Jeghers Syndrome (PJS), Familial Colorectal Cancer (FCC), or Juvenile Polyposis Coli. The cancer may be stage I, stage II, stage Ill, or stage IV (i.e., metastatic).

In some embodiments, the present disclosure provides a method of treating a systemic bacterial infection in a subject in need thereof, the method comprising administering to the subject a therapeutically effective amount of a compound of the present disclosure (e.g., a compound of Formula I, Formula II, Formula IIA, Formula III, Formula IV, Formula V, Formula VA, Formula VB, Formula VC, or Formula VD) or composition thereof to the subject in need thereof. In some embodiments, the systemic bacterial infection is a systemic tissue infection. In some embodiments, the systemic bacterial infection is endocarditis or a urinary tract infection. In some embodiments, the systemic bacterial infection is septicemia.

In some embodiments of the disclosed methods, the subject is colonized by one or more pathogenic bacterial strain. Colonization may result in an acute infection, or result in a chronic infection. In some embodiments, the pathogenic bacterial strain is B. fragilis, E. faecalis, and/or C. perfringens. In some embodiments, the pathogenic bacterial strain is a strain of B. fragilis expressing the BFT toxin, a strain of E. faecalis expressing the gelatinase GelE, or a strain of C. perfringens expressing the collagenase ColA. In some embodiments, the pathogenic bacterial strain is a strain of B. fragilis expressing the BFT toxin. In some embodiments, the subject is colonized by B. fragilis, E. faecalis, or C. perfringens. In some embodiments, the subject is colonized by B. fragilis, E. faecalis, and C. perfringens. In some embodiments, the subject is colonized by B. fragilis and E. faecalis. In some embodiments, the subject is colonized by B. fragilis and C. perfringens. In some embodiments, the subject is colonized by E. faecalis and C. perfringens. In some embodiments, the subject is colonized by B. fragilis. In some embodiments, the subject is colonized by an enterotoxigenic strain of B. fragilis (ETBF). In some embodiments, a subject is colonized with more than one strain of ETBF. In some embodiments, a subject that is colonized with ETBF is also be colonized with one or more strains of NTBF. In some embodiments, colonization is by one or more strain of ETBF. In some embodiments, the subject is colonized by E. faecalis. In some embodiments, the subject is colonized by C. perfringens.

In some embodiments, the method for treating or preventing a disease or disorder in a subject comprises administering to the subject a therapeutically effective amount of a compound of the present disclosure (e.g., a compound of Formula I, Formula II, Formula IIA, Formula III, or Formula IV) that diminishes the pathogenic effects of a strain of B. fragilis expressing the BFT toxin, a strain of E. faecalis expressing the gelatinase GelE, or a strain of C. perfringens expressing the collagenase ColA.

In some embodiments, the method for treating or preventing a disease or disorder in a subject comprises administering to the subject a compound that binds to and/or inhibits the activity of one or more of BFT, ColA, and GelE. In some embodiments, the compound binds to BFT, ColA, and/or GelE with an inhibition constant in the range of about 10⁻⁵ to about 10⁻¹³ M, e.g., about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³ M. In some embodiments, the method for treating or preventing a disease or disorder in a subject comprises administering to the subject an inhibitor of BFT, ColA, and/or GelE or a pharmaceutical composition thereof. In some embodiments, the BFT comprises the amino acid sequence of any one of SEQ ID NO: 2-4. In some embodiments, the BFT comprises an amino acid sequence that is at least 95%, at least 96%, at least 97%, or at least 98% identical to any one of SEQ ID NO: 2-4. In some embodiments, the BFT comprises an amino acid sequence that is at least 98% identical to any one of SEQ ID NO: 2-4. In some embodiments, the GelE comprises the amino acid sequence of SEQ ID NO: 6. In some embodiments, the GelE comprises an amino acid sequence that is at least 95%, at least 96%, at least 97%, or at least 98% identical to SEQ ID NO: 6. In some embodiments, the GelE comprises an amino acid sequence that is at least 98% identical to SEQ ID NO: 6. In some embodiments, the ColA comprises the amino acid sequence of SEQ ID NO: 8. In some embodiments, the ColA comprises an amino acid sequence that is at least 95%, at least 96%, at least 97%, or at least 98% identical to SEQ ID NO: 8. In some embodiments, the ColA comprises an amino acid sequence that is at least 98% identical to SEQ ID NO: 8.

In some embodiments of the disclosed methods, administering the compound reduces and/or eliminates the activity of at least one of BFT, ColA and/or GelE. In some embodiments of the disclosed methods, administering the compound reduces the activity of at least one of BFT, ColA and/or GelE. In some embodiments of the disclosed methods, administering the compound eliminates the activity of at least one of BFT, ColA and/or GelE. In some embodiments, administering the compound substantially eliminates the activity of at least one of BFT, CoL and/or GelE. In some embodiments, administering the compound completely eliminates the activity of at least one of BFT, ColA and/or GelE. In some embodiments, administering the compound (e.g., an inhibitor of BFT, ColA, and/or GelE) reduces the number of pathogenic bacteria in the subject. In some embodiments, administering the compound eliminates the infection caused by the pathogenic bacteria in the subject. In some embodiments, the pathogenic bacteria is one or more of B. fragilis, E. faecalis, and C. perfringens.

In some embodiments, the disease or disorder is an inflammatory bowel disease or disorder, such as Crohn's disease or ulcerative colitis. In some embodiments, the disease or disorder is a diarrheal disease, such as short duration watery diarrhea (e.g., due to cholera), short duration bloody diarrhea (e.g., dysentery), and persistent diarrhea (e.g., lasting more than 14 days). In some embodiments, the disease is cancer. In some embodiments, the cancer is a gastrointestinal (GI) cancer. The GI cancer may be, for example, esophageal cancer, gallbladder cancer, liver cancer, pancreatic cancer, stomach cancer, cancer of the small intestine, colorectal cancer, and anal cancer. In some embodiments, the cancer is colorectal cancer, such as adenocarcinoma, gastrointestinal stromal tumors (GIST), colorectal lymphoma, carcinoids, Turcot Syndrome, Peutz-Jeghers Syndrome (PJS), Familial Colorectal Cancer (FCC), or Juvenile Polyposis Coli. The cancer may be stage I, stage II, stage III, or stage IV (i.e., metastatic).

In some embodiments, the subject has (or is suspected of having) one or more diseases or disorders. In some embodiments, the subject has (or is suspected of having) an inflammatory bowel disease or disorder, such as Crohn's disease or ulcerative colitis. In some embodiments, the subject has (or is suspected of having) a diarrheal disease, such as short duration watery diarrhea (e.g., due to cholera), short duration bloody diarrhea (e.g., dysentery), and persistent diarrhea (e.g., lasting more than 14 days). In some embodiments, the subject has a gastrointestinal (GI) cancer. The GI cancer may be, for example, esophageal cancer, gallbladder cancer, liver cancer, pancreatic cancer, stomach cancer, cancer of the small intestine, colorectal cancer, and anal cancer. In some embodiments, the subject has colorectal cancer, such as adenocarcinoma, gastrointestinal stromal tumors (GIST), colorectal lymphoma, carcinoids, Turcot Syndrome, Peutz-Jeghers Syndrome (PJS), Familial Colorectal Cancer (FCC), or Juvenile Polyposis Coli. The cancer may be stage I, stage II, stage Ill, or stage IV (i.e., metastatic).

In some embodiments, the subject is a mammal, such as a primate, ungulate (e.g., cow, pig, horse), domestic pet or domesticated mammal. In some cases, the subject is a mammal selected from a rabbit, pig, horse, sheep, cow, cat or dog. In some embodiments, the subject is a human. The subject may be a male, or a female. In some embodiments, the subject is greater than about 18 years old, greater than about 25 years old, greater than about 35 years old, greater than about 45 years old, greater than about 55 years old, greater than about 65 years old, greater than about 75 years old, or greater than about 85 years old. In some embodiments, the subject is less than about 18 years old, less than about 16 years old, less than about 14 years old, less than about 12 years old, less than about 10 years old, less than about 8 years old, less than about 6 years old, less than about 5 years old, less than about 4 years old, less than about 3 years old, less than about 2 years old, less than about 1 year old, or less than about 6 months old. In some embodiments, the subject is greater than or equal to 18 years old. In some embodiments, the subject is less than 18 years old.

In some embodiments of the disclosed methods, the compound or pharmaceutical composition is administered to the subject orally, parenterally, sublingually, transdermally, rectally, transmucosally, topically, via inhalation, via buccal administration, intrapleurally, intravenously, intraarterially, intragastrically, nasally, intraperitoneally, subcutaneously, intramuscularly, intranasally, intrathecally, and intraarticularly or combinations thereof. In some embodiments, the compound is administered orally to the subject. In some embodiments, the compound is administered in a tablet or a capsule. In some embodiments, the tablet or capsule comprises a pharmaceutically acceptable carrier or excipient. In some embodiments, the compound is administered as a liquid formulation. In some embodiments, the liquid formulation comprises a pharmaceutically acceptable carrier or excipient. In some embodiments, the compound is administered intravenously to the subject.

The pharmaceutical compositions described herein may be administered at a therapeutically-effective dose. As used herein, “therapeutically-effective dose” means a dose sufficient to achieve the intended therapeutic purpose, such as, to alleviate a sign or symptom of a disease or disorder in a patient. A therapeutically effective amount of compound in this invention will vary with the particular goal to be achieved, the age and physical condition of the patient being treated, the severity of the underlying disease, the duration of treatment, the nature of concurrent therapy and the specific compound employed. For example, a therapeutically effective amount of a compound of the invention administered to a child or a neonate will be reduced proportionately in accordance with sound medical judgement. The effective amount of a compound of the invention will thus be the minimum amount which will provide the desired effect.

The amount of compound administered will depend upon a variety of factors, including, for example, the particular indication being treated, the route of administration, whether the desired benefit is prophylactic or therapeutic, the severity of the indication being treated and the age and weight of the patient, the bioavailability of the particular active compound, and the like. Determination of an effective dosage is well within the capabilities of those skilled in the art.

Effective dosages can be estimated initially from in vitro assays. For example, an initial dosage for use in animals can be formulated to achieve a circulating blood or serum concentration of active compound that is at or above an IC50 of the particular compound as measured in an in vitro assay. Calculating dosages to achieve such circulating blood, serum, or intestinal concentrations taking into account the bioavailability of the particular compound is well within the capabilities of skilled artisans. For guidance, see Fingl & Woodbury, “General Principles,” In: Goodman and Gilman's The Pharmaceutical Basis of Therapeutics, Chapter 1, pp. 1-46, latest edition, Pagamonon Press, and the references cited therein, which are incorporated herein by reference.

Initial dosages also can be estimated from in vivo data, such as animal models. Animal models useful for testing the efficacy of compounds to treat or prevent the various diseases described above are well-known in the art.

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration, and various factors discussed above. In some embodiments, a dose of the compound administered to the subject is from about 0.001 to about 1000 mg/kg of body weight per day, e.g., about 0.001 mg/kg of body weight per day, about 0.01 mg/kg of body weight per day, about 0.1 mg/kg of body weight per day, about 1 mg/kg of body weight per day, about 10 mg/kg of body weight per day, about 100 mg/kg of body weight per day, or about 1000 mg/kg of body weight today, including all ranges and values therebetween. Dosage amount and interval can be adjusted individually to provide plasma levels of the compound(s) which are sufficient to maintain therapeutic or prophylactic effect. In cases of local administration or selective uptake, such as local topical administration, the effective local concentration of active compound(s) cannot be related to plasma concentration. Skilled artisans will be able to optimize effective local dosages without undue experimentation.

The inhibitor (or a pharmaceutical composition comprising the same) can be administered once per day, once per week, or multiple times per day (e.g., bid, tid, qid, etc.) or week. Administration frequency may depend upon, among other things, the indication being treated and the judgment of the prescribing physician. A treatment of a subject with a therapeutically effective amount of a compound can include a single treatment or, preferably, can include a series of treatments. In another example, a subject may be treated daily for several years in the setting of a chronic condition or illness. It will also be appreciated that the effective dosage used for treatment may increase or decrease over the course of a particular treatment.

The disclosures of each and every patent, patent application, and publication cited herein are hereby incorporated herein by reference in their entirety. While this disclosure contains references to specific embodiments, it is apparent that other embodiments and variations of this disclosure may be devised by others skilled in the art without departing from the true spirit and scope of the invention. The appended claims are intended to be construed to include all such embodiments and equivalent variations.

### NUMBERED EMBODIMENTS

1. A compound of Formula I:

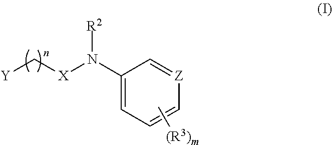

or a stereoisomer or a pharmaceutically acceptable salt thereof, wherein:
X is —C(O)— or —S(O)₂—;
Y is —SC(O)R¹ or —SH;
Z is CH or N;
R¹ is alkyl, haloalkyl, or —NR⁴R⁵;
R² is H, alkyl, or cycloalkyl;
R³ is —OH, alkoxy, —C(O)R⁶, or —C(O)N(H)-alkylene-C(O)N⁷R⁸, wherein the alkylene is optionally substituted;
R⁴, R⁵, R⁷, and R⁸ are each independently H, alkyl, cycloalkyl, —CH₂cycloalkyl, aryl, or —CH₂aryl;
R⁶ is alkyl, alkoxy, or —N(H)alkyl;
m is 0, 1, 2, or 3; and
n is 1 or 2.

2. The compound of embodiment 1, wherein X is —C(O)—.

3. The compound of embodiment 1 or 2, wherein R¹ is C₁₋₅alkyl or —NR⁴R⁵.

4. The compound of embodiment 3, wherein the C₁₋₅alkyl is methyl, ethyl, or isopropyl.

5. The compound of embodiment 3 or 4, wherein the C₁₋₅alkyl is methyl.

6. The compound of embodiment 4 or 5, wherein R⁴ and R⁵ are each H.

7. The compound of embodiment 4 or 5, wherein R⁴ is H and R⁵ is methyl.

8. The compound of any one of embodiments 1-7, wherein R² is H.

9. The compound of any one of embodiments 1-8, wherein R³ is alkoxy, —C(O)R⁶, or —C(O)N(H)-alkylene-C(O)N⁷R⁸.

10. The compound of any one of embodiments 1-8, wherein R³ is alkoxy or —C(O)R⁶.

11. The compound of any one of embodiments 1-10, wherein the alkoxy is —OMe.

12. The compound of any one of embodiments 1-11, wherein m is 1 or 2.

13. The compound of any one of embodiments 1-12, wherein m is 1.

14. The compound of any one of embodiments 1-13, wherein the R⁶ is Me or —OMe.

15. The compound of any one of embodiments 1-9, wherein the alkylene is methylene, optionally substituted with fluoro, alkyl, or —CH₂aryl.

16. The compound of any one of embodiments 1-9, wherein R⁷ and R⁸ are each independently H, Me, or —CH₂Ph.

17. The compound of any one of embodiments 1-9, wherein R⁷ is H and R⁸ is Me.

18. The compound of any one of embodiments 1-17, wherein Z is CH.

19. The compound of embodiment 1, wherein the compound of Formula (I) has a structure according to:

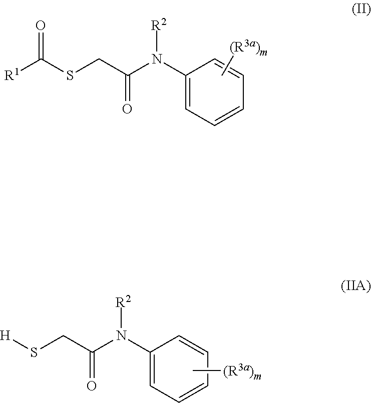

or a stereoisomer or a pharmaceutically acceptable salt thereof.

20. The compound of embodiment 19, wherein R¹ is C₁₋₅alkyl or —NR⁴R⁵.

21. The compound of embodiment 20, wherein the C₁₋₅alkyl is methyl.

22. The compound of embodiment 20 or 21, wherein R⁴ and R⁵ are each H.

23. The compound of any one of embodiments 19-22, wherein R² is H.

24. The compound of any one of embodiments 19-23, wherein R³ is alkoxy, —C(O)R⁶, or —C(O)N(H)-alkylene-C(O)N⁷R⁸.

25. The compound of any one of embodiments 19-24, wherein R³ is alkoxy or —C(O)R⁶.

26. The compound of any one of embodiments 19-25, wherein the alkoxy is —OMe.

27. The compound of any one of embodiments 19-26, wherein the R⁶ is Me or —OMe.

28. The compound of any one of embodiments 19-24, wherein the alkylene is optionally substituted methylene.

29. The compound of embodiment 28, wherein methylene is optionally substituted with fluoro, alkyl, or —CH₂aryl.

30. The compound of any one of embodiments 19-24, wherein R⁷ and R⁸ are each independently H, C₁₋₅alkyl, or —CH₂Ph.

31. The compound of embodiment 30, wherein the C₁₋₅alkyl is Me.

32. The compound of any one of embodiments 19-24, wherein R⁷ is H and R⁸ is Me.

33. The compound of embodiment 1, wherein the compound is:

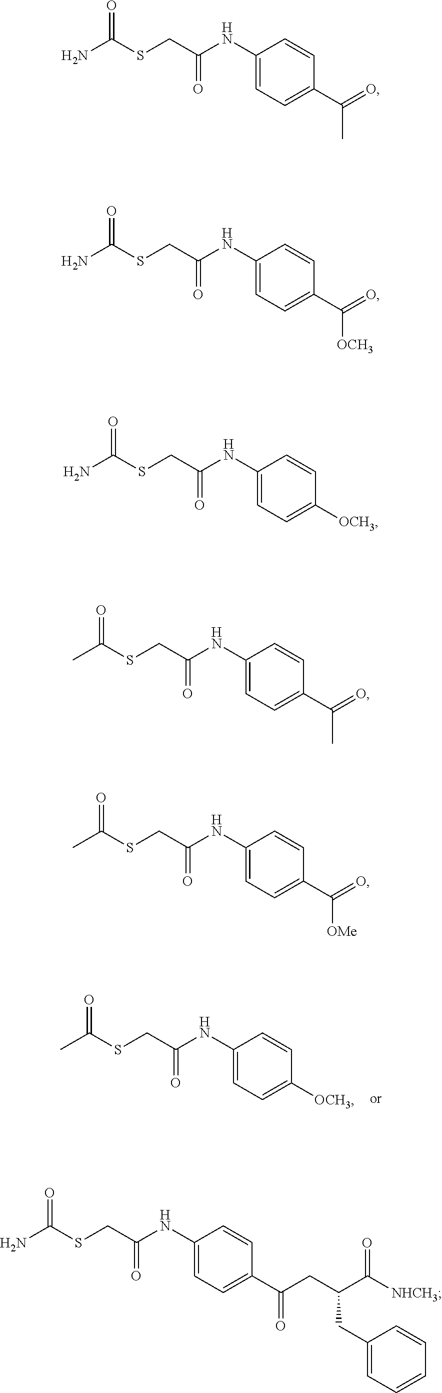

or a stereoisomer or a pharmaceutically acceptable salt thereof.

34. A compound of Formula VD:

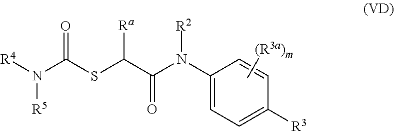

or a stereoisomer or a pharmaceutically acceptable salt thereof, wherein:
R^{a} is H, alkyl, haloalkyl, aryl, alkylene-cycloalkyl, alkylene-heterocycloalkyl, heteroaryl, alkylene-aryl, or alkylene-heteroaryl;
R² is H, alkyl, or cycloalkyl;
R³ is —OH, alkoxy, —C(O)R⁶, or —C(O)N(H)-alkylene-C(O)N⁷R⁸, wherein the alkylene is optionally substituted;
R⁴, R⁵, R⁷, and R⁸ are each independently H, alkyl, cycloalkyl, —CH₂cycloalkyl, aryl, or —CH₂aryl;
R⁶ is alkyl, alkoxy, or —N(H)alkyl;
R^{3a} is alkyl, halogen, alkoxy, haloalkyl or haloalkoxy; and
m is 0, 1, 2, or 3.

35. The compound of embodiment 34, wherein the compound is:

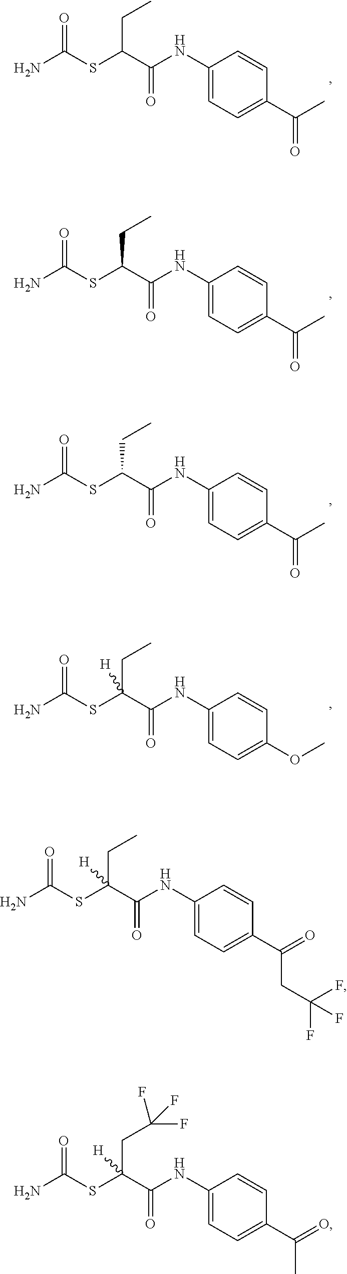 [SMILES: CCC(SC(N)=O)C(=O)Nc1ccc(C(C)=O)cc1 ; CC[C@H](SC(N)=O)C(=O)Nc1ccc(C(C)=O)cc1 ; CC[C@@H](SC(N)=O)C(=O)Nc1ccc(C(C)=O)cc1 ; CCC(SC(N)=O)C(=O)Nc1ccc(OC)cc1 ; CCC(SC(N)=O)C(=O)Nc1ccc(C(=O)CC(F)(F)F)cc1 ; CC(=O)c1ccc(NC(=O)C(CC(F)(F)F)SC(N)=O)cc1]

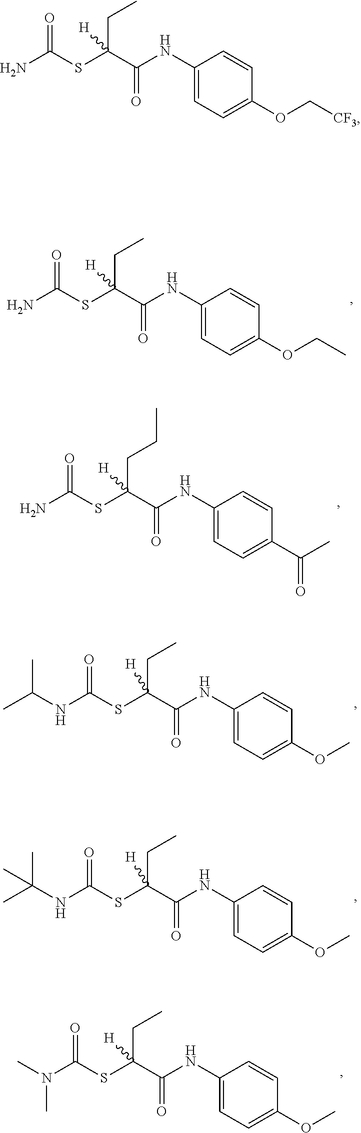

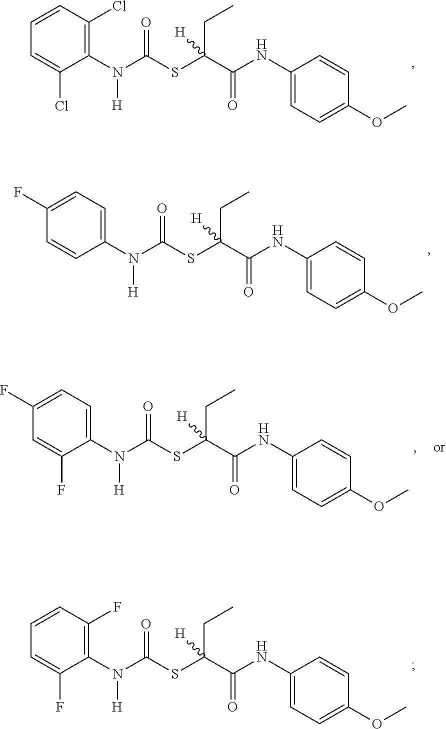 [SMILES: CCC(SC(=O)Nc1c(Cl)cccc1Cl)C(=O)Nc1ccc(OC)cc1 ; CCC(SC(=O)Nc1ccc(F)cc1)C(=O)Nc1ccc(OC)cc1 ; CCC(SC(=O)Nc1ccc(F)cc1F)C(=O)Nc1ccc(OC)cc1 ; CCC(SC(=O)Nc1c(F)cccc1F)C(=O)Nc1ccc(OC)cc1]

or a stereoisomer or a pharmaceutically acceptable salt thereof.

### EXAMPLES

The disclosure is further described in detail by reference to the following examples. These examples are provided for purposes of illustration only and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples therefore specifically point out the preferred embodiments and are not to be construed as limiting in any way the remainder of the disclosure.

TABLE 7
Abbreviations referred to herein

| Abbreviation | Description |
|---|---|
| mmol | Millimolar |
| vol | Volume |
| g | Grams |
| kg | Kilograms |
| L | Litres |
| mL | Millilitres |
| ° C. | Degrees celsius |
| TLC | Thin layer chromatography |
| HPLC | High-performance liquid chromatography |
| LCMS | Liquid chromatography - mass spectrometry |
| min | Minutes |
| h | Hours |
| eq | Equivalents |
| RT | Room temperature |
| Rf | Retention factor |
| RM | Reaction mixture |
| RP | Reversed phase |
| NMR | Nuclear magnetic resonance |
| ppm | Parts per million |

### Example 1

### Synthesis of S-(2-((4-acetylphenyl) amino)-2-oxoethyl) carbamothloate (1)

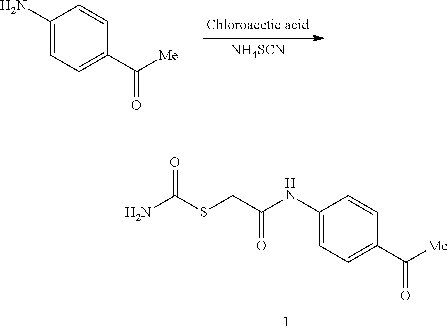 [SMILES: CC(=O)c1ccc(N)cc1 ; "NH4SCN" ; CC(=O)c1ccc(NC(=O)CSC(N)=O)cc1]

Synthesis of S-(2-((4-acetylphenyl) amino)-2-oxoethyl) carbamothloate (1): To a stirred solution of chloroacetic acid (349 mg, 3.699 mmol, 1 eq) and ammonium thiocyanate (281 mg, 3.699 mmol, 1 eq), was added in ethanol at room temperature. Reaction was stirred for 10 min, then 1-(4-aminophenyl) ethan-1-one (500 mg, 3.699 mmol, 1 eq), was added. Then reaction heated to 90° C. for 5 h and stirred at RT for 14 h. The reaction was monitored by TLC, after completion of the reaction, ice was added to reaction mixture and filtered to get brown solid. Crude was triturated with DCM (5 mL), and filtered to obtain S-(2-((4-acetylphenyl) amino)-2-oxoethyl) carbamothioate (310 mg, 33.2%) as an off white solid. TLC: 60% EtOAc/heptane (Rr: 0.4). ¹H NMR (DMSO-d₆, 400 MHz): δ 10.44 (s, 1H), 7.93 (br d, J=8.6 Hz, 2H), 7.71 (br d, J=8.7 Hz, 2H), 3.74 (s, 2H), 2.54-2.52 (m, 3H); LCMS: 93.34%, m/z=253.0 [M+H]⁺; (Column: EVO-C18 (3.0×50 mm, 2.6 μm); RT: 1.58 min, A: 0.025% Formic acid, B:ACN; T/B %: 0.01/5, 3/90, 5/90, 5.5/5, 6/5, 0.8 mL/min); HPLC: 90.66%; (Column; X-SELECT CSH C-18 (4.6×150 mm, 3.5 μm); RT: 5.44 min; A: 0.05% TFA:ACN (95:05), B:ACN: 0.05% TFA (95:05); T/B %: 0.01/10, 12/90, 16/90. Diluent: ACN:H₂O).

### Example 2

### Synthesis of (S)—S-(2-((4-((1-(methyl amino)-1-oxo-3-phenylpropan-2-yl) carbamoyl) phenyl) amino)-2-oxoethyl) carbamothloate (7)

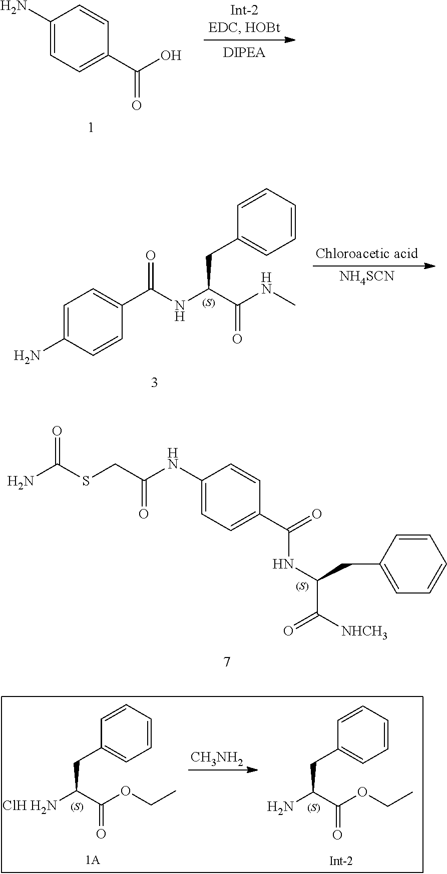

Synthesis of (S)-4-amino-N-(1-(methyl amino)-1-oxo-3-phenylpropan-2-yi) benzamide (3): To a stirred solution of 4-aminobenzoic acid (250 mg, 1.824 mmol, 1 eq) in DCM (3 mL) cooled at 0° C., were added EDC hydrochloride (271 mg, 2.736 mmol, 1.5 eq), hydroxybenzotriazole (369 mg, 2.736 mmol, 1.5 eq) and DIPEA (705 mg, 5.472 mmol, 3 eq), then reaction mixture stirred at 0° C. for 20 min. (S)-2-amino-N-methyl-3-phenylpropanamide (324 mg, 1.824 mmol, 1 eq) was added then allowed to warm to RT and stirred for 16 h. The reaction was monitored by TLC, after completion of the reaction added water and extracted with DCM (2×50 mL), the organic phases are dried over Na₂SO₄, filtered and concentrated under reduced pressure to get crude. The crude product purified by combiflash over silica gel column using 50% EtOAc/heptane as eluent to obtain (S)-4-amino-N-(1-(methyl amino)-1-oxo-3-phenylpropan-2-yl) benzamide (200 mg, 36.9%) as off white solid. TLC: 50% EtOAc/heptane (Rr: 0.3); LCMS: 94.05%, m/z=298.2 [M+H]⁺; ¹H NMR (DMSO-d₆, 400 MHz): δ 8.00 (d, J=8.3 Hz, 1H), 7.94-7.80 (m, 1H), 7.54 (d, J=8.6 Hz, 2H), 7.32-7.20 (m, 4H), 7.19-7.09 (m, 1H), 6.50 (d, J=8.6 Hz, 2H), 5.59 (s, 2H), 4.63-4.52 (m, 1H), 3.09-2.88 (m, 2H), 2.59 (d, J=4.6 Hz, 3H).

### Synthesis of (S)-2-amino-N-methyl-3-phenylpropanamide (Int-2)

To a stirred solution of methyl L-phenylalaninate hydrochloride (2.0 g, 8.70 mmol, 1 eq) in methanol (20 mL) was added 2M methylamine in methanol (6.53 mL, 13.06 mmol, 1.5 eq) at 0° C. and the reaction mixture was allowed to stir at RT for 16 h. The reaction was monitored by TLC, after completion of the reaction concentrated under vacuum. The residue was stirred in DCM (40 mL) and filtered. Combined filtrates were washed with water (5 mL) and brine solution (5 mL), separated the organic layer and dried over Na₂SO₄ and concentrated under vacuum to obtain (S)-2-amino-N-methyl-3-phenylpropanamide (1.45 g, 93.5%) as colorless liquid. TLC: 5% MeOH/DCM (Rf: 0.1); ¹H NMR (DMSO-d₆, 500 MHz): δ 7.75 (br s, 1H), 7.29-7.25 (m, 2H), 7.20-7.18 (m, 3H), 3.35-3.32 (m, 1H), 2.91 (dd, J=13.2, 4.9 Hz, 1H), 2.59-2.56 (m, 4H), 1.64 (br s, 2H).

Synthesis of (S)—S-(2-((4-((1-(methylamino)-1-oxo-3-phenylpropan-2-yl)carbamoyl) phenyl)amino)-2-oxoethyl)carbamothloate (7): To a stirred solution of chloroacetic acid (63.3 mg, 0.673 mmol, 1 eq) and ammonium thiocyanate (51.26 mg, 0.673 mmol, 1 eq) was added in ethanol at room temperature. Reaction was stirred for 10 min, then (S)-4-amino-N-(1-(methyl amino)-1-oxo-3-phenylpropan-2-yl) benzamide (200 mg, 0.673 mmol, 1 eq) was added. Then reaction heated to 90° C. and stirred at this temperature for 5h; it was cooled to RT, and stirred at this temperature for 14 h. The reaction was monitored by TLC, after completion of the reaction, ice was added to reaction mixture and filtered to get brown solid. Crude solid was triturated with DCM (5 mL), and filtered to obtain (S)—S-(2-((4-((1-(methyl amino)-1-oxo-3-phenylpropan-2-yl) carbamoyl) phenyl) amino)-2-oxoethyl)-carbamothioate (30 mg, 10.8%) as off white solid. TLC: 10% MeOH/DCM (Rr: 0.3). ¹H NMR (DMSO-d₆, 500 MHz): δ 10.30 (s, 1H), 8.43 (br d, J=8.7 Hz, 1H), 7.99-7.94 (m, 1H), 7.76 (br d, J=8.7 Hz, 3H), 7.60 (br d, J=8.7 Hz, 3H), 7.30-7.27 (m, 2H), 7.25-7.21 (m, 2H), 7.15-7.11 (m, 1H), 4.63-4.57 (m, 1H), 3.72-3.69 (m, 2H), 3.09-3.03 (m, 1H), 2.98-2.92 (m, 1H), 2.59 (d, J=4.6 Hz, 3H); LCMS: 85.54%, m/z=415.1 [M+H]⁺; (Column: EVO-C18 (3.0×50 mm, 2.6 μm); RT: 1.82 min, A: 0.025% Formic acid, B:ACN; T/B %: 0.01/5, 3/90, 5/90, 5.5/5, 6/5, 0.8 mL/min); HPLC: 95.99%; (Column; X-SELECT CSH C-18 (4.6×150 mm, 3.5 μm); RT: 4.97 min; A: 5 mM Ammonium Acetate, B:ACN; T/B %: 0.01/10, 12/90, 16/90. Diluent: ACN: H₂O).

The foregoing protocol was modified accordingly by well-known methods in the art and used to prepare the compounds of the present disclosure provided in Table 8.

### Example 3

### Synthesis of (−)-Enantiomer of S-(1-((4-acetylphenyl) amino)-1-oxobutan-2-yl) carbamothloate (24)

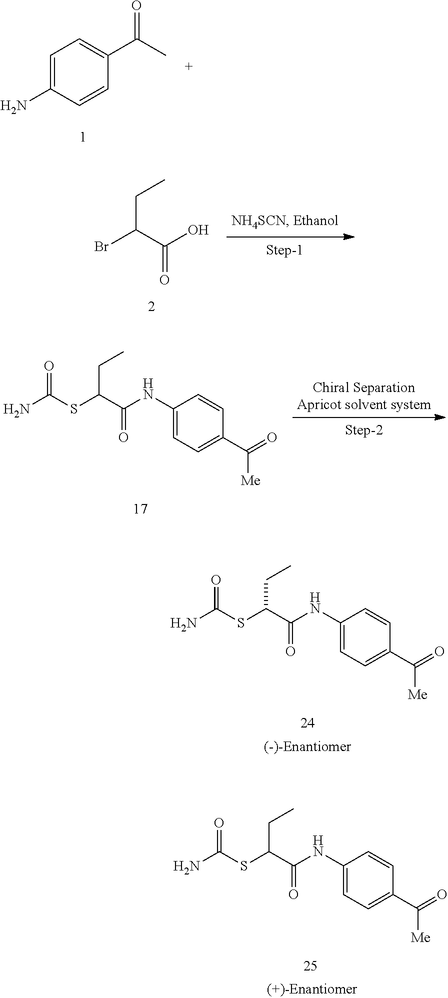

Step 1: Synthesis of racemic S-(1-((4-acetylphenyl) amino)-1-oxobutan-2-yl) carbamothloate (17): To a stirred solution of 2-bromobutanoic acid (2) (0.618 g, 3.69 mmol, 1.0 eq), in ethanol (5 mL), was added NH₄SCN (0.28 g, 3.69 mmol, 1.0 eq), then stirred at RT for 10 min. To this was added 1-(4-aminophenyl) ethan-1-one (1) (0.50 g, 3.69 mmol, 1.0 eq), stirred at 80° C. for 5h, and then at RT for 14 h. The reaction was monitored by TLC, after completion of the reaction, reaction mass was quenched with water (5 mL), and filter to get crude. Residue was triturated with DCM and filtered to obtain racemic S-(1-((4-acetylphenyl) amino)-1-oxobutan-2-yl) carbamothioate (17), (0.215 g, 20.84%), as an off white solid.

¹H NMR (DMSO-d₆, 400 MHz): δ 10.51-10.48 (m, 1H), 7.96-7.88 (m, 2H), 7.74-7.70 (m, 3H), 7.68-7.62 (m, 1H), 4.10-4.02 (m, 1H), 2.56-2.52 (m, 3H), 1.92-1.86 (m, 1H), 1.78-1.68 (m, 1H), 0.98-0.88 (m, 3H).

LCMS: 94.18%, m/z: [M+H]⁺; mass spec calculated for C₁₂H₁₆N₃O₃S, 280.09; mass spec found, 281.11 (Column; X-BRIDGE BEH C-18 (3.0×50 mm, 2.5 μm); RT: 2.154 min; A: 0.5 ml Formic acid in 950 ml water+50 ml ACN, B: 0.5 ml Formic acid in ACN; T/B %: 0.01/2, 0.2/2, 2.2/98, 3/98, 3.2/2, 4/2; Flow rate: 1.2 mL/min (Gradient), Column temperature: 50° C.

Step 2: Synthesis of (−)-Enantiomer of S-(1-((4-acetylphenyl)amino)-1-oxobutan-2-yl)carbamothloate (24): The racemic S-(1-((4-acetylphenyl) amino)-1-oxobutan-2-yl)carbamothioate (17), (0.215 g), was submitted for chiral separation. Chiral separation method: (Column; CHIRAL PAK IA (150×4.6 mm, 3 μm); A: 0.1% DEA in n-Hexane, B: DCM: MeOH (50:50), Program: 80:20, Flow: 0.7 mL/min.

The first fraction eluted was concentrated to obtain pure enantiomer-1 of S-(1-((4-acetylphenyl)amino)-1-oxobutan-2-yl)carbamothioate (24) as an off white solid.

¹H NMR (DMSO-d₆, 400 MHz): δ 10.51-10.48 (m, 1H), 7.96-7.88 (m, 2H), 7.74-7.70 (m, 3H), 7.68-7.62 (m, 1H), 4.10-4.02 (m, 1H), 2.56-2.52 (m, 3H), 1.92-1.86 (m, 1H), 1.78-1.68 (m, 1H), 0.98-0.88 (m, 3H).

LCMS: 94.96%, m/z: [M+H]⁺; mass spec calculated for C₁₂H₁₆N₃O₃S, 280.34; mass spec found, 281.0 (Column; X-BRIDGE BEH C-18 (3.0×50 mm, 2.5 μm); RT: 1.642 min; A: 0.5 ml Formic acid in 950 ml water+50 ml ACN, B: 0.5 ml Formic acid in ACN; T/B %: 0.01/2, 0.2/2, 2.2/98, 3/98, 3.2/2, 4/2; Flow rate: 1.2 mL/min (Gradient), Column temp: 50° C.

HPLC: 95.86%, (Column; X-SELECT CSH C-18 (4.6×150 mm, 3.5 μm); RT: 6.218 min; A: 5 mM Ammonium bicarbonate, B:ACN; T/B %: 0.01/20, 12/90, 16/90; Flow: 1 mL/min.

SOR: −178.12, c=0.1 in MeOH

### Materials and Methods:

All ¹H NMR spectra were recorded on 400 MHz (Bruker) and 500 MHz (Agilent) NMR spectrometers. All chemical shifts are given as δ value with reference to Tetra methyl silane (TMS) as an internal standard. Products were purified by combiflash and preparative HPLC. The chemicals and solvents were purchased from industrial chemical suppliers and they were used without purification prior to use.

### Example 4

### Screening Compounds for Inhibition of BFT In Vitro—NFF-3 Cleavage Assay

A NFF-3 cleavage assay is used to test activity of recombinant BFT, before or after addition of various inhibitors. The NFF-3 cleavage assay was previously described by Goulas et al., PNAS, 2011, 108(5) 1856-1861, which is incorporated by reference herein in its entirety.

Initially, recombinant BFT (rBFT) (0.25, 0.5, 1, 2, 4, 8, or 16 μg/mL) is incubated at 37° C. with the fluorogenic substrate NFF-3 (Cayman Chemical) at a concentration of 2.5 μM, 5 μM, or 10 μM. After 18 hours, fluorescence is measured in a microplate fluorimeter.

Next, rBFT is pre-incubated with one or more test compounds at different concentrations for 30 minutes at 37° C. The rBFT-compound mixture is then added to NFF-3 and incubated for 24 hours at 37° C. Fluorescence is then measured in a microplate fluorimeter.

### Example 5

### Screening Compounds for Inhibition of BFT in Cells—E-Cadherin Release Assay

Compounds are also screened for their ability to inhibit BFT-induced E-cadherin release from HT29 cells.

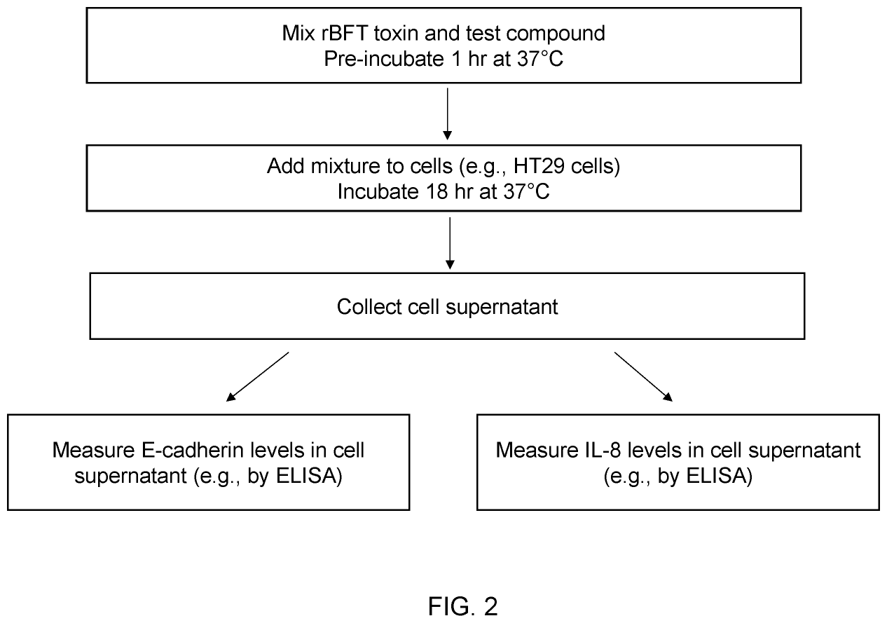

Different concentrations of compound are pre-incubated with rBFT for one hour at 37° C. The rBFT-compound mixture is then added to HT29 cells and incubated at 37° C. for 18 hours. Following incubation, cell supernatants are harvested and E-cadherin is quantified in the supernatants by ELISA (FIG. 2).

### Example 6

### Screening Compounds for Inhibition of BFT In Vivo

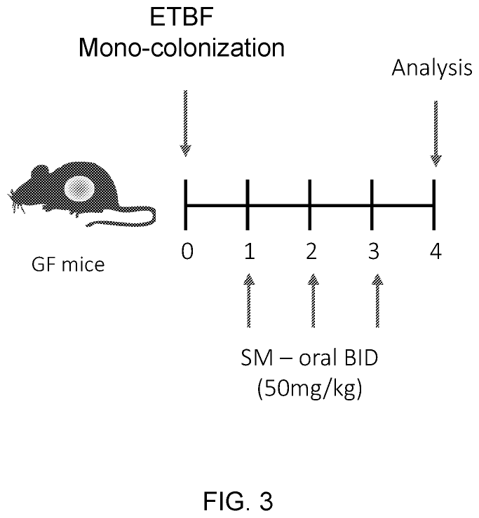

Compounds are also screened in vivo. Germ-free (GF) mice are mono-colonized with ETBF on day 0 (FIG. 3). On days 1, 2, and 3 following colonization, 50 mg/kg of compound are orally administered to the mice two times per day (BID). Markers of injury and inflammation (e.g., cecal weight and fecal lipocalin 2) are analyzed on day 4.

### Example 7

### Administering BFT, ColA, and/or GelE Inhibitors to a Subject

A compound capable of inhibiting BFT, ColA, and/or GelE is formulated as a tablet or capsule for oral administration. The pharmaceutical composition is administered to a subject in a therapeutically effective amount, i.e., an amount sufficient to inhibit BFT, ColA, and/or GelE in the subject.

### Example 8

### Treating a Subject that has IBD

A subject having or suspected of having IBD is tested to determine if they have been colonized by an enterotoxigenic strain of one or more of B. fragilis, E. faecalis, or C. perfringens. If the subject tests positive for one or more of these bacteria or toxins produced thereby, a therapeutically effective amount of a compound capable of inhibiting BFT, GelE, and/or ColA is administered to the subject. The therapeutically effective amount is an amount sufficient to reduce the amount or the pathogenic effects of the one or more enterotoxigenic bacterial strains or toxins produced thereby. Disease progression in the subject is monitored. Subject stool samples may be tested to monitor the presence and/or abundance of the one or more pathogenic bacterial strains or toxins produced thereby, before and after administration of the compound.

### Example 9

### Screening Compounds for Inhibition of Gelatinase E

### Gelatinase Purification

Gelatinase E (Gel E) was purified from bacterial culture supernatant from E. faecalis. E. faecalis was cultured aerobically in Todd Hewitt Broth overnight at 37° C. Nucleic acid is precipitated with 0.9% protamine solution, followed by protein precipitation with ammonium sulfate. Resuspended protein pellet was further subjected to purification using FPLC (phenyl Sepharose column). Fractions with gelatinase activity as determined by casein agar assay were pooled and further concentrated.

### Gelatinase E Activity Assay

Different concentrations of test compound were incubated with purified GelE and FRET-based peptide substrate (390 MMP FRET Substrate 1; AnaspecAS-27077) in assay buffer at room temperature for 30 minutes. The fluorescence signal was determined by a plate reader.

Table 8 (below) provides a summary of inhibition data.

### Example 10

### Screening Compounds for Inhibition of Collagenase H

### Collagenase H Assay as a Surrogate for ColA Inhibition.

Different concentrations of test compounds were incubated with Clostridium histolyticum collagenase H (ColH) and fluorescein-labeled DQ-gelatin conjugate (both are components of EnzCheck Gelatinase/Collagenase Assay Kit, ThermoFisher E12055) at 37° C. for 2 hours. ColH has similar activity to ColA.

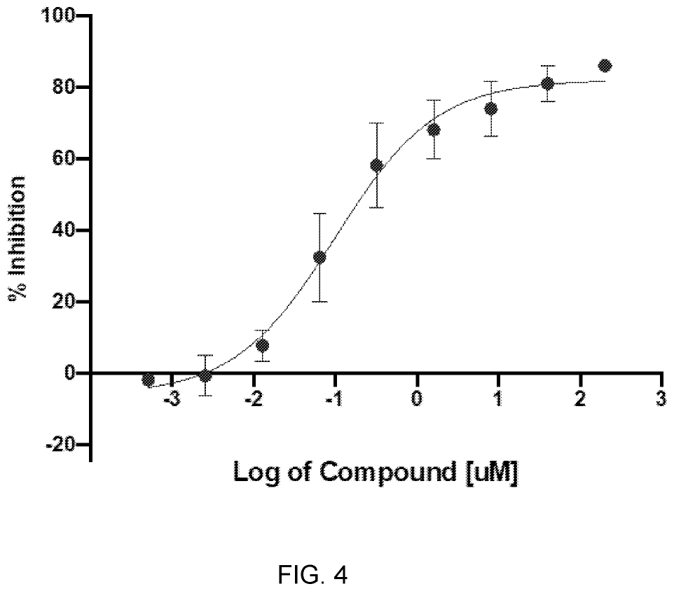

The fluorescence signal was determined by a plate reader and level of inhibition calculated (see FIG. 4).

Table 8 (below) provides a summary of inhibition results.

TABLE 8
Gelatinase E and Collagenase H Inhibition by Compounds of the Present Disclosure

| | Col-H | Gel-E | HPLC | | |
|---|---|---|---|---|---|
| | IC50 | IC50 | Retention | LC-MS | |
| Cmpd. #* | (μM) | (μM) | Time (Min)^{a} | (M + H) | Structure |
| 1 | 0.09958 | 33.72 | 5.45 | 253.00 | 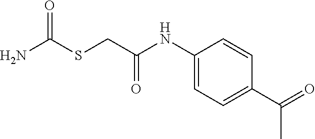 [SMILES: CC(=O)c1ccc(NC(=O)CSC(N)=O)cc1] |
| 2 | 1.94 | 155.1 | 6.33 | 269.00 | 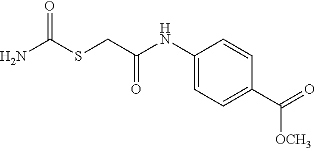 [SMILES: COC(=O)c1ccc(NC(=O)CSC(N)=O)cc1] |
| 3 | 1.11 | 60.1 | 5.59 | 241.10 | 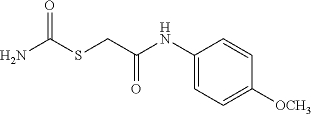 [SMILES: COc1ccc(NC(=O)CSC(N)=O)cc1] |
| 4 | 6.42 | 1.53 | 6.52 | 252.10 | 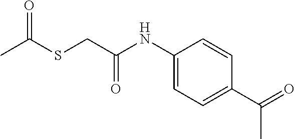 [SMILES: CC(=O)SCC(=O)Nc1ccc(C(C)=O)cc1] |
| 5 | 24.63 | 28.89 | 7.11 | 268.00 | 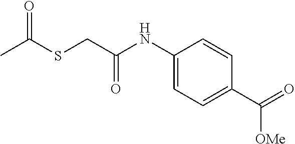 [SMILES: COC(=O)c1ccc(NC(=O)CSC(C)=O)cc1] |
| 6 | 25.86 | 4.16 | 7.50 | 240.10 | 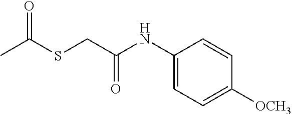 [SMILES: COc1ccc(NC(=O)CSC(C)=O)cc1] |
| 7 | >16 | >100 | 4.98 | 415.10 | 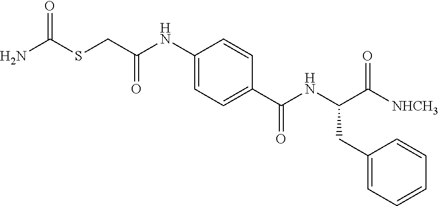 [SMILES: CNC(=O)[C@H](Cc1ccccc1)NC(=O)c1ccc(NC(=O)CSC(N)=O)cc1] |
| 8 | >10 | >100 | 7.20 | 339.00 | 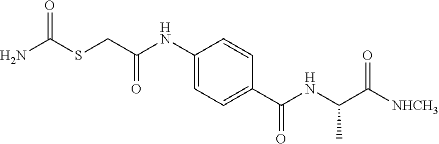 [SMILES: CNC(=O)[C@H](C)NC(=O)c1ccc(NC(=O)CSC(N)=O)cc1] |
| 9 | 0.0838 | >160 | 7.83 | 321.00 | 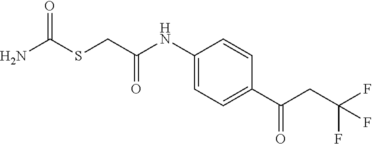 [SMILES: NC(=O)SCC(=O)Nc1ccc(C(=O)CC(F)(F)F)cc1] |
| 10 | >200 | >200 | 3.01 | 298.00 | 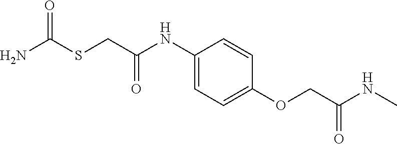 [SMILES: CNC(=O)COc1ccc(NC(=O)CSC(N)=O)cc1] |
| 11 | >50 | 45.37 | 3.60 | 312.00 | 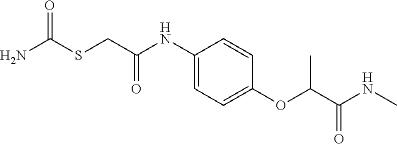 [SMILES: CNC(=O)C(C)Oc1ccc(NC(=O)CSC(N)=O)cc1] |
| 12 | .2 | >100 | 3.88 | 243.00 | 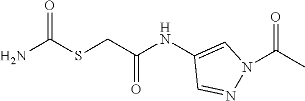 [SMILES: CC(=O)n1cc(NC(=O)CSC(N)=O)cn1] |
| 13 | >200 | NT | 5.94 | 387.90 | 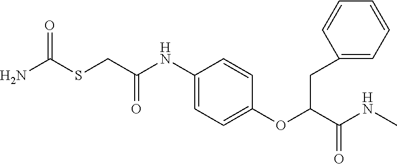 [SMILES: CNC(=O)C(Cc1ccccc1)Oc1ccc(NC(=O)CSC(N)=O)cc1] |
| 14 | 0.554 | NT | 6.75 | 300.90 | 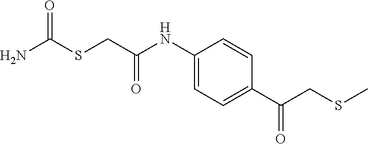 [SMILES: CSCC(=O)c1ccc(NC(=O)CSC(N)=O)cc1] |
| 15 | 0.031 | NT | 5.84 | 267.10 | 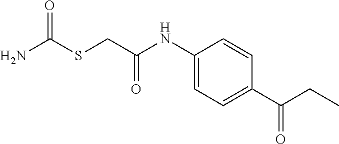 [SMILES: CCC(=O)c1ccc(NC(=O)CSC(N)=O)cc1] |
| 16 | NT | >37.8 | 5.84 | 247.10 | 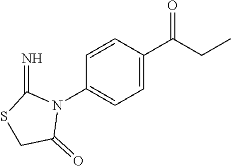 [SMILES: CCC(=O)c1ccc(N2C(=N)SCC2=O)cc1] |
| 17 | 0.00056 | | 5.74 | 281.10 | 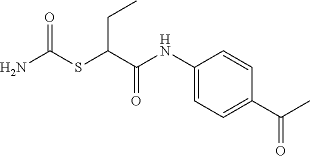 [SMILES: CCC(SC(N)=O)C(=O)Nc1ccc(C(C)=O)cc1] |
| 18 | 0.0272 | | 5.60 | 210.00 | 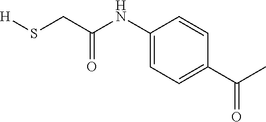 |
| 19 | >0.16 | | 4.66 | 283.00 | 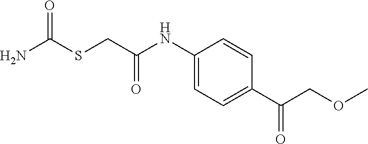 [SMILES: COCC(=O)c1ccc(NC(=O)CSC(N)=O)cc1] |
| 20 | >100 | | 2.45 | 281.90 | 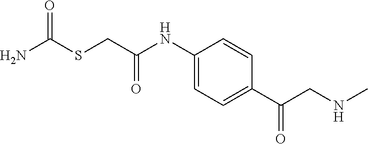 [SMILES: CNCC(=O)c1ccc(NC(=O)CSC(N)=O)cc1] |
| 21 | >0.16 | | 6.36 | 281.10 | 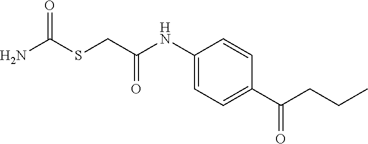 [SMILES: CCCC(=O)c1ccc(NC(=O)CSC(N)=O)cc1] |
| 22 | 50.2 | | 5.44 | 281.10 | 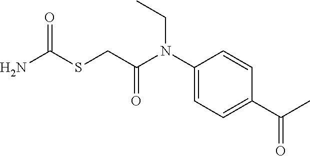 [SMILES: CCN(C(=O)CSC(N)=O)c1ccc(C(C)=O)cc1] |
| 23 | >100 | | 5.85 | 263.10 | 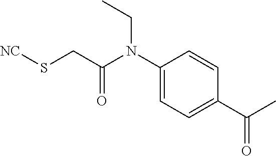 [SMILES: CCN(C(=O)CSC#N)c1ccc(C(C)=O)cc1] |
| 24 | 0.00025 | | 5.74 | 281.10 | (−)-17 Sp.Opt. Rot. = −178.12b |
| 25 | 0.00926 | | 5.74 | 281.10 | (+)-17 Sp.Opt. Rot. = +189.76b |
| 26 | 0.00497 | | 7.45 | 268.75 | 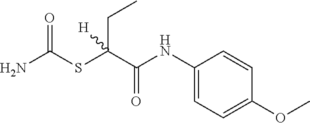 [SMILES: CCC(SC(N)=O)C(=O)Nc1ccc(OC)cc1] |
| 27 | 0.00709 | | 6.25 | 348.70 | 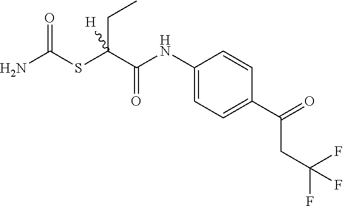 [SMILES: CCC(SC(N)=O)C(=O)Nc1ccc(C(=O)CC(F)(F)F)cc1] |
| 28 | 1.58 | | 6.69 | 335.10 | 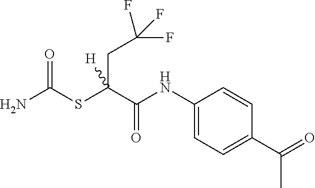 [SMILES: CC(=O)c1ccc(NC(=O)C(CC(F)(F)F)SC(N)=O)cc1] |
| 29 | 1.775 | | 9.08 | 337.20 | 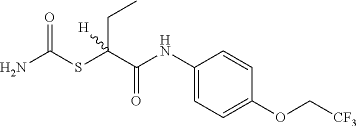 [SMILES: CCC(SC(N)=O)C(=O)Nc1ccc(OCC(F)(F)F)cc1] |
| 30 | 0.178 | | 8.69 | 283.20 | 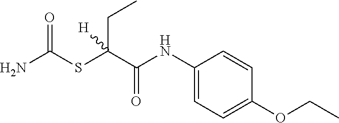 [SMILES: CCOc1ccc(NC(=O)C(CC)SC(N)=O)cc1] |
| 31 | 0.040 | | 8.03 | 295.20 | 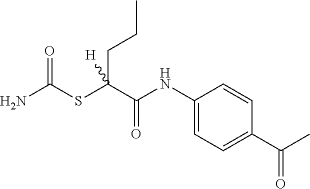 [SMILES: CCCC(SC(N)=O)C(=O)Nc1ccc(C(C)=O)cc1] |
| 32 | 22.1 | | 7.56 | 252.60 | 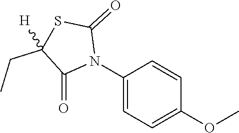 [SMILES: CCC1SC(=O)N(c2ccc(OC)cc2)C1=O] |
| 33 | >100 | | 7.36 | 239.00 | 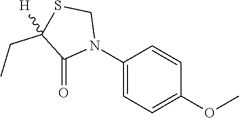 [SMILES: CCC1SCN(c2ccc(OC)cc2)C1=O] |
| 34 | 0.0345 | | 7.19 | 311.10 | 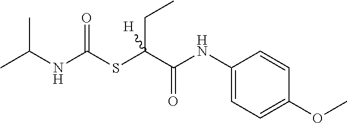 [SMILES: CCC(SC(=O)NC(C)C)C(=O)Nc1ccc(OC)cc1] |
| 35 | 0.392 | | 10.22 | 325.20 | 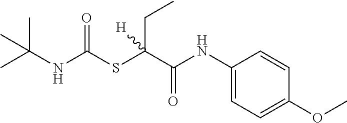 [SMILES: CCC(SC(=O)NC(C)(C)C)C(=O)Nc1ccc(OC)cc1] |
| 36 | >100 | | 7.87 | 297.20 | 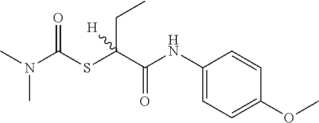 |
| 37 | 0.0235 | | 8.62 | 415.05 | 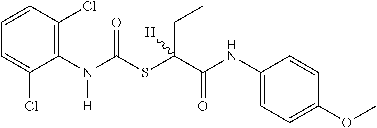 [SMILES: CCC(SC(=O)Nc1c(Cl)cccc1Cl)C(=O)Nc1ccc(OC)cc1] |
| 38 | 0.0135 | | 10.37 | 362.90 | 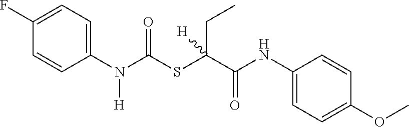 [SMILES: CCC(SC(=O)Nc1ccc(F)cc1)C(=O)Nc1ccc(OC)cc1] |
| 39 | NT | | 4.54 | 381.20 | 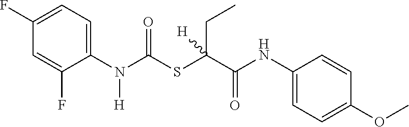 [SMILES: CCC(SC(=O)Nc1ccc(F)cc1F)C(=O)Nc1ccc(OC)cc1] |
| 40 | NT | | 8.33 | 380.90 | 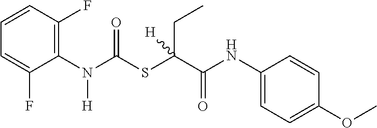 [SMILES: CCC(SC(=O)Nc1c(F)cccc1F)C(=O)Nc1ccc(OC)cc1] |

**All ¹H NMR spectra were recorded on 400 MHz (Bruker) and 500 MHz (Agilent) NMR spectrometers. All chemical shifts are given as δ value with reference to tetramethylsilane (TMS) as an internal standard. Products were purified by flash chromatography on 100-200 mesh silica gel and final compound purified through preparative HPLC. The chemicals and solvents were purchased from industrial chemical suppliers and they were used without purification prior to use.
^{a}Column; X-SELECT CSH C-18 (150 × 4.6 mm, 3.5 μm); 5 mM ammonium acetate + acetonitrile; 1.0 mL/min, Diluent: ACN: H₂O).
^{b}C = 0.1% in MeOH (JASCO P-2000 polarimeter).

### Example 11

Inhibition of Type IV Collagenase from C. histolyticum and the collagenase activity of cultured supernatants from 3 ATCC strains of C. perfringens.

Collagenase activity from culture supernatants of C. perfringens strains (ColA/ColH) was measured using fluorogenic DQ™ collagen (thermo D12054) in the presence of the collagenase inhibitor compound 24. Single colonies of C. perfringens strains were inoculated into 10 ml RCM media (ATCC Medium 2107) grown for ˜20 hours at 37° C. in an anaerobic chamber (Coy Laboratory Products) containing 20% CO₂, 10% H₂, and 70% N₂. Three ml of culture supernatant, harvested by centrifugation at 4,000 RPM for 10 minutes, was buffer-exchanged using Amicon® Ultra-2 30 Kd into 1× EnzChek® Collagenase Assay Kit (thermo D12060) 1× reaction buffer (0.05 M Tris-HCl, 0.15 M NaCl, 5 mM CaCl₂, 0.2 mM sodium azide, pH 7.6). The >30 KD protein fraction was recovered in 2 mL of 1× reaction buffer, further diluted 1:5 into the collagenase activity medium containing 50 μg/mL DQ collagen (thermo D-12060) and compound 24 (conc. range 0.01-100 nM). Digestion products from DQ collagen were monitored on a Biotek Synergy H1 instrument with 495 nm excitation (515 nm emission) for 180 minutes after reaction initiation. ColH (1 U/ml), purified Type IV Collagenase from Clostridium histolyticum is used as a positive control (thermo D12060).

1.-100. (canceled)

101. A compound of Formula VB: 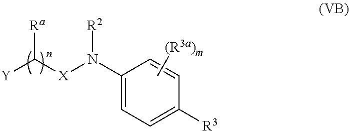
or a stereoisomer or a pharmaceutically acceptable salt thereof, wherein:
X is —C(O)— or —S(O)₂—;
Y is —SC(O)R¹ or —SH;
R^{a} is H, alkyl, haloalkyl, aryl, alkylene-cycloalkyl, alkylene-heterocycloalkyl, heteroaryl, alkylene-aryl, or alkylene-heteroaryl;
R¹ is alkyl, haloalkyl, or —NR⁴R⁵;
R² is H, alkyl, or cycloalkyl;
R³ is —OH, alkoxy, —O-alkylene-NR⁷R⁸, —C(O)R, or —C(O)N(H)-alkylene-C(O)NR⁷R⁸, wherein the alkylene is optionally substituted;
R^{3a} is H, alkyl, halogen, alkoxy, haloalkyl or haloalkoxy;
R⁴, R, R⁷, and R⁸ are each independently H, alkyl, cycloalkyl, —CH₂cycloalkyl, aryl, or —CH₂aryl;
R⁶ is alkyl, haloalkyl, alkoxy, or —N(H)alkyl;
m is 0, 1, 2, or 3; and
n is 1 or 2,
wherein when n is 1, R is not H.

102. The compound of claim 101, wherein X is —C(O)—.

103. The compound of claim 101, wherein the compound of Formula (VB) has a structure according to: 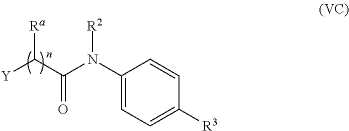
or a stereoisomer or a pharmaceutically acceptable salt thereof.

104. The compound of claim 101, wherein Y is —SC(O)R¹, wherein R¹ is C₁₋₅alkyl or —NR⁴R⁵.

105. The compound of claim 101, wherein R⁴ and R⁵ are each independently H, alkyl, or optionally substituted phenyl.

106. The compound of claim 101, wherein R² is H.

107. The compound of claim 101, wherein R³ is alkoxy, —O-alkylene-NR⁷R⁸, —C(O)R⁶, or —C(O)N(H)-alkylene-C(O)NR⁷R⁸.

108. The compound of claim 107, wherein:
(i) the alkylene is a CI-3alkylene optionally substituted with oxo, alkyl, alkylene-cycloalkyl, alkylene-heterocycloalkyl, alkylene-aryl, or alkylene-heteroaryl; or
(ii) the —O-alkylene-NR⁷R⁸ is 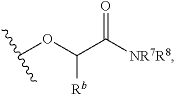
wherein R^{b} is H, alkyl, alkylene-cycloalkyl, alkylene-heterocycloalkyl, alkylene-aryl, or alkylene-heteroaryl, optionally wherein R^{b} is H, —C₁₋₅alkyl, or —CH₂aryl; and/or
(iii) the —C(O)N(H)-alkylene-C(O)NR⁷R⁸ is 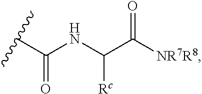
wherein R^{c} is H, alkyl, alkylene-cycloalkyl, alkylene-heterocycloalkyl, alkylene-aryl, or alkylene-heteroaryl, optionally wherein R^{c} is —C₁₋₅alkyl, or —CH₂aryl; and/or
(iv) the alkoxy is —OMe.

109. The compound of claim 101, wherein R⁶ is alkyl, haloalkyl, or alkoxy.

110. The compound of claim 109, wherein R⁶ is Me, Et, CH₂CF₃, —OMe, —CH₂O(C₁₋₅alkyl), —CH₂S(C₁₋₅alkyl), or —CH₂N(H)(C₁₋₅alkyl).

111. The compound of claim 101, wherein R⁷ and R⁸ are each independently H, Me, or —CH₂Ph.

112. The compound of claim 101, wherein R^{a} is H, alkyl, haloalkyl, or alkylene-cycloalkyl.

113. The compound of claim 112, wherein R^{a} is ethyl, n-propyl, isopropyl, n-butyl, isobutyl, sec-butyl, isoamyl, or neopentyl.

114. The compound of claim 101, wherein m is 0 or 1.

115. The compound of claim 101, wherein n is 1.

116. The compound of claim 101, wherein R^{3a} is H, F, Me, i-Pr, CF₃, CF₂H, —CH₂F, or —OMe.

117. The compound of claim 101, wherein the compound is: 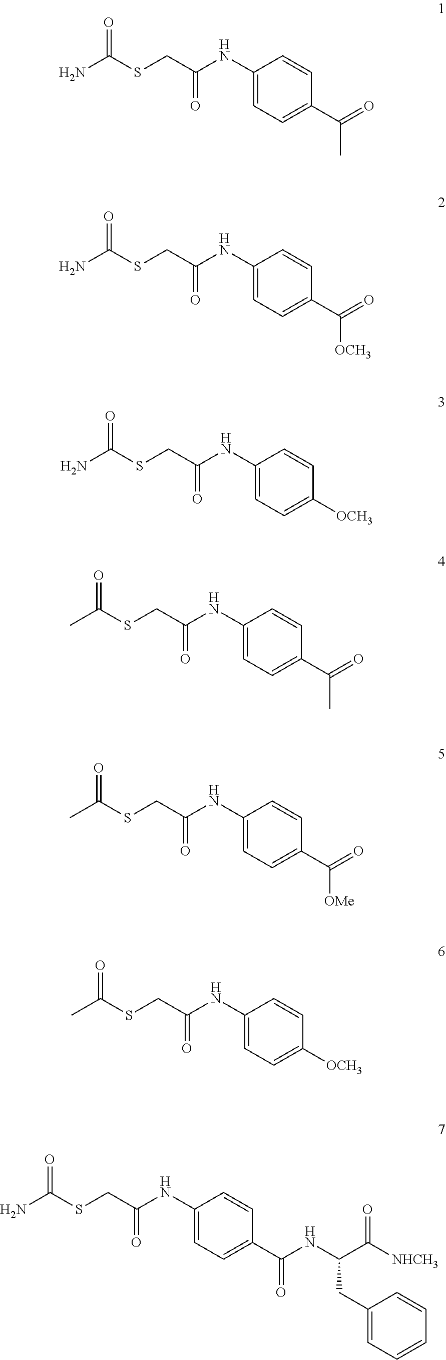 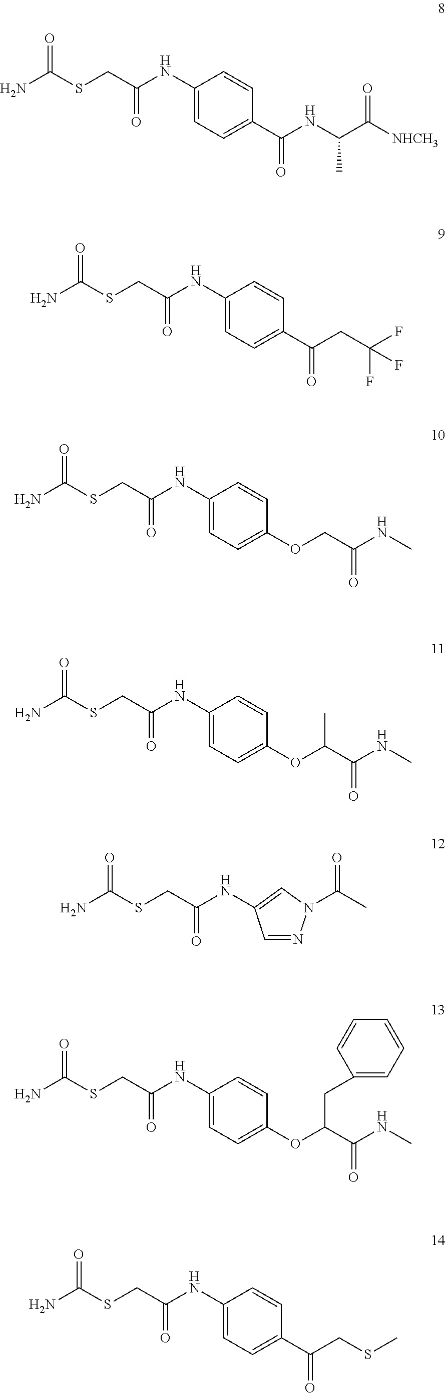 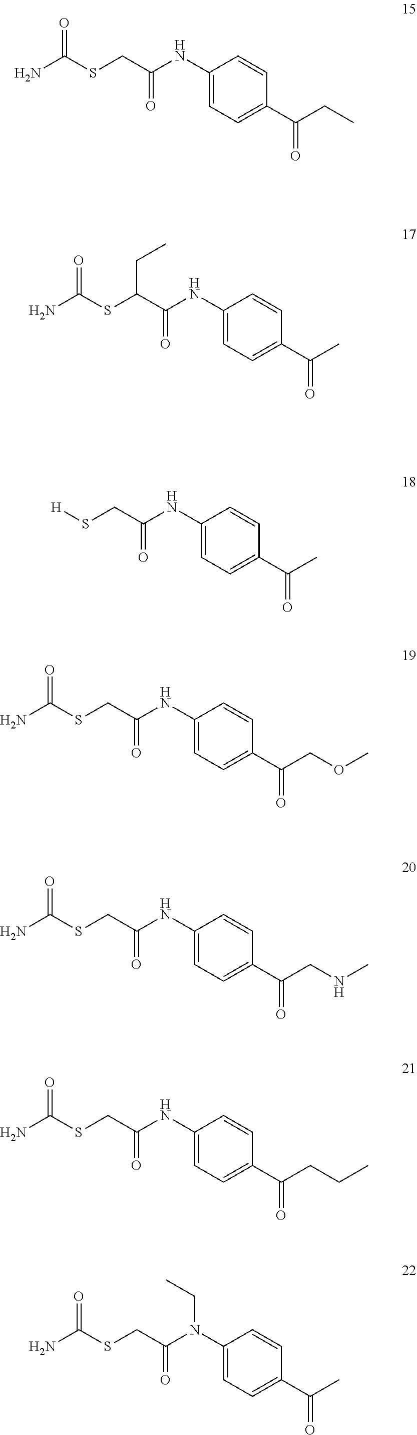 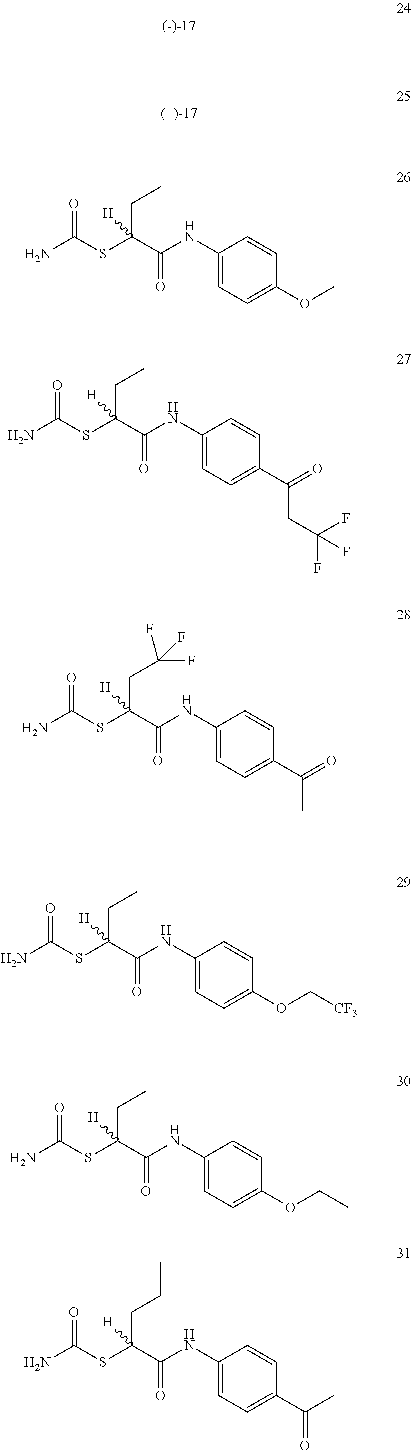 [SMILES: CCC(SC(N)=O)C(=O)Nc1ccc(OC)cc1 ; CCC(SC(N)=O)C(=O)Nc1ccc(C(=O)CC(F)(F)F)cc1 ; CC(=O)c1ccc(NC(=O)C(CC(F)(F)F)SC(N)=O)cc1 ; CCC(SC(N)=O)C(=O)Nc1ccc(OCC(F)(F)F)cc1 ; CCOc1ccc(NC(=O)C(CC)SC(N)=O)cc1 ; CCCC(SC(N)=O)C(=O)Nc1ccc(C(C)=O)cc1] 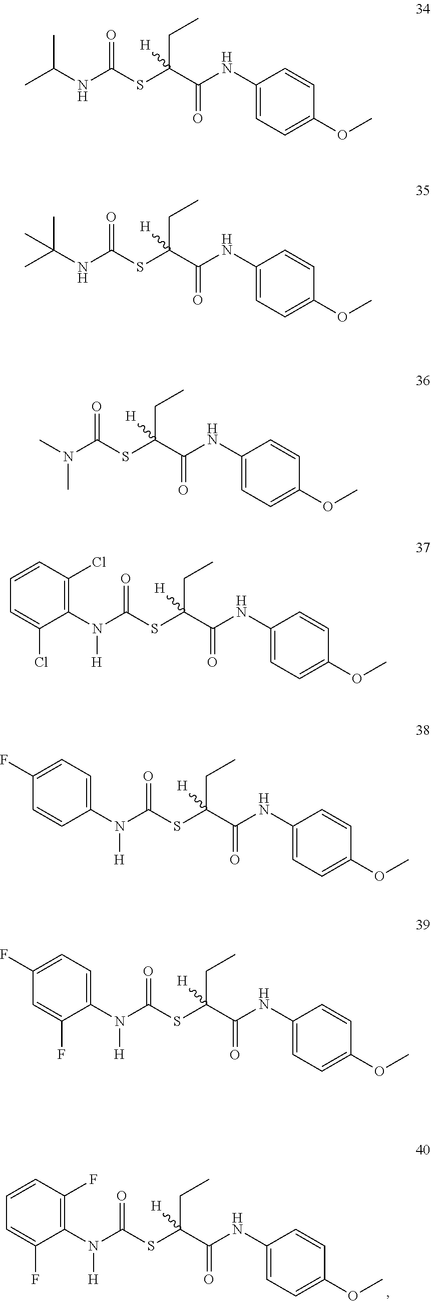
or a stereoisomer or a pharmaceutically acceptable salt thereof.

118. A pharmaceutical composition comprising a compound of claim 101 and a pharmaceutically acceptable carrier or excipient.

119. A method of treating inflammatory bowel disease in a subject in need thereof comprising, administering to the subject a therapeutically effective amount of the compound of claim 101.

120. A method of treating gastrointestinal (GI) cancer in a subject in need thereof, comprising, administering to the subject a therapeutically effective amount of the compound of claim 101.

121. A method of treating a systemic bacterial infection in a subject in need thereof comprising, administering to the subject a therapeutically effective amount of the compound of claim 101.

122. The method of claim 119, wherein the subject is colonized by one or more pathogenic bacterial strain, wherein the pathogenic bacterial strain is a strain of B. fragilis expressing the BFT toxin, a strain of E. faecalis expressing the gelatinase GelE, or a strain of C. perfringens expressing the collagenase ColA.


SEQUENCE LISTING (10 sequences)

SEQ ID NO: 1 - DNA, 1275 bp, Bacteroides fragilis
  source 1..1275: mol_type = genomic DNA
gcgcgattactagcgaatccagcttcacgaagtcgggttgcagacttcgatccgaactgagagaggattttgggattagcatacggtcacccgctagctgccttctgtaccccccattgtaacacgtgtgtagccccggacgtaagggccgtgctgatttgacgtcatccccaccttcctcacatcttacgacggcagtctctccagagtcctcagcatgacctgttagtaactgaagataagggttgcgctcgttatggcacttaagccgacacctcacggcacgagctgacgacaaccatgcagcaccttcacagcggtgattgctcactgacatgtttccacatcattccactgcaatttaagcccgggtaaggttcctcgcgtatcatcgaattaaaccacatgttcctccgcttgtgcgggcccccgtcaattcctttgagtttcaccgttgccggcgtactccccaggtggaatacttaatgctttcgcttggccgcttactgtatatcgcaaacagcgagtattcatcgtttactgtgtggactaccagggtatctaatcctgtttgatacccacactttcgagcatcagtgtcagttgcagtccagtgagctgccttcgcaatcggagttcttcgtgatatctaagcatttcaccgctacaccacgaattccgcccacctctactgtactcaagactgacagtatcaactgcaattttacggttgagccgcaractttcacaactgacttaccagtccacctacgctccctttaaacccaataaatccggataacgctcggatcctccgtattaccgcggctgctggcacggagttagccgatccttattcatataatacatacaaaacagtatacatactgcactttattcttatataaaagaagtttacgacccatagagccttcatccttcacgctacttggctggttcaggctagcgcccattgaccaatattcctcactgctgcctcccgtaggagtttggaccgtgtctcagttccaatgtgggggaccttcctctcagaacccctatccatcgaaggcttggtgagccgttacctcaccaacaacctaatggaacgcatccccatcctttaccggaatcctttaataatgaaaccatgcggaatcattatgctatcgggtattaatctttctttcgaaaggctatccccgagtaaagggcaggttggatacgtgttactcacccgtgcgccggtcgccggcaaagaaagcaagctttctt

SEQ ID NO: 2 - protein, 405 aa, Bacteroides fragilis
MFILNFNKMKNVKLLLMLGTAALLAACSNEADSLTTSIDAPVTASIDLQSVSYTDLATQLNDVSDFGKMIILKDNGFNRQVHVSMDKRTKIQLDNENVRLFNGRDKDSTSFILGDEFAVLRFYRNGESISYIAYKEAQMMNEIAEFYAAPFKKTRAINEKEAFECIYDSRTRSAGKDIVSVKINIDKAKKILNLPECDYINDYIKTPQVPHGITESQTRAVPSEPKTVYVICLRENGSTIYPNEVSAQMQDAANSVYAVHGLKRYVNFHFVLYTTEYSCPSGDAKEGLEGFTASLKSNPKAEGYDDQIYFLIRWGTWDNKILGMSWFNSYNVNTASDFEASGMSTTQLMYPGVMAHELGHILGAEHTDNSKDLMYATFTGYLSHLSEKNMDIIAKNLGWEAADGD

SEQ ID NO: 3 - protein, 397 aa, Bacteroides fragilis
MKNVKLLLMLGTAALLAACSNEADSLTTSIDTPVTASIDLQSVSYTDLATQLNDVSDFGKMIILKDNGFNRQVHVSMDKRTKIQLDNENVRLFNGRDKDSTSFILGDEFAVLRFYRNGESISYIAYKEAQMMNEIAEFYAAPFKKTRAINEKEAFECIYDSRTRSAGKDLVSVKINIDKAKKILNLPECDYINDYIKTPQVPHGITESQTRAVPSEPKTVYVICLRESGSTVYPNEVSAQMQDAANSVYAVHGLKRFVNLHFVLYTTEYSCPSGNADEGLDGFTASLKANPKAEGYDDQIYFLIRWGTWDNNILGISWLDSYNVNTASDFKASGMSTTQLMYPGVMAHELGHILGARHADDPKDLMYSKYTGYLFHLSEENMYRIAKNLGWEIADGD

SEQ ID NO: 4 - protein, 397 aa, Bacteroides fragilis
MKNVKLLLMLGTAALLAACSNEADSLTTSIDAPVTASIDLQSVSYTDLATQLNDVSDFGKMIILKDNGFNRQVHVSMDKRTKIQLDNENVRLFNGRDKDSTNFILGDEFAVLRFYRNGESISYIAYKEAQMMNEIAEFYAAPFKKTRAINEKEAFECIYDSRTRSAGKYPVSVKINVDKAKKILNLPECDYINDYIKTPQVPHGITESQTRAVPSEPKTVYVICLRENGSTVYPNEVSAQMQDAANSVYAVHGLKRYVNLHFVLYTTEYACPSGNADEGLDGFTASLKANPKAEGYDDQIYFLIRWGTWDNNILGISWLNSYNVNTASDFKASGMSTTQLMYPGVMAHELGHILGANHADDPKDLMYSKYTGYLFHLSEKNMDIIAKNLGWEIADGD

SEQ ID NO: 5 - DNA, 1478 bp, Enterococcus faecalis
  variation 468: note = n is any nucleotide
  source 1..1478: mol_type = genomic DNA
gacgaacgctggcggcgtgcctaatacatgcaagtcgaacgcttctttcctcccgagtgcttgcactcaattggaaagaggagtggcggacgggtgagtaacacgtgggtaacctacccatcagagggggataacacttggaaacaggtgctaataccgcataacagtttatgccgcatggcataagagtgaaaggcgctttcgggtgtcgctgatggatggacccgcggtgcattagctagttggtgaggtaacggctcaccaaggccacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggacgttagtaactgaacgtcncctgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgctagaccgcgaggtcatgcaaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacctttttggagccagccgcctaaggtgggatagatgattg

SEQ ID NO: 6 - protein, 510 aa, Enterococcus faecalis
MMKGNKILYILGTGIFVGSSCLFSSLFVAAEEQVYSESEVSTVLSKLEKEAISEAAAEQYTVVDRKEDAWGMKHLKLEKQTEGVTVDSDNVIIHLDRNGAVTSVTGNPVDQVVKIQSVDAIGEEGVKKIIASDNPETKDLVFLAIDKRVNNEGQLFYKVRVTSSPTGDPVSLVYKVNATDGTIMEKQDLTEHVGSEVTLKNSFQVAFNVPVEKSNTGIALHGTDNTGVYHAVVDGKNNYSIIQAPSLVALNQNAVDAYTHGKFVKTYYEDHFQRHSIDDRGMPILSVVDEQHPDAYDNAFWDGKAMRYGETSTPTGKTYASSLDVVGHEMTHGVTEHTAGLEYLGQSGALNESYSDLMGYIISGASNPEIGADTQSVDRKTGIRNLQTPSKHGQPETMAQYDDRARYKGTPYYDQGGVHYNSGIINRIGYTIIQNLGIEKAQTIFYSSLVNYLTPKAQFSDARDAMLAAAKVQYGDEAASVVSAAFNSAGIGAKEDIQVNQPSESVLVNE

SEQ ID NO: 7 - DNA, 1513 bp, Clostridium perfringens
  source 1..1513: mol_type = genomic DNA
taaattgagagtttgatcctggctcaggatgaacgctggcggcgtgcttaacacatgcaagtcgagcgatgaagtttccttcgggaaacggattagcggcggacgggtgagtaacacgtgggtaacctgcctcatagagtggaatagccttccgaaaggaagattaataccgcataacgttgaaagatggcatcatcattcaaccaaaggagcaatccgctatgagatggacccgcggcgcattagctagttggtggggtaacggcctaccaaggcgacgatgcgtagccgacctgagagggtgatcggccacattgggactgagacacggcccagactcctacgggaggcagcagtggggaatattgcacaatgggggaaaccctgatgcagcaacgccgcgtgagtgatgaaggttttcggatcgtaaagctctgtctttggggaagataatgacggtacccaaggaggaagccacggctaactacgtgccagcagccgcggtaatacgtaggtggcgagcgttatccggatttactgggcgtaaagggagcgtaggcggatgattaagtgggatgtgaaatacccgggctcaacttgggtgctgcattccaaactggttatctagagtgcaggagaggagagtggaattcctagtgtagcggtgaaatgcgtagagattaggaagaacaccagtggcgaaggcgactctctggactgtaactgacgctgaggctcgaaagcgtggggagcaaacaggattagataccctggtagtccacgccgtaaacgatgaatactaggtgtgggggtttcaacacctccgtgccgccgctaacgcattaagtattccgcctggggagtacggtcgcaagattaaaactcaaaggaattgacggggacccgcacaagtagcggagcatgtggtttaattcgaagcaacgcgaagaaccttacctacacttgacatcccttgcattactcttaatcgaggaaatcccttcggggacaaggtgacaggtggtgcatggttgtcgtcagctcgtgtcgtgagatgttgggttaagtcccgcaacgagcgcaacccttgtcgttagttactaccattaagttgaggactctagcgagactgcctgggttaaccaggaggaaggtggggatgacgtcaaatcatcatgccccttatgtgtagggctacacacgtgctacaatggctggtacagagagatgcaataccgcgaggtggagccaaacttaaaaaccagtctcagttcggattgtaggctgaaactcgcctacatgaagctggagttactagtaatcgcgaatcagaatgtcgcggtgaatacgttcccgggtcttgtacacaccgcccgtcacaccatgagagttggcaatacccgaagtccgtgagctaaccgcaaggaggcagcggccgaaggtagggtcagcgattggggtgaagtcgtaacaaggtagccgtaggagaacctgcggctggatcacctccttt

SEQ ID NO: 8 - protein, 1104 aa, Clostridium perfringens
MKKNLKRGELTKLKLVERWSATFTLAAFILFNSSFKVLAADKKVENSNNGQITREINADQISKTELNNEVATDNNRPLGPSIAPSRARNNKIYTFDELNRMNYSDLVELIKTISYENVPDLFNFNDGSYTFFSNRDRVQAIIYGLEDSGRTYTADDDKGIPTLVEFLRAGYYLGFYNKQLSYLNTPQLKNECLPAMKAIQYNSNFRLGTKAQDGVVEALGRLIGNASADPEVINNCIYVLSDFKDNIDKYGSNYSKGNAVFNLMKGIDYYTNSVIYNTKGYDAKNTEFYNRIDPYMERLESLCTIGDKLNNDNAWLVNNALYYTGRMGKFREDPSISQRALERAMKEYPYLSYQYIEAANDLDLNFGGKNSSGNDIDFNKIKADAREKYLPKTYTFDDGKFVVKAGDKVTEEKIKRLYWASKEVKAQFMRVVQNDKALEEGNPDDILTVVIYNSPEEYKLNRIINGFSTDNGGIYIENIGTFFTYERTPEESIYTLEELFRHEFTHYLQGRYVVPGMWGQGEFYQEGVLTWYEEGTAEFFAGSTRTDGIKPRKSVTQGLAYDRNNRMSLYGVLHAKYGSWDFYNYGFALSNYMYNNNMGMFNKMTNYIKNNDVSGYKDYIASMSSDYGLNDKYQDYMDSLLNNIDNLDVPLVSDEYVNGHEAKDINEITNDIKEVSNIKDLSSNVEKSQFFTTYDMRGTYVGGRSQGEENDWKDMNSKLNDILKELSKKSWNGYKTVTAYFVNHKVDGNGNYVYDVVFHGMNTDTNTDVHVNKEPKAVIKSDSSVIVEEEINFDGTESKDEDGEIKAYEWDFGDGEKSNEAKATHKYNKTGEYEVKLTVTDNNGGINTESKKIKVVEDKPVEVINESEPNNDFEKANQIAKSNMLVKGTLSEEDYSDKYYFDVAKKGNVKITLNNLNSVGITWTLYKEGDLNNYVLYATGNDGTVLKGEKTLEPGRYYLSVYTYDNQSGTYTVNVKGNLKNEVKETAKDAIKEVENNNDFDKAMKVDSNSKIVGTLSNDDLKDIYSIDIQNPSDLNIVVENLDNIKMNWLLYSADDLSNYVDYANADGNKLSNTCKLNPGKYYLCVYQFENSGTGNYIVNLQNK

SEQ ID NO: 9 - protein, 1021 aa, Clostridium histolyticum
MKRKCLSKRLMLAITMATIFTVNSTLPIYAAVDKNNATAAVQNESKRYTVSYLKTLNYYDLVDLLVKTEIENLPDLFQYSSDAKEFYGNKTRMSFIMDEIGRRAPQYTEIDHKGIPTLVEVVRAGFYLGFHNKELNEINKRSFKERVIPSILAIQKNPNFKLGTEVQDKIVSATGLLAGNETAPPEVVNNFTPILQDCIKNIDRYALDDLKSKALFNVLAAPTYDITEYLRATKEKPENTPWYGKIDGFINELKKLALYGKINDNNSWIIDNGIYHIAPLGKLHSNNKIGIETLTEVMKVYPYLSMQHLQSADQIKRHYDSKDAEGNKIPLDKFKKEGKEKYCPKTYTFDDGKVIIKAGARVEEEKVKRLYWASKEVNSQFFRVYGIDKPLEEGNPDDILTMVIYNSPEEYKLNSVLYGYDTNNGGMYIEPEGTFFTYEREAQESTYTLEELFRHEYTHYLQGRYAVPGQWGRTKLYDNDRLTWYEEGGAELFAGSTRTSGILPRKSIVSNIHNTTRNNRYKLSDTVHSKYGASFEFYNYACMFMDYMYNKDMGILNKLNDLAKNNDVDGYDNYIRDLSSNYALNDKYQDHMQERIDNYENLTVPFVADDYLVRHAYKNPNEIYSEISEVAKLKDAKSEVKKSQYFSTFTLRGSYTGGASKGKLEDQKAMNKFIDDSLKKLDTYSWSGYKTLTAYFTNYKVDSSNRVTYDVVFHGYLPNEGDSKNSLPYGKINGTYKGTEKEKIKFSSEGSFDPDGKIVSYEWDFGDGNKSNEENPEHSYDKVGTYTVKLKVTDDKGESSVSTTTAEIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVGR

SEQ ID NO: 10 - protein, 8 aa, synthetic construct
  REGION 1..8: note = FRET-based enzyme substrate
  MOD_RES 1: note = X is 7-methoxy-coumarin-4-yl-acetic acid-2,4-dinitrophenyl-lysine
  MOD_RES 6: note = X is N beta-2,4-dinitrophenyl-L-di-aminopropionic acid
  MOD_RES 8: note = AMIDATION
XPLGLXAR